(19) United States
(12) Patent Application Publication
(10) Pub. No.: US 2026/0315615 A1
(43) Pub. Date: Oct. 15, 2026

(54) Antibody Molecules and Conjugates

(71) Applicant: AstraZeneca Ireland Limited, Dublin (IE)

(72) Inventors: Mattia Cocco, Cambridge (GB); Sujiet Puthenveetil, Wilmington, DE (US); Ryan Fleming, Gaithersburg, MD (US); Devesh Aggarwal, Wilmington, DE (US); Jonathan David Beadle, Cambridge (GB); Albano Galan Coca, Cambridge (GB); Alex Cazes, Wilmington, DE (US)

(21) Appl. No.: 19/532,042

(22) Filed: Feb. 6, 2026

Related U.S. Application Data
(60) Provisional application No. 63/755,617, filed on Feb. 7, 2025.

(51) Int. Cl. A61K 47/68; A61K 47/54; A61P 35/00

(52) U.S. Cl. CPC A61K 47/6849; A61K 47/549; A61K 47/68031; A61K 47/6889; A61P 35/00

(57) ABSTRACT

This specification relates to antibodies that bind B7H3, antibody-drug conjugates comprising an anti-human B7H3 antibody or antigen binding fragment thereof linked to a therapeutic drug, pharmaceutical compositions containing them, and their use in therapy for the treatment of cancer.

The present specification claims benefit of priority to U.S. Patent Application No. 63/755,617, filed 7 Feb. 2025, the entire contents of which are hereby incorporated in their entirety for all purposes.

### REFERENCE TO THE SEQUENCE LISTING

The content of the electronically submitted sequence listing (Name: 300040-PCT01-NP SEQ Listing For filing.xml; Size: 20,141 bytes; Date of Creation: 6 Feb. 2026) filed with this application, is hereby incorporated herein by reference in its entirety.

### FIELD OF THE DISCLOSURE

The present disclosure relates to antibodies that bind B7H3, anti-B7H3 antibody-drug conjugates and uses thereof.

### BACKGROUND

B7 Homolog 3 (B7H3), also known as CD276 (Cluster of Differentiation 276) is a member of the B7 family. It functions as a costimulatory molecule and immune checkpoint molecule and is expressed at low levels various cells including immune cells. As such, B7H3 is thought to play a role in modulating immune responses (Zhao et al., J Hematol Oncol. 2022, 15:153). However, B7H3 is overexpressed in several malignancies such as papillary thyroid carcinoma, glioma, squamous non-small cell lung cancer, endometrial cancer, prostate cancer and head and neck squamous cell carcinoma. As such, B7H3 is thought to play various roles in cancer development including tumour proliferation, metastasis and resistance. The overexpression and multi-factorial role of B7H3 in cancer makes B7H3 an attractive target for cancer therapies.

Thus, B7H3 has been considered in the development of cancer-cell specific chemotherapies, offering the opportunity to selectively target cancer cells while minimising damage to healthy non-cancer cells. A number of B7H3 targeting cancer therapies have been explored including, antibodies, CAR-T cell therapies and antibody-drug conjugates. However, there remains a need for anti-B7H3 antibodies and antibody-drug conjugates with favourable stability, pharmacokinetic properties, efficacy and/or safety profiles.

### SUMMARY OF THE DISCLOSURE

The present specification provides an antibody that binds to human B7H3 or antigen binding fragment thereof that binds human B7H3, wherein the antibody or antigen binding fragment thereof comprises (a) a heavy chain variable region (VH) comprising an HCDR1 of SEQ ID NO:1, an HCDR2 of SEQ ID NO:2 and an HCDR3 of SEQ ID NO:3, and (b) a light chain variable region (VL) comprising a LCDR1 of SEQ ID NO:4, a LCDR2 of SEQ ID NO:5 and a LCDR3 of SEQ ID NO:6.

In some embodiments, the VH comprises an amino acid sequence having at least 80%, at least 85%, at least 90%, at least 95%, at least 98%, at least 99%, or 100% sequence identity to the amino acid sequence of SEQ ID NO: 7. In some embodiments, the VL comprises an amino acid sequence having at least 80%, at least 85%, at least 90%, at least 95%, at least 98%, at least 99%, or 100% sequence identity to the amino acid sequence of SEQ ID NO:8.

In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 9 and/or a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody comprises a heavy chain sequence of SEQ ID NO: 9 and a light chain sequence of SEQ ID NO: 11.

In other embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 10 and/or a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody comprises a heavy chain sequence of SEQ ID NO: 10 and a light chain sequence of SEQ ID NO:11.

The present specification also provides a nucleic acid encoding the antibody or antigen binding fragment thereof as described herein.

The present specification also provides a vector comprising the nucleic acid as described herein.

The present specification also provides a host cell comprising the vector as described herein.

The present specification also provides a conjugate of Formula (I)

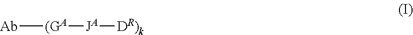

or a pharmaceutically acceptable salt thereof, wherein
Ab is an antibody that binds human B7H3, or an antigen binding fragment thereof that binds human B7H3,
k is an integer from about 1 to about 10,
   each G^{A} is independently a conjugation group conjugated to the antibody or antigen-binding fragment thereof,
   each D^{R} is independently a drug comprising a nitrogen atom N^{R},
   each J^{A} is independently a group of Formula (IA)

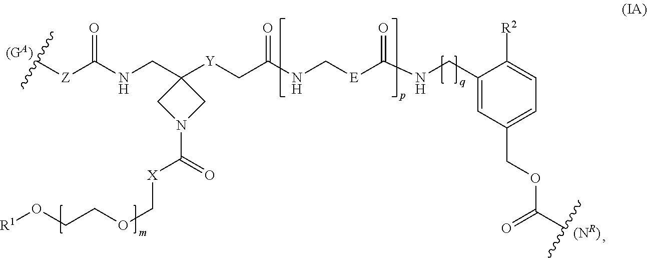

E is (CH₂)ₙ₁, wherein n1 is 0, 1, 2 or 3,
R¹ is C₁₋₄ alkyl or H,
R² is

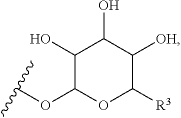

wherein R³ is CO₂H or CH₂OH,
X is (CH₂)ₙ₂,

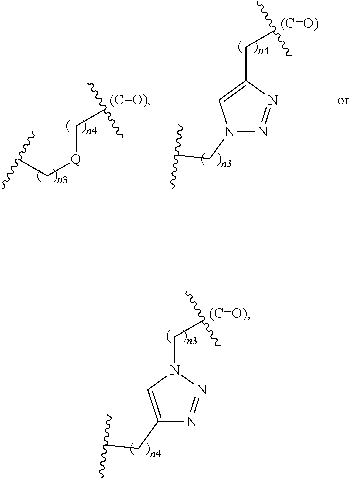

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

In some embodiments, Ab is as described herein.

In some embodiments, m is 9, 10, 11, 12 or 13, optionally wherein m is 11.

In some embodiments, R¹ is CH₃.

In some embodiments, R² is

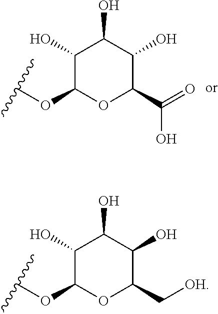 [SMILES: *O[C@@H]1O[C@H](C(=O)O)[C@@H](O)[C@H](O)[C@H]1O ; "or" ; *O[C@@H]1O[C@H](CO)[C@H](O)[C@H](O)[C@H]1O]

optionally wherein R² is

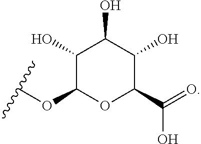 [SMILES: *O[C@@H]1O[C@H](C(=O)O)[C@@H](O)[C@H](O)[C@H]1O]

In some embodiments, E is CH₂.

In some embodiments, X is CH₂.

In some embodiments, Y is O.

In some embodiments, Z is (CH₂)₂.

In some embodiments, p is 1.

In some embodiments, q is 1.

In some embodiments, J^{A} is a group of Formula (IB)

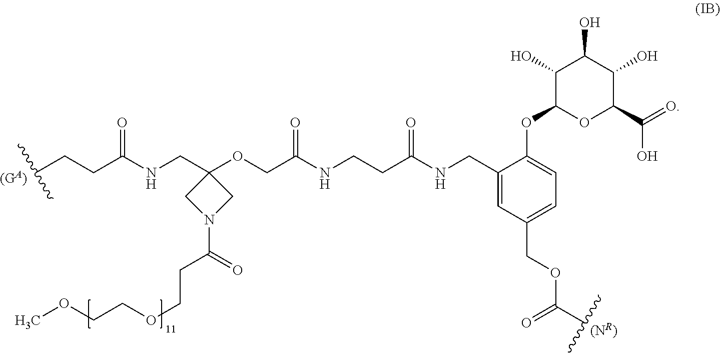

In some embodiments, G^{A} is selected from

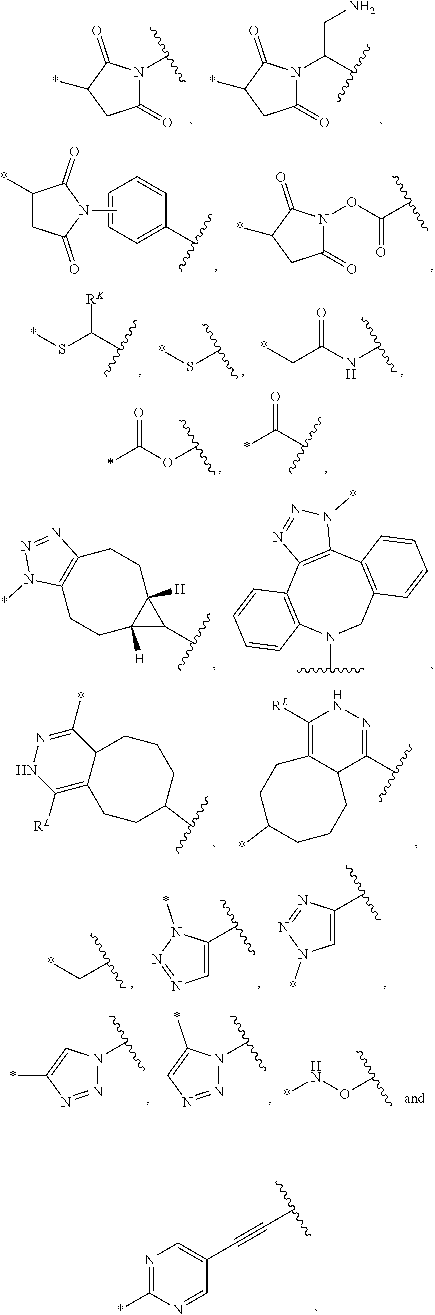

wherein R^{K} is H or CH₃, R^{L} is C₁₋₆ alkyl, and

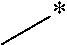 [SMILES: *C]

indicates the point of attachment to the antibody or antigen-binding fragment thereof.

In some embodiments, G^{A} is

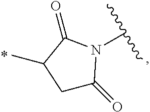 [SMILES: *N1C(=O)CC(C)C1=O ; ","]

wherein

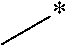 [SMILES: *C]

indicates the point of attachment to the antibody or antigen-binding fragment thereof.

In some embodiments, D^{R} is

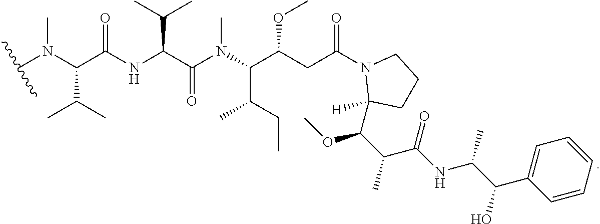 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, each G^{A}-J^{A}-D^{R} is a group of Formula (IC)

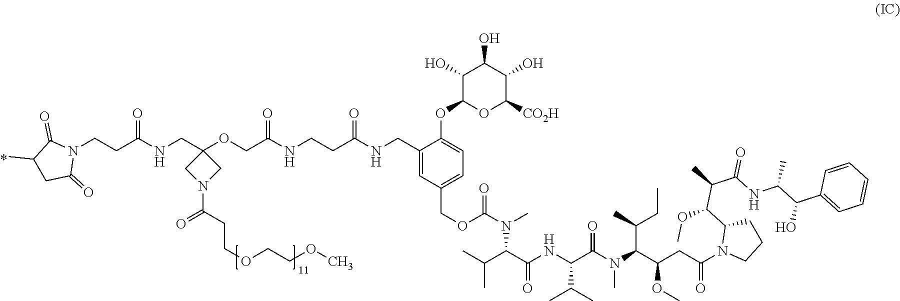

wherein

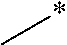 [SMILES: *C]

indicates the point of attachment to the antibody or antigen-binding fragment thereof.

In some embodiments, k is an integer from about 2 to about 6, optionally wherein k is an integer from about 2 to about 4. In some embodiments, k is about 2. In some embodiments, k is about 4.

The present specification provides a conjugate of Formula (I)

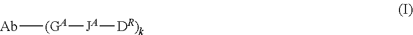

or a pharmaceutically acceptable salt thereof, wherein
Ab is an antibody that binds human B7H3 comprising a heavy chain sequence of SEQ ID NO: 9 and a light chain sequence of SEQ ID NO: 11,
k is about 2, and
each G^{A}J^{A}-D^{R} is a group of Formula (IC)

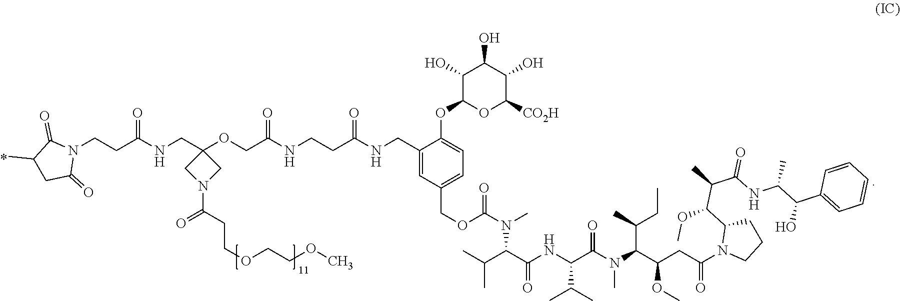

wherein [symbol] indicates the point of attachment to the antibody. In some embodiments, each G^{A}-J^{A}-D^{R} group is conjugated to a cysteine at position 174 of SEQ ID NO: 9.

The present specification also provides a conjugate of Formula (I)

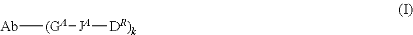

or a pharmaceutically acceptable salt thereof, wherein
Ab is an antibody that binds human B7H3 comprising a heavy chain sequence of SEQ ID NO: 10 and a light chain sequence of SEQ ID NO: 11,
k is about 4, and
each G^{A}-J^{A}-D^{R} is a group of Formula (IC)

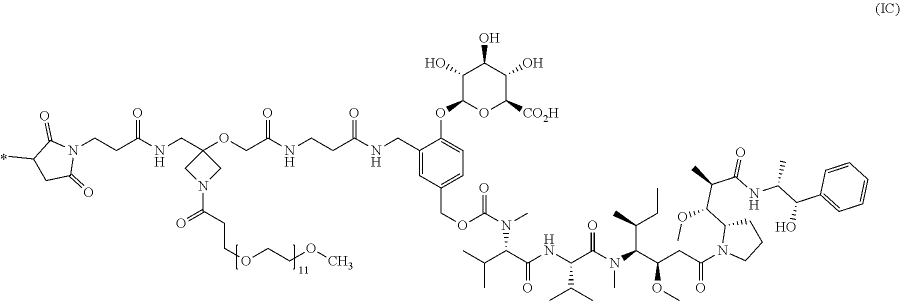

wherein [symbol] indicates the point of attachment to the antibody.

The present specification also provides a pharmaceutical composition comprising (i) the antibody or antigen binding fragment thereof as described herein, or the conjugate or a pharmaceutically acceptable salt thereof as described herein, and (ii) a pharmaceutically acceptable excipient.

Also provided is the antibody or antigen binding fragment thereof as described herein, the conjugate or pharmaceutically acceptable salt thereof as described herein or the pharmaceutical composition as described herein for use as a medicament.

Also provided is the antibody or antigen binding fragment thereof as described herein, the conjugate or pharmaceutically acceptable salt thereof as described herein or the pharmaceutical composition as described herein for use in treating cancer. In some embodiments, the cancer is a B7H3-expressing cancer. In some embodiments, the cancer is prostate cancer or squamous non-small cell lung cancer. In some embodiments, the antibody or antigen binding fragment thereof is used in combination with a further therapeutic agent.

The present specification also provides a method of treating a disease or disorder in a patient in need thereof comprising administering to the patient a therapeutically effective amount of the antibody or antigen binding fragment thereof as described herein or the conjugate or pharmaceutically acceptable salt thereof as described herein or the pharmaceutical composition as described herein. In some embodiments, the disease or disorder is cancer. In some embodiments, the cancer is a B7H3-expressing cancer. In some embodiments, the cancer is prostate cancer or squamous non-small cell lung cancer. In some embodiments, the antibody or antigen binding fragment thereof is used in combination with a further therapeutic agent.

The present specification also provides the use of an antibody or antigen binding fragment thereof as described herein or a conjugate or pharmaceutically acceptable salt thereof as described herein or a pharmaceutical composition as described herein, in the manufacture of a medicament for the treatment of cancer in a subject. In some embodiments, the cancer is a B7H3-expressing cancer. In some embodiments, the cancer is prostate cancer or squamous non-small cell lung cancer. In some embodiments, the antibody or antigen binding fragment thereof is used in combination with a further therapeutic agent.

The present specification also provides a kit comprising a conjugate or pharmaceutically acceptable salt thereof as described herein or a pharmaceutical composition as described herein, and instructions to administer the conjugate or pharmaceutically acceptable salt thereof or the pharmaceutical composition to a subject in need of treatment.

### BRIEF DESCRIPTION OF THE FIGURES

The present disclosure will now be described in more detail with reference to the attached Figures, in which:

FIG. 1A shows a representative UHPLC-RP chromatogram for ADC-1.

FIG. 1B shows a representative UHPLC-SEC chromatogram for ADC-1.

FIG. 2A shows the monomer content of ADC-2 starting antibody and ADC-2.

FIG. 2B shows the HIC retention times for the ADC-2 starting antibody and ADC-2.

FIG. 3 shows the ADC-2 starting antibody deconvoluted mass spectrometry chromatography.

FIG. 4 shows the ADC-2 deconvoluted mass spectrometry chromatography.

FIG. 5A shows the chromatographs for free drug analysis by UHPLC.

FIG. 5B shows a subset of the chromatographs of FIG. 5A.

FIGS. 6A-C show the stability of Comparative ADC-1 (FIG. 6A), ADC-1 (FIG. 6B) and ADC-2 (FIG. 6C) respectively in NSG mouse plasma.

FIG. 7 shows the MMAE payload delivery of Comparative ADC-1, ADC-1 and ADC-2 in a DU145 xenograft model.

FIG. 8 shows the internalisation of ADC-2 in C₄₋₂ cells.

FIG. 9 shows photostability data for mAb-1, mAb-2, ADC-1 and ADC-2 as measured by HPSEC.

FIG. 10 shows the anti-tumour efficacy of Comparative ADC-1, ADC-1 and ADC-2 in subcutaneous human prostate xenograft models.

FIGS. 11A-D show the anti-tumour efficacy of ADC-1 (FIGS. 11A and 11C) and ADC-2 (FIGS. 11B and 11D) respectively in patient derived xenograft models of prostate cancer.

FIGS. 12A-D show the anti-tumour efficacy of ADC-1 (FIGS. 12A and 12C) and ADC-2 (FIGS. 12B and 12D) respectively in patient derived xenograft models of squamous non-small cell lung cancer.

FIGS. 13A-B show the anti-tumour efficacy of ADC-1 (FIG. 13A) and ADC-2 (FIG. 13B) respectively in patient derived xenograft models of ovarian cancer.

FIGS. 14A-B show the anti-tumour efficacy of ADC-1 (FIG. 14A) and ADC-2 (FIG. 14B) respectively in patient derived xenograft models of endometrial cancer.

### DETAILED DESCRIPTION

This detailed description and its specific examples, while indicating embodiments, are intended for purposes of illustration only. Therefore, there is no limitation to the illustrative embodiments described in this specification. In addition, where the description has explicitly disclosed in isolation some individual features, any apparent combination of two or more such features is considered also to be disclosed, to the extent that such features or combinations are apparent and capable of being carried out based on the present specification as a whole in the light of the common general knowledge of a person skilled in the art, irrespective of whether such features or combinations of features solve any problems disclosed herein. In view of the foregoing description it will be evident to a person skilled in the art that various modifications may be made within the scope of the disclosure. All documents mentioned in this text are incorporated herein by reference.

### Definitions

Listed below are definitions of various terms used in the specification and claims.

As used herein, the terms “about” and “approximately”, when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided. As used herein, the term “alkyl” refers to a straight chained or branched non-aromatic hydrocarbon which is completely saturated. Alkyls may include straight chain(s) and/or branched chain(s).

In this specification the prefix Cₓ-C_{y} as used in terms such as “Cₓ-C_{y} alkyl” and the like where x and y are integers, indicates the numerical range of carbon atoms that are present in the group. Examples of suitable C₁-C₄ alkyl groups include methyl, ethyl, n-propyl, iso-propyl, n-butyl, i-butyl, sec-butyl and tert-butyl.

The term “cycloalkyl” refers to a completely saturated non-aromatic hydrocarbon ring system. Exemplary cycloalkyls include cyclopropyl, cyclobutyl, cyclopentyl, and cyclohexyl. Cycloalkyls include monocyclic cycloalkyls as well as spiro, fused and/or bridged polycyclic cycloalkyls such as bicyclic cycloalkyls. The term “(C₃-C₄)cycloalkyl” refers to a non-aromatic cyclic hydrocarbon ring having from three to four ring carbon atoms.

A “patient” refers to an animal in which the one or more active agents as described herein will have a therapeutic effect. In some embodiments, the patient is a human being. A “patient” includes warm blooded mammals, e.g., primates, dogs, cats, rabbits, rats, and mice.

The terms “polypeptide”, “peptide” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulphide bond formation, glycosylation, lipidation, acetylation, phosphorylation, or any other manipulation or modification, such as conjugation with a labelling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

The term “treating” or “treatment”, as used herein, unless otherwise indicated, means reversing, alleviating, inhibiting the progress of, or slowing or delaying the progression or reoccurrence of, the disorder or condition to which such term applies, or one or more symptoms of such disorder or condition.

The term “therapeutically effective amount” means any amount which, as compared to a corresponding subject who has not received such amount, results in improved treatment, healing, or amelioration of a disease, disorder, or side effect, or a decrease in the rate of advancement of a disease or disorder.

The use of “ [symbol] ” in formulas of this specification indicates the point of attachment to the antibody or antigen-binding fragment thereof. By way of illustration

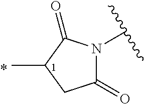 [SMILES: *N1C(=O)CC(C)C1=O]

indicates that that there is a covalent bond connecting the antibody, or antigen-binding fragment thereof, to the carbon atom marked 1.

For the avoidance of doubt, the use of “ [symbol] ” in formulas of this specification denotes the point of covalent attachment to a group, where the group is other than the antibody or antigen-binding fragment thereof.

Numeric ranges are inclusive of the numbers defining the range.

### Anti-B37H3 Antibody and Antigen Binding Fragment Thereof

The amino acid sequences of CDRs (according to Kabat and Chothia Combined system), heavy chain variable region (VH), light chain variable region (VL), whole heavy chain (HC) and light chain (LC) of the anti-human B37H-3 antibody or antigen binding fragment thereof of the present disclosure are indicated in Table 1.

TABLE 1

| | Sequence | SEQ ID NO |
|---|---|---|
| HCDR1 | SYAMS | 1 |
| HCDR2 | AISGSGGSTYYADSVKG | 2 |
| HCDR3 | DLGSGRGRHFDS | 3 |
| LCDR1 | TGSSSNIGAGYDVH | 4 |
| LCDR2 | GNSNRPS | 5 |
| LCDR3 | QSFDSSELWST | 6 |
| VH | EVQLLESGGGLVQPGGSLRLSCAASGFTFSSYAMSWVRQAPG | 7 |
| | KGLEWVSAISGSGGSTYYADSVKGRFTISRDNSKNTLYLQMNS | |
| | LRAEDTAVYYCARDLGSGRGRHFDSWGQGTLVTVSS | |
| VL | QAVLTQPPSVSGAPGQRVTISCTGSSSNIGAGYDVHWYQQLPG | 8 |
| | TAPKLLIYGNSNRPSGVPDRFSGSKSGTSASLAITGLQAEDEAD | |
| | YYCQSFDSSELWSTFGGGTKVTVL | |
| HC (L174C) | EVQLLESGGGLVQPGGSLRLSCAASGFTFSSYAMSWVRQAPG | 9 |
| | KGLEWVSAISGSGGSTYYADSVKGRFTISRDNSKNTLYLQMNS | |
| | LRAEDTAVYYCARDLGSGRGRHFDSWGQGTLVTVSSASTKGP | |
| | SVFPLAPSSKSTSGGTAALGCLVKDYFPEPVTVSWNSGALTSG | |
| | VHTFPAVCQSSGLYSLSSVVTVPSSSLGTQTYICNVNHKPSNTK | |
| | VDKRVEPKSCDKTHTCPPCPAPEFEGGPSVFLFPPKPKDTLMIS | |
| | RTPEVTCVVVDVSHEDPEVKFNWYVDGVEVHNAKTKPREEQY | |
| | NSTYRVVSVLTVLHQDWLNGKEYKCKVSNKALPASIEKTISKAK | |
| | GQPREPQVYTLPPSREEMTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| HC | EVQLLESGGGLVQPGGSLRLSCAASGFTFSSYAMSWVRQAPG | 10 |
| | KGLEWVSAISGSGGSTYYADSVKGRFTISRDNSKNTLYLQMNS | |
| | LRAEDTAVYYCARDLGSGRGRHFDSWGQGTLVTVSSASTKGP | |
| | SVFPLAPSSKSTSGGTAALGCLVKDYFPEPVTVSWNSGALTSG | |
| | VHTFPAVLQSSGLYSLSSVVTVPSSSLGTQTYICNVNHKPSNTK | |
| | VDKRVEPKSCDKTHTCPPCPAPEFEGGPSVFLFPPKPKDTLMIS | |
| | RTPEVTCVVVDVSHEDPEVKFNWYVDGVEVHNAKTKPREEQY | |
| | NSTYRVVSVLTVLHQDWLNGKEYKCKVSNKALPASIEKTISKAK | |
| | GQPREPQVYTLPPSREEMTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| LC | QAVLTQPPSVSGAPGQRVTISCTGSSSNIGAGYDVHWYQQLPG | 11 |
| | TAPKLLIYGNSNRPSGVPDRFSGSKSGTSASLAITGLQAEDEAD | |
| | YYCQSFDSSELWSTFGGGTKVTVLGQPKAAPSVTLFPPSSEEL | |
| | QANKATLVCLISDFYPGAVTVAWKADSSPVKAGVETTTPSKQS | |
| | NNKYAASSYLSLTPEQWKSHRSYSCQVTHEGSTVEKTVAPTEC | |
| | S | |

The nucleotide sequences of the whole heavy chain (HC) and light chain (LC) of the anti-human B37H-3 antibody or antigen binding fragment thereof of the present disclosure are indicated in Table 2.

TABLE 2

| | Sequence | SEQ ID NO |
|---|---|---|
| LC | CAAGCTGTTCTGACACAGCCTCCTAGTGTGTCTGGCGCTCC | 12 |
| | TGGCCAGAGAGTGACCATCTCTTGTACCGGCAGCAGCAGC | |
| | AATATCGGAGCCGGCTATGACGTGCACTGGTATCAGCAGC | |
| | TGCCTGGCACAGCCCCTAAACTGCTGATCTACGGCAACAG | |
| | CAACAGACCCAGCGGCGTGCCCGATAGATTTTCCGGCTCT | |
| | AAGAGCGGCACAAGCGCCAGCCTGGCTATTACTGGACTGC | |
| | AGGCCGAGGACGAGGCCGACTACTACTGCCAGAGCTTCGA | |
| | CAGCAGCGAGCTGTGGTCTACATTTGGCGGAGGCACCAAA | |
| | GTGACCGTGCTGGGACAACCTAAGGCgGCgCCTAGCGTGA | |
| | CCCTGTTTCCTCCAAGCAGCGAGGAACTGCAGGCTAACAA | |
| | GGCCACACTCGTGTGCCTGATCAGCGACTTCTATCCCGGC | |
| | GCTGTGACCGTGGCCTGGAAAGCTGATAGCTCTCCTGTGA | |
| | AGGCCGGCGTGGAAACCACCACACCTAGCAAGCAGAGCAA | |
| | CAACAAATACGCCGCCAGCAGCTACCTGAGCCTGACACCT | |
| | GAGCAGTGGAAGTCCCACAGATCCTACAGCTGCCAAGTGA | |
| | CCCACGAGGGCAGCACCGTGGAAAAGACAGTGGCCCCTAC | |
| | AGAGTGTTCA | |
| HC (L174C) | GAGGTGCAGCTGCTTGAATCTGGCGGAGGACTGGTTCAGC | 13 |
| | CTGGCGGATCTCTGAGACTGTCTTGTGCCGCCAGCGGCTT | |
| | CACCTTTAGCAGCTACGCCATGAGCTGGGTCCGACAGGCT | |
| | CCTGGCAAAGGCCTTGAATGGGTGTCCGCCATCTCTGGCA | |
| | GCGGCGGCAGCACATATTACGCCGATTCTGTGAAGGGCAG | |
| | ATTCACCATCAGCCGGGACAACAGCAAGAACACCCTGTACC | |
| | TCCAGATGAACAGCCTGAGAGCCGAGGACACCGCCGTGTA | |
| | CTATTGTGCCCGCGATCTCGGCTCTGGCAGAGGCAGACAC | |
| | TTTGATTCTTGGGGCCAGGGCACCCTGGTCACCGTTAGTTC | |
| | TGCGTCGACCAAGGGCCCATCCGTCTTCCCCCTGGCACCC | |
| | TCCTCCAAGAGCACCTCTGGGGGCACAGCGGCCCTGGGCT | |
| | GCCTGGTCAAGGACTACTTCCCCGAACCGGTGACGGTGTC | |
| | CTGGAACTCAGGCGCTCTGACCAGCGGCGTGCACACCTTC | |
| | CCGGCTGTCTGTCAGTCCTCAGGACTCTACTCCCTCAGCAG | |
| | CGTGGTGACCGTGCCCTCCAGCAGCTTGGGCACCCAGACC | |
| | TACATCTGCAACGTGAATCACAAGCCCAGCAACACCAAGGT | |
| | GGACAAGAGAGTTGAGCCCAAATCTTGTGACAAAACTCACA | |
| | CATGCCCACCGTGCCCAGCACCTGAATTCGAGGGGGGACC | |
| | GTCAGTCTTCCTCTTCCCCCCAAAACCCAAGGACACCCTCA | |
| | TGATCTCCCGGACCCCTGAGGTCACATGCGTGGTGGTGGA | |
| | CGTGAGCCACGAAGACCCTGAGGTCAAGTTCAACTGGTAC | |
| | GTGGACGGCGTGGAGGTGCATAATGCCAAGACAAAGCCGC | |
| | GGGAGGAGCAGTACAACAGCACGTACCGTGTGGTCAGCGT | |
| | CCTCACCGTCCTGCACCAGGACTGGCTGAATGGCAAGGAG | |
| | TACAAGTGCAAGGTCTCCAACAAAGCCCTCCCAGCCTCCAT | |
| | CGAGAAAACCATCTCCAAAGCCAAAGGGCAGCCCCGAGAA | |
| | CCACAGGTCTACACCCTGCCCCCATCCCGGGAGGAGATGA | |
| | CCAAGAACCAGGTCAGCCTGACCTGCCTGGTCAAAGGCTT | |
| | CTATCCCAGCGACATCGCCGTGGAGTGGGAGAGCAATGGG | |
| | CAGCCGGAGAACAACTACAAGACCACGCCTCCCGTGCTGG | |
| | ACTCCGACGGCTCCTTCTTCCTCTATAGCAAGCTCACCGTG | |
| | GACAAGAGCAGGTGGCAGCAGGGGAACGTCTTCTCATGCT | |
| | CCGTGATGCATGAGGCTCTGCACAACCACTACACGCAGAA | |
| | GAGCTTAAGCCTGTCTCCGGGTAAA | |
| HC | GAGGTGCAGCTGCTTGAATCTGGCGGAGGACTGGTTCAGC | 14 |
| | CTGGCGGATCTCTGAGACTGTCTTGTGCCGCCAGCGGCTT | |
| | CACCTTTAGCAGCTACGCCATGAGCTGGGTCCGACAGGCT | |
| | CCTGGCAAAGGCCTTGAATGGGTGTCCGCCATCTCTGGCA | |
| | GCGGCGGCAGCACATATTACGCCGATTCTGTGAAGGGCAG | |
| | ATTCACCATCAGCCGGGACAACAGCAAGAACACCCTGTACC | |
| | TCCAGATGAACAGCCTGAGAGCCGAGGACACCGCCGTGTA | |
| | CTATTGTGCCCGCGATCTCGGCTCTGGCAGAGGCAGACAC | |
| | TTTGATTCTTGGGGCCAGGGCACCCTGGTCACCGTTAGTTC | |
| | TGCGTCGACCAAGGGCCCATCCGTCTTCCCCCTGGCACCC | |
| | TCCTCCAAGAGCACCTCTGGGGGCACAGCGGCCCTGGGCT | |
| | GCCTGGTCAAGGACTACTTCCCCGAACCGGTGACGGTGTC | |
| | CTGGAACTCAGGCGCTCTGACCAGCGGCGTGCACACCTTC | |
| | CCGGCTGTCCTGCAGTCCTCAGGACTCTACTCCCTCAGCA | |
| | GCGTGGTGACCGTGCCCTCCAGCAGCTTGGGCACCCAGAC | |
| | CTACATCTGCAACGTGAATCACAAGCCCAGCAACACCAAGG | |
| | TGGACAAGAGAGTTGAGCCCAAATCTTGTGACAAAACTCAC | |
| | ACATGCCCACCGTGCCCAGCACCTGAATTCGAGGGGGGAC | |
| | CGTCAGTCTTCCTCTTCCCCCCAAAACCCAAGGACACCCTC | |
| | ATGATCTCCCGGACCCCTGAGGTCACATGCGTGGTGGTGG | |
| | ACGTGAGCCACGAAGACCCTGAGGTCAAGTTCAACTGGTA | |
| | CGTGGACGGCGTGGAGGTGCATAATGCCAAGACAAAGCCG | |
| | CGGGAGGAGCAGTACAACAGCACGTACCGTGTGGTCAGCG | |
| | TCCTCACCGTCCTGCACCAGGACTGGCTGAATGGCAAGGA | |
| | GTACAAGTGCAAGGTCTCCAACAAAGCCCTCCCAGCCTCCA | |
| | TCGAGAAAACCATCTCCAAAGCCAAAGGGCAGCCCCGAGA | |
| | ACCACAGGTCTACACCCTGCCCCCATCCCGGGAGGAGATG | |
| | ACCAAGAACCAGGTCAGCCTGACCTGCCTGGTCAAAGGCT | |
| | TCTATCCCAGCGACATCGCCGTGGAGTGGGAGAGCAATGG | |
| | GCAGCCGGAGAACAACTACAAGACCACGCCTCCCGTGCTG | |
| | GACTCCGACGGCTCCTTCTTCCTCTATAGCAAGCTCACCGT | |
| | GGACAAGAGCAGGTGGCAGCAGGGGAACGTCTTCTCATGC | |
| | TCCGTGATGCATGAGGCTCTGCACAACCACTACACGCAGAA | |
| | GAGCTTAAGCCTGTCTCCGGGTAAA | |

The term “Kabat numbering” and like terms are recognized in the art and refer to a system of numbering amino acid residues in the heavy and light chain variable regions of an antibody or an antigen binding fragment thereof. In some aspects, CDRS can be determined according to the Kabat numbering system (see, e.g. Kabat E A & Wu T T (1971) Ann NY Acad Sci 190: 382-391 and Kabat E A et al. (1991) Sequences of Proteins of Immunological Interest, Fifth Edition, U.S. Department of Health and Human Services, NIH Publication No. 91-3242). Using the Kabat numbering system, CDRS within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The terms “anti-B7H3 antibody”, “anti-human B7H3 antibody”, “human B7H3 antibody” and “antibody that binds to human B7H3” are used interchangeably herein to refer to an antibody that is capable of specifically binding to human B7H3. The extent of binding of a human B7H3 antibody to a non-B7H3 protein can be less than about 10% of the binding of the antibody to B7H3 as measured, e.g., using ForteBio or Biacore.

As used herein, the term “specifically binds” indicates that the antibody or antigen binding fragment thereof binds to an epitope via its antigen binding domain and that the binding entails some complementarity between the antigen binding domain and the epitope. Accordingly, in some aspects, an antibody that “specifically binds” to human B7H3 will not show any significant binding to other targets.

As used herein, the terms “variable region” or “variable domain” are used interchangeably and are common in the art. The terms “VL” and “VL domain” are used interchangeably to refer to the light chain variable region of an antibody. The terms “VH” and “VH domain” are used interchangeably to refer to the heavy chain variable region of an antibody.

As used herein, the term “heavy chain” when used in reference to an antibody can refer to any distinct type, e.g. alpha (a), delta (d), epsilon (e), gamma (g), and mu (m), based on the amino acid sequence of the constant domain, which give rise to IgA, IgD, IgE, IgG, and IgM classes of antibodies, respectively, including subclasses of IgG, e.g. IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g. kappa (K) or lambda (l) based on the amino acid sequence of the constant domains. Light chain amino acid sequences are well known in the art. In some aspects, the light chain is a human light chain.

Minor variations in the amino acid sequences of antibodies in the conjugates of the disclosure are contemplated as being encompassed by the present disclosure, providing that the variations in the amino acid sequence(s) maintain at least 80%, at least 90%, at least 95%, or at least 99% sequence identity to the antibody of the disclosure or antigen binding fragment thereof as defined herein, and the antibody or fragment can specifically bind human B7H3. In particular embodiments, the variations in the amino acid sequence(s) maintain at least 99% sequence identity to the antibody of the disclosure or antigen binding fragment thereof as defined herein.

In one embodiment, the antibody or antigen binding fragment described herein specifically binds human B7H3. Accordingly, provided herein is an antibody which specifically binds human B7H3. Also provided herein is an antigen binding fragment which specifically binds human B7H3. In one embodiment, the antibody or antigen binding fragment is fully human.

Provided herein is an antibody that binds to human B7H3 or antigen binding fragment thereof that binds human B7H3, wherein the antibody or antigen binding fragment thereof comprises (a) a heavy chain variable region (VH) comprising an HCDR1 of SEQ ID NO: 1, an HCDR2 of SEQ ID NO:2 and an HCDR3 of SEQ ID NO:3, and (b) a light chain variable region (VL) comprising a LCDR1 of SEQ ID NO:4, a LCDR2 of SEQ ID NO:5 and a LCDR3 of SEQ ID NO:6. In some embodiments, the antibody that binds to human B7H3 or antigen binding fragment thereof that binds human B7H3 is an antibody. In some embodiments, the antibody that binds to human B7H3 or antigen binding fragment thereof that binds human B7H3 is an antigen binding fragment.

In some embodiments, the VH comprises an amino acid sequence having at least 80%, at least 85%, at least 90%, at least 95%, at least 98%, at least 99%, or 100% sequence identity to the amino acid sequence of SEQ ID NO: 7. In some embodiments, the VH comprises an amino acid sequence having at least 90%, at least 95%, at least 98%, at least 99%, or 100% sequence identity to the amino acid sequence of SEQ ID NO: 7. In some embodiments, the VH comprises an amino acid sequence having at least 95%, at least 98%, at least 99%, or 100% sequence identity to the amino acid sequence of SEQ ID NO: 7. In some embodiments, the VH comprises an amino acid sequence of SEQ ID NO: 7. In some embodiments, the VH consists of an amino acid sequence of SEQ ID NO: 7.

In some embodiments, the VL comprises an amino acid sequence having at least 80%, at least 85%, at least 90%, at least 95%, at least 98%, at least 99%, or 100% sequence identity to the amino acid sequence of SEQ ID NO:8. In some embodiments, the VL comprises an amino acid sequence having at least 90%, at least 95%, at least 98%, at least 99%, or 100% sequence identity to the amino acid sequence of SEQ ID NO:8. In some embodiments, the VL comprises an amino acid sequence having at least 95%, at least 98%, at least 99%, or 100% sequence identity to the amino acid sequence of SEQ ID NO:8. In some embodiments, the VL comprises an amino acid sequence of SEQ ID NO:8. In some embodiments, the VL consists of an amino acid sequence of SEQ ID NO:8.

In some embodiments, the VH comprises an amino acid sequence of SEQ ID NO:7 and the VL comprises an amino acid sequence of SEQ ID NO:8. In some embodiments, the VH consists of an amino acid sequence of SEQ ID NO:7 and the VL consists of an amino acid sequence of SEQ ID NO:8. In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 9 and/or a light chain sequence comprising an amino acid sequence having at least 80%, at least 85%, at least 90%, at least 95%, at least 98%, at least 99%, or 100% sequence identity to the amino acid sequence of SEQ ID NO: 11.

In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 9 and a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody or antigen binding fragment thereof comprises a heavy chain sequence comprising an amino acid sequence having at least 90%, at least 95%, at least 98%, at least 99%, or 100% sequence identity to the amino acid sequence of SEQ ID NO: 9 and a light chain sequence comprising an amino acid sequence having at least 90%, at least 95%, at least 98%, at least 99%, or 100% sequence identity to the amino acid sequence of SEQ ID NO: 11. In some embodiments, the antibody comprises a heavy chain sequence of SEQ ID NO: 9 and a light chain sequence of SEQ ID NO: 11. In some embodiments, the antibody consists of a heavy chain sequence of SEQ ID NO: 9 and a light chain sequence of SEQ ID NO: 11.

In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 9 or a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 9. In some embodiments, the antibody or antigen binding fragment thereof comprises a heavy chain sequence comprising an amino acid sequence having at least 90%, at least 95%, at least 98%, at least 99%, or 100% sequence identity to the amino acid sequence of SEQ ID NO: 9. In some embodiments, the antibody or antigen binding fragment thereof comprises a heavy chain sequence comprising an amino acid sequence of SEQ ID NO: 9. In some embodiments, the antibody or antigen binding fragment thereof comprises a heavy chain sequence consisting of an amino acid sequence of SEQ ID NO: 9. In some embodiments, the antibody or antigen binding fragment thereof comprises a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody or antigen binding fragment thereof comprises a light chain sequence comprising an amino acid sequence having at least 90%, at least 95%, at least 98%, at least 99%, or 100% sequence identity to the amino acid sequence of SEQ ID NO: 11. In some embodiments, the antibody or antigen binding fragment thereof comprises a light chain sequence comprising an amino acid sequence of SEQ ID NO: 11. In some embodiments, the antibody or antigen binding fragment thereof comprises a light chain sequence consisting of an amino acid sequence of SEQ ID NO: 11.

In other embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 10 and/or a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody comprises a heavy chain sequence of SEQ ID NO: 10 and a light chain sequence of SEQ ID NO: 11.

In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 10 and a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody or antigen binding fragment thereof comprises a heavy chain sequence comprising an amino acid sequence having at least 90%, at least 95%, at least 98%, at least 99%, or 100% sequence identity to the amino acid sequence of SEQ ID NO: 10 and a light chain sequence comprising an amino acid sequence having at least 90%, at least 95%, at least 98%, at least 99%, or 100% sequence identity to the amino acid sequence of SEQ ID NO: 11. In some embodiments, the antibody comprises a heavy chain sequence of SEQ ID NO: 10 and a light chain sequence of SEQ ID NO: 11. In some embodiments, the antibody consists of a heavy chain sequence of SEQ ID NO: 10 and a light chain sequence of SEQ ID NO: 11.

In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 10 or a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 10. In some embodiments, the antibody or antigen binding fragment thereof comprises a heavy chain sequence comprising an amino acid sequence having at least 90%, at least 95%, at least 98%, at least 99%, or 100% sequence identity to the amino acid sequence of SEQ ID NO: 10. In some embodiments, the antibody or antigen binding fragment thereof comprises a heavy chain sequence comprising an amino acid sequence of SEQ ID NO: 10. In some embodiments, the antibody or antigen binding fragment thereof comprises a heavy chain sequence consisting of an amino acid sequence of SEQ ID NO: 10. In some embodiments, the antibody or antigen binding fragment thereof comprises a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody or antigen binding fragment thereof comprises a light chain sequence comprising an amino acid sequence having at least 90%, at least 95%, at least 98%, at least 99%, or 100% sequence identity to the amino acid sequence of SEQ ID NO: 11. In some embodiments, the antibody or antigen binding fragment thereof comprises a light chain sequence comprising an amino acid sequence of SEQ ID NO: 11. In some embodiments, the antibody or antigen binding fragment thereof comprises a light chain sequence consisting of an amino acid sequence of SEQ ID NO: 11.

Antibodies described herein may include variants in which amino acid residues from one species are substituted for the corresponding residue in another species, either at the conserved or non-conserved positions. In some embodiments, amino acid residues at non-conserved positions are substituted with conservative or non-conservative residues. In particular, conservative amino acid replacements are contemplated. In certain instances, the antibodies described herein may comprise heavy chains which have lost the C-terminal lysine i.e. the antibodies may have undergone C-terminal lysine clipping. For example, the C-terminal lysine as shown in SEQ ID NO: 9 and/or SEQ ID NO: 10 may be clipped from the heavy chain of the antibody, such as clipped from each heavy chain of the antibody.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in the antibodies in the conjugates of the disclosure does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, He, Phe or Val), (iii) a cysteine or proline is substituted for, or by, any other residue, or (iv) a residue having a bulky hydrophobic or aromatic side chain (e.g., Val, His, He or Trp) is substituted for, or by, one having a smaller side chain (e.g., Ala or Ser) or no side chain (e.g., Gly).

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the antibodies in the conjugates of the present disclosure. A limited number of non-conservative amino acids, amino acids that are not encoded by the genetic code, and unnatural amino acids may be substituted for amino acid residues. The antibodies in the conjugates of the present disclosure can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allothreonine, methyl-threonine, hydroxy-ethylcysteine, hydroxyethylhomo-cysteine, nitroglutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesising amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993). A limited number of non-conservative amino acids, amino acids that are not encoded by the genetic code, non-naturally occurring amino acids, and unnatural amino acids may be substituted for amino acid residues of antibodies in the conjugates of the present disclosure.

Essential amino acids in the antibodies in the conjugates of the present disclosure can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the antibodies in the conjugates of the present disclosure.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomising two or more positions in a polypeptide, selecting for functional polypeptide, and then sequencing the mutagenised polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimise alignment of two or more sequences. Sequence comparisons and the determination of percent identity between two or more sequences can be carried out using specific mathematical algorithms, such as BLAST, which will be familiar to a skilled person.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Percent sequence identity can be determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimise the alignment scores using a gap opening penalty of 10, a gap extension penalty of 1, and the “blosum 62” scoring matrix of Henikoff and Henikoff (ibid.) as shown below (amino acids are indicated by the standard one-letter codes).

In some embodiments, the variable domains in both the heavy and light chains of an antibody or antigen binding fragment thereof in the conjugate are altered by at least partial framework region replacement and sequence changing.

In some embodiments, the antibody or antigen binding fragment thereof in the conjugate can include, in addition to a VH and a VL, a heavy chain constant region or fragment thereof. In some embodiments, the heavy chain constant region is a human heavy chain constant region, e.g., a human IgG constant region, e.g., a human IgGl constant region.

In some embodiments, the antigen binding fragment as described herein is a Fab fragment. The antigen binding fragment according to the disclosure can also be a Fab′, an Fv, an scFv, an Fd, a V NAR domain, an IgNAR, an intrabody, an IgG CH₂, a minibody, a single domain antibody, an Fcab, an scFv-Fc, F(ab′)2, a di-scFv, a bi-specific T-cell engager (BiTE®), a F(ab′)3, a tetrabody, a triabody, a diabody, a DVD-Ig, an (scFv)2, a mAb2 or a DARPin.

The terms “Fab fragment” and “Fab” are used interchangeably herein and contain a single light chain (e.g. a constant domain CL and a VL) and a single heavy chain (e.g. a constant domain CH1 and a VH).

The heavy chain of a Fab fragment is not capable of forming a disulphide bond with another heavy chain.

A “Fab1 fragment” contains a single light chain and a single heavy chain but in addition to the CH1 and the VH, a “Fab1 fragment” contains the region of the heavy chain between the CH1 and CH₂ domains that is required for the formation of an inter-chain disulphide bond. Thus, two “Fab1 fragments” can associate via the formation of a disulphide bond to form a F(ab′)2 molecule. A “F(ab′)2 fragment” contains two light chains and two heavy chains. Each chain includes a portion of the constant region necessary for the formation of an inter-chain disulphide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is an antibody fragment containing the VH and VL domain of an antibody, linked together to form a single chain. A polypeptide linker is commonly used to connect the VH and VL domains of the scFv.

A “tandem scFv”, also known as a TandAb®, is a single-chain Fv molecule formed by covalent bonding of two scFvs in a tandem orientation with a flexible peptide linker.

A “bi-specific T cell engager” (BiTE®) is a fusion protein consisting of two single-chain variable fragments (scFvs) on a single peptide chain. One of the scFvs binds to T cells via the CD3 receptor, and the other to a tumour cell antigen.

A “diabody” is a small bivalent and bispecific antibody fragment comprising a heavy chain variable domain (VH) connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

A “DARPin” is a bispecific ankyrin repeat molecule. DARPins are derived from natural ankyrin proteins, which can be found in the human genome and are one of the most abundant types of binding proteins. A DARPin library module is defined by natural ankyrin repeat protein sequences, using 229 ankyrin repeats for the initial design and another 2200 for subsequent refinement. The modules serve as building blocks for the DARPin libraries. The library modules resemble human genome sequences. A DARPin is composed of 4 to 6 modules. Because each module is approx. 3.5 kDa, the size of an average DARPin is 16-21 kDa. Selection of binders is done by ribosome display, which is completely cell-free and is described in He M. and Taussig M J., Biochem Soc Trans. 2007, November; 35(Pt 5):962-5.

In some embodiments, the antibodies disclosed herein can be modified to comprise alterations or modifications to one or more of the three heavy chain constant domains (CH1, CH2 or CH3) and/or to the light chain constant domain (CL). In some embodiments, a modified constant region wherein one or more domains are partially or entirely deleted are contemplated. In some embodiments, a modified antibody in the conjugate will comprise domain deleted constructs or variants wherein the entire CH2 domain has been removed (ACH2 constructs). In some embodiments, the omitted constant region domain can be replaced by a short amino acid spacer (e.g., 10 residues) that provides some of the molecular flexibility typically imparted by the absent constant region. The deletion or inactivation (through point mutations or other means) of a constant region domain can reduce Fc receptor binding of the circulating modified antibody. In other cases, it can be that the constant region modifications moderate complement binding and thus reduce the serum half-life and nonspecific association of a conjugated cytotoxin. Yet other modifications of the constant region can be used to eliminate disulphide linkages or oligosaccharide moieties that allow for enhanced localisation due to increased antigen specificity or antibody flexibility. In some embodiments, the antibody or antigen binding fragment thereof in the conjugate has no antibody-dependent cellular cytotoxicity (ADCC) activity and/or no complement-dependent cytotoxicity (CDC) activity.

Besides the deletion of whole constant region domains, an antibody or antigen binding fragment thereof, e.g. in the conjugate provided herein, can be modified by the partial deletion or substitution of a few or even a single amino acid in a constant region. For example, the mutation of a single amino acid in selected areas of the CH2 domain can be enough to substantially reduce Fc binding and thereby increase tumour localisation. Similarly one or more constant region domains that control the effector function (e.g., complement C1Q binding) can be fully or partially deleted. Such partial deletions of the constant regions can improve selected characteristics of the antibody or antigen binding fragment thereof, e.g. in the conjugate, (e.g., serum half-life) while leaving other desirable functions associated with the subject constant region domain intact. Moreover, the constant regions of the antibody and antigen binding fragment thereof, e.g. in the conjugate, can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen-binding fragment thereof, e.g. in the conjugate. In some embodiments, there may be an addition of one or more amino acids to the constant region to enhance desirable characteristics such as decreasing or increasing effector function or provide for more cytotoxin or carbohydrate attachment. In some embodiments, it can be desirable to insert or replicate specific sequences derived from selected constant region domains. In some embodiments, a heavy chain constant region or fragment thereof, e.g., a human IgG constant region or fragment thereof, can include one or more amino acid substitutions relative to a wild-type IgG constant domain wherein the modified IgG has an increased half-life compared to the half-life of an IgG having the wildtype IgG constant domain. For example, the IgG constant domain can contain one or more amino acid substitutions of amino acid residues at positions 251-257, 285-290, 308-314, 385-389, and 428-436, wherein the amino acid position numbering is according to the EU index as set forth in Kabat.

In some embodiments the IgG constant domain can contain one or more of a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Serine (S), Histidine (H), Phenylalanine (F), or Tyrosine. In some embodiments, the antibodies or antigen binding fragments thereof in the ADC comprise a YTE mutant. The terms “YTE” or “YTE mutant” refer to a mutation in IgGl Fc that results in an increase in the binding to human FcRn and improves the serum half-life of the antibody having the mutation. A YTE mutant comprises a combination of three mutations, M252Y/S254T/T256E (EU numbering Kabat et al. (1991) Sequences of Proteins of Immunological Interest, U.S. Public Health Service, National Institutes of Health, Washington, D.C.), introduced into the heavy chain of an IgGl. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Biol. Chem. 281:23514-24 (2006); Robbie et al., (2013) Antimicrob. Agents Chemother. 57, 6147-6153). See also U.S. Pat. No. 7,083,784, which is hereby incorporated by reference in its entirety.

### Binding Measurement

Any suitable method may be used to determine whether an antibody binds to human B7H3. Such a method may comprise surface plasmon resonance (SPR), bio-layer interferometry, or an ELISA to determine specificity of antibodies. An antibody may be said to bind its antigen if the level of binding to antigen is at least 2.5 fold greater, e.g., at least 10 fold greater, than binding to a control antigen. Binding between an antibody and its cognate antigen is often referred to as specific binding. Precise identification of the residues bound by an antibody can usually be obtained using x-ray crystallography. This technique may be used to determine that an antibody described herein binds one or more residues of human B7H3.

Ability of an antibody to bind its target antigen, and the specificity and affinity of that binding (K_{D}, Kd and/or Ka) can be determined by any routine method in the art, e.g. using surface plasmon resonance (SPR), such as by Biacore™ (Cytiva Life Sciences) or using the ProteOn XPR36™ (Bio-Rad®), using KinExA® (Sapidyne Instruments, Inc), or using ForteBio Octet (Pall ForteBio Corp.).

“Binding affinity” generally refers to the strength of the sum total of non-covalent interactions between a single binding site of a molecule (e.g. an antibody or antigen binding fragment thereof) and its binding partner (e.g. an antigen). Unless indicated otherwise, as used herein, “binding affinity” refers to intrinsic binding affinity which reflects a 1:1 interaction between members of a binding pair (e.g. antibody or antigen binding fragment thereof and antigen). The affinity of a molecule X for its partner Y can generally be represented by the equilibrium dissociation constant (K_{D}). Affinity can be measured and/or expressed in a number of ways known in the art, including, but not limited to, equilibrium dissociation constant (K_{D}), and equilibrium association constant (KA). The K_{D} is calculated from the quotient of k_{off}/kₒₙ, whereas KA is calculated from the quotient of kₒₙ/k_{off}. kₒₙ refers to the association rate constant of, e.g, an antibody or antigen binding fragment thereof to an antigen, and k_{off} refers to the dissociation of, e.g, an antibody or antigen binding fragment thereof from an antigen. The kₒₙ and k_{off} can be determined by techniques known to one of ordinary skill in the art, such as BIAcore® or KinExA.

The term “K_{D}”, as used herein, is intended to refer to the equilibrium dissociation constant of a particular antibody-antigen interaction. Affinity of antibody-antigen binding may be determined, e.g., by SPR. Affinity may also be determined by bio-layer interferometry. Examples of affinity determination are provided in Example 3 herein. In some examples, an antibody or antigen binding fragment in an ADC described herein may bind to human B7H3 with an affinity (K_{D}) of 1 mM or less, such as less than 90 nM, less than 80 nM, less than 70 nM, less than 60 nM or less than 50 nM, as determined by SPR. In some examples, an antibody in an ADC described herein may additionally or alternatively bind to cyno B7H3 with an affinity (K_{D}) of 1 mM or less, such as less than 90 nM, less than 80 nM, less than 70 nM or less than 60 nM as determined by SPR and cyno B7H3 with an affinity (K_{D}) of less than 20 nM, less than 15 nM or less than 12 nM.

In one example, the SPR is carried out at 37° C. In brief, the affinity of the antibody can be determined using SPR by:
1. Coupling mouse anti-human (or other relevant human, rat or non-human vertebrate antibody constant region species-matched) IgG to a biosensor chip (e.g. dextran-coated gold chip) such as by primary amine coupling. Thus, an anti-Fc antibody may be covalently immobilised on the chip surface using amine coupling.
2. Exposing the mouse anti-human IgG (or other matched species antibody) to the test antibody (e.g., in human IgG format) to capture the test antibody on the chip;
3. Passing the test antigen over the chip's capture surface at a series of concentrations and a 0 nM (i.e. buffer alone) control run. The buffer may optionally be 0.01 M HEPES (4-(2-hydroxyethyl)-1-piperazineethanesulfonic acid), 0.15 M NaCl and 0.05% v/v surfactant P20 in aqueous solution, buffered to pH 7.4; and
4. Determining affinity of binding of test antibody to test antigen using surface plasmon resonance. K_{D}, Ka and Kd.

SPR can be carried out using any standard SPR apparatus, such as by Biacore T200 or using the ProteOn XPR36™ (Bio-Rad®).

Regeneration of the capture surface can be carried out with 3 M magnesium chloride solution. This removes the captured test antibody and allows the surface to be used for another interaction. The binding data can be fitted to 1:1 model inherent using standard techniques, e.g. using analysis software such as Biacore Insight Evaluation Software.

### Nucleic Acids, Vectors, Host Cells

The present specification also provides a nucleic acid encoding the antibody or antigen binding fragment thereof as described herein.

In some embodiments, the nucleic acid encodes a heavy chain of any one of the antibodies described herein. In some embodiments, the nucleic acid encodes a light chain of any one of the antibodies described herein.

In some embodiments, the heavy chain nucleic acid sequence is at least 70% identical to SEQ ID NO: 13. In some embodiments, the heavy chain nucleic acid sequence is at least 80% identical to SEQ ID NO: 13. In some embodiments, the heavy chain nucleic acid sequence is at least 90% identical to SEQ ID NO: 13. In some embodiments, the heavy chain nucleic acid sequence is at least 95% identical to SEQ ID NO: 13. In some embodiments, the heavy chain nucleic acid sequence is at least 96% identical to SEQ ID NO: 13. In some embodiments, the heavy chain nucleic acid sequence is at least 97% identical to SEQ ID NO: 13. In some embodiments, the heavy chain nucleic acid sequence is at least 98% identical to SEQ ID NO: 13. In some embodiments, the heavy chain nucleic acid sequence is at least 99% identical to SEQ ID NO: 13. In some embodiments, the heavy chain nucleic acid sequence is at least 99.5% identical to SEQ ID NO: 13. In some embodiments, the heavy chain nucleic acid sequence consists of SEQ ID NO: 13.

In other embodiments, the heavy chain nucleic acid sequence is at least 70% identical to SEQ ID NO: 14. In some embodiments, the heavy chain nucleic acid sequence is at least 80% identical to SEQ ID NO: 14. In some embodiments, the heavy chain nucleic acid sequence is at least 90% identical to SEQ ID NO: 14. In some embodiments, the heavy chain nucleic acid sequence is at least 95% identical to SEQ ID NO: 14. In some embodiments, the heavy chain nucleic acid sequence is at least 96% identical to SEQ ID NO: 14. In some embodiments, the heavy chain nucleic acid sequence is at least 97% identical to SEQ ID NO: 14. In some embodiments, the heavy chain nucleic acid sequence is at least 98% identical to SEQ ID NO: 14. In some embodiments, the heavy chain nucleic acid sequence is at least 99% identical to SEQ ID NO: 14. In some embodiments, the heavy chain nucleic acid sequence is at least 99.5% identical to SEQ ID NO: 14. In some embodiments, the heavy chain nucleic acid sequence consists of SEQ ID NO: 14.

In some embodiments, the light chain nucleic acid sequence is at least 70% identical to SEQ ID NO: 12. In some embodiments, the heavy chain nucleic acid sequence is at least 80% identical to SEQ ID NO: 12. In some embodiments, the heavy chain nucleic acid sequence is at least 90% identical to SEQ ID NO: 12. In some embodiments, the heavy chain nucleic acid sequence is at least 95% identical to SEQ ID NO: 12. In some embodiments, the heavy chain nucleic acid sequence is at least 96% identical to SEQ ID NO: 12. In some embodiments, the heavy chain nucleic acid sequence is at least 97% identical to SEQ ID NO: 12. In some embodiments, the heavy chain nucleic acid sequence is at least 98% identical to SEQ ID NO: 12. In some embodiments, the heavy chain nucleic acid sequence is at least 99% identical to SEQ ID NO: 12. In some embodiments, the heavy chain nucleic acid sequence is at least 99.5% identical to SEQ ID NO: 12. In some embodiments, the light chain nucleic acid sequence consists of SEQ ID NO: 12.

In some embodiments, the heavy chain nucleic acid sequence is at least 70% identical to SEQ ID NO: 13 and the light chain nucleic acid sequence is at least 70% identical to SEQ ID NO: 12. In some embodiments, the heavy chain nucleic acid sequence is at least 80% identical to SEQ ID NO: 13 and the light chain nucleic acid sequence is at least 80% identical to SEQ ID NO: 12. In some embodiments, the heavy chain nucleic acid sequence is at least 90% identical to SEQ ID NO: 13 and the light chain nucleic acid sequence is at least 90% identical to SEQ ID NO: 12. In some embodiments, the heavy chain nucleic acid sequence is at least 95% identical to SEQ ID NO: 13 and the light chain nucleic acid sequence is at least 95% identical to SEQ ID NO: 12. In some embodiments, the heavy chain nucleic acid sequence is at least 96% identical to SEQ ID NO: 13 and the light chain nucleic acid sequence is at least 96% identical to SEQ ID NO: 12. In some embodiments, the heavy chain nucleic acid sequence is at least 97% identical to SEQ ID NO: 13 and the light chain nucleic acid sequence is at least 97% identical to SEQ ID NO: 12. In some embodiments, the heavy chain nucleic acid sequence is at least 98% identical to SEQ ID NO: 13 and the light chain nucleic acid sequence is at least 98% identical to SEQ ID NO: 12. In some embodiments, the heavy chain nucleic acid sequence is at least 99% identical to SEQ ID NO: 13 and the light chain nucleic acid sequence is at least 99% identical to SEQ ID NO: 12. In some embodiments, the heavy chain nucleic acid sequence is at least 99.5% identical to SEQ ID NO: 13 and the light chain nucleic acid sequence is at least 99.5% identical to SEQ ID NO: 12. In some embodiments, the heavy chain nucleic acid sequence consists of SEQ ID NO: 13 and the light chain nucleic acid sequence consists of SEQ ID NO: 12.

In some embodiments, the heavy chain nucleic acid sequence is at least 70% identical to SEQ ID NO: 14 and the light chain nucleic acid sequence is at least 70% identical to SEQ ID NO: 12. In some embodiments, the heavy chain nucleic acid sequence is at least 80% identical to SEQ ID NO: 14 and the light chain nucleic acid sequence is at least 80% identical to SEQ ID NO: 12. In some embodiments, the heavy chain nucleic acid sequence is at least 90% identical to SEQ ID NO: 14 and the light chain nucleic acid sequence is at least 90% identical to SEQ ID NO: 12. In some embodiments, the heavy chain nucleic acid sequence is at least 95% identical to SEQ ID NO: 14 and the light chain nucleic acid sequence is at least 95% identical to SEQ ID NO: 12. In some embodiments, the heavy chain nucleic acid sequence is at least 96% identical to SEQ ID NO: 14 and the light chain nucleic acid sequence is at least 96% identical to SEQ ID NO: 12. In some embodiments, the heavy chain nucleic acid sequence is at least 97% identical to SEQ ID NO: 14 and the light chain nucleic acid sequence is at least 97% identical to SEQ ID NO: 12. In some embodiments, the heavy chain nucleic acid sequence is at least 98% identical to SEQ ID NO: 14 and the light chain nucleic acid sequence is at least 98% identical to SEQ ID NO: 12. In some embodiments, the heavy chain nucleic acid sequence is at least 99% identical to SEQ ID NO: 14 and the light chain nucleic acid sequence is at least 99% identical to SEQ ID NO: 12. In some embodiments, the heavy chain nucleic acid sequence is at least 99.5% identical to SEQ ID NO: 14 and the light chain nucleic acid sequence is at least 99.5% identical to SEQ ID NO: 12. In some embodiments, the heavy chain nucleic acid sequence consists of SEQ ID NO: 14 and the light chain nucleic acid sequence consists of SEQ ID NO: 12. In some embodiments, the nucleic acid is an isolated and purified nucleic acid.

The present specification also provides a vector comprising the nucleic acid as described herein. In some embodiments, the vector may be a CHO vector. In some embodiments, the vector may be a HEK293 vector.

The present specification also provides a host cell comprising the vector as described herein. In some embodiments, the host cells are eukaryotic cells, e.g., mammalian cells, preferably CHO cells (e.g., CHO cells grown in suspension culture).

As used herein, the term “host cell” can be any type of cell, e.g. a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

### Antibody-Drug Conjugates (ADC)

Antibody drug conjugates (ADCs) are an established method to deliver a drug to a biological target. It is possible to use an enzymatically cleavable linker to release free drug at the target, so that the free drug can have a therapeutic effect. Linkers used in the ADCs disclosed herein are β-glucuronidase-cleavable linkers. Through transcriptomic and proteomic profiling of various solid tumour cell lines, β-glucuronidase expression has been identified as being upregulated and typically localised to the lysosome.

The “antibody-drug conjugate” (ADC) of the disclosure comprises an anti-B7H3 antibody or antigen binding fragment thereof as described herein, wherein the anti-B7H3 antibody or antigen binding fragment is conjugated to a therapeutic drug with a 0-glucuronidase-cleavable linker.

The ADCs of the present disclosure have a number of desirable qualities. A conjugate of Formula (I), or a pharmaceutically acceptable salt thereof, may undergo enzymatic cleavage to release a free drug, e.g. the free drug form of the drug is monomethyl auristatin E (MMAE) ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide). Accordingly, in some embodiments, a conjugate of Formula (I), or a pharmaceutically acceptable salt thereof, undergoes enzymatic cleavage to release monomethyl auristatin E (MMAE) ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide).

Conjugates of Formula (I), or pharmaceutically acceptable salts thereof, may exhibit improved efficacy and/or advantageous physical properties (for example, higher stability, lower lipophilicity, higher aqueous solubility, higher permeability and/or lower plasma protein binding), and/or favourable toxicity profiles (for example reduced off target toxicity), and/or favourable metabolic or pharmacokinetic profiles, in comparison with other conjugates. Conjugates of Formula (I), or pharmaceutically acceptable salts thereof, may exhibit improved colloidal stability in comparison with other conjugates. As such, conjugates of Formula (I) or pharmaceutically acceptable salts thereof as described herein may be especially suitable for use in therapy, such as the treatment of cancer.

As described herein, conjugates of the present disclosure may undergo slower cleavage in the presence of β-glucuronidase relative to other conjugates. Without wishing to be bound by theory, it is believed that slower cleavage of the linker to release a free drug may result in improved tolerability of the conjugate by reducing the proportion of free drug released in off-target tissue.

The conjugates of the present disclosure have an advantageous pharmacokinetic profile. In particular, the conjugates of the present disclosure have a high plasma exposure compared to vcMMAE ADC control (Example 6). The conjugates of the present disclosure also achieve sustained tumor drug exposure (Example 7).

The present specification also provides a conjugate of Formula (I)

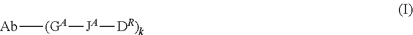

or a pharmaceutically acceptable salt thereof, wherein
Ab is an antibody that binds human B7H3, or an antigen binding fragment thereof that binds human B7H3,
k is an integer from about 1 to about 10,
each G^{A} is independently a conjugation group conjugated to the antibody or antigen-binding fragment thereof,
each D^{R} is independently a drug comprising a nitrogen atom N^{R},
each J^{A} is independently a group of Formula (IA)

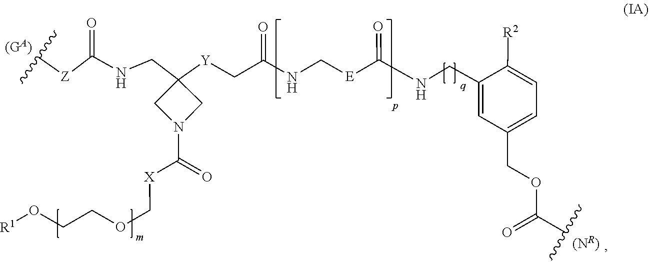

E is (CH₂)ₙ₁, wherein n1 is 0, 1, 2 or 3,
R¹ is C₁₋₄ alkyl or H,
R² is

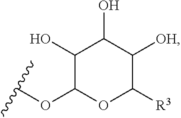

wherein R³ is CO₂H or CH₂OH,
X is (CH₂)ₙ₂,

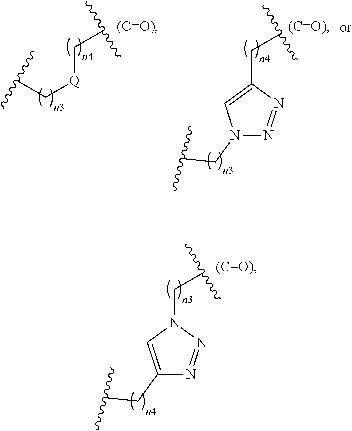

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

### Antibody or Antigen Binding Fragment Thereof

In some embodiments, the antibody or antigen binding fragment thereof comprises (a) a heavy chain variable region (VH) comprising an HCDR1 of SEQ ID NO: 1, an HCDR2 of SEQ ID NO:2 and an HCDR3 of SEQ ID NO:3, and (b) a light chain variable region (VL) comprising a LCDR1 of SEQ ID NO:4, a LCDR2 of SEQ ID NO:5 and a LCDR3 of SEQ ID NO:6.

Accordingly, provided herein is a conjugate of Formula (I)

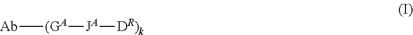

or a pharmaceutically acceptable salt thereof, wherein
Ab is an antibody that binds human B7H3, or an antigen binding fragment thereof that binds human B7H3, wherein the antibody or antigen binding fragment thereof comprises (a) a heavy chain variable region (VH) comprising an HCDR1 of SEQ ID NO:1, an HCDR2 of SEQ ID NO:2 and an HCDR3 of SEQ ID NO:3, and (b) a light chain variable region (VL) comprising a LCDR1 of SEQ ID NO:4, a LCDR2 of SEQ ID NO:5 and a LCDR3 of SEQ ID NO:6,
k is an integer from about 1 to about 10,
each G^{A} is independently a conjugation group conjugated to the antibody or antigen-binding fragment thereof,
each D^{R} is independently a drug comprising a nitrogen atom N^{R},
each J^{A} is independently a group of Formula (IA)

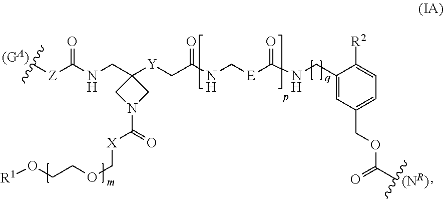

E is (CH₂)ₙ₁, wherein n1 is 0, 1, 2 or 3,
R¹ is C₁₋₄ alkyl or H,
R² is

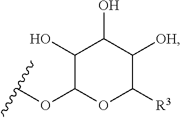

wherein R³ is CO₂H or CH₂OH,
X is (CH₂)ₙ₂,

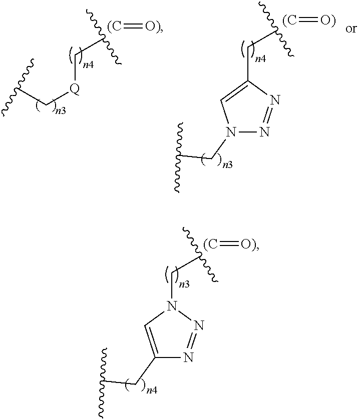

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

In some embodiments, the VH comprises an amino acid sequence having at least 80%, at least 85%, at least 90%, at least 95%, at least 98%, at least 99%, or 100% sequence identity to the amino acid sequence of SEQ ID NO: 7. In some embodiments, the VL comprises an amino acid sequence having at least 80%, at least 85%, at least 90%, at least 95%, at least 98%, at least 99%, or 100% sequence identity to the amino acid sequence of SEQ ID NO:8. In some embodiments, the VH consists of an amino acid sequence having at least 80%, at least 85%, at least 90%, at least 95%, at least 98%, at least 99%, or 100% sequence identity to the amino acid sequence of SEQ ID NO: 7. In some embodiments, the VL consists of an amino acid sequence having at least 80%, at least 85%, at least 90%, at least 95%, at least 98%, at least 99%, or 100% sequence identity to the amino acid sequence of SEQ ID NO:8. In embodiments, the VH comprises an amino acid sequence of SEQ ID NO: 7 and the VL comprises an amino acid sequence of SEQ ID: 8.

In some embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 9 and/or a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody comprises a heavy chain sequence of SEQ ID NO: 9 and a light chain sequence of SEQ ID NO: 11. In some embodiments, the antibody or antigen binding fragment thereof consists of a heavy chain sequence consisting of an amino acid sequence having at least 80%, at least 85%, at least 90%, at least 95%, at least 98%, at least 99%, or 100% sequence identity to the amino acid sequence of SEQ ID NO: 9 and/or a light chain sequence consisting of an amino acid sequence having at least 80%, at least 85%, at least 90%, at least 95%, at least 98%, at least 99%, or 100% sequence identity to the amino acid sequence of SEQ ID NO: 11. In some embodiments, the antibody consists of a heavy chain sequence of SEQ ID NO: 9 and a light chain sequence of SEQ ID NO: 11.

Accordingly, provided herein is a conjugate of Formula (I)

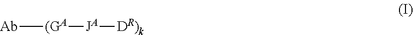

or a pharmaceutically acceptable salt thereof, wherein
Ab is an antibody that binds human B7H3, or an antigen binding fragment thereof that binds human B7H3, wherein the antibody or antigen binding fragment thereof comprise a VH comprising an amino acid sequence of SEQ ID NO: 7, and a VL comprising an amino acid sequence of SEQ ID NO: 8,
k is an integer from about 1 to about 10,
each G^{A} is independently a conjugation group conjugated to the antibody or antigen binding fragment thereof,
each D^{R} is independently a drug comprising a nitrogen atom N^{R},
each J^{A} is independently a group of Formula (IA)

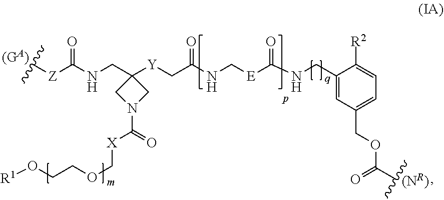

E is (CH₂)ₙ₁, wherein n1 is 0, 1, 2 or 3,
R¹ is C₁₋₄ alkyl or H,
R² is

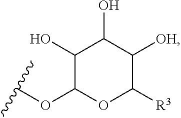

wherein R³ is CO₂H or CH₂OH,
X is (CH₂)ₙ₂,

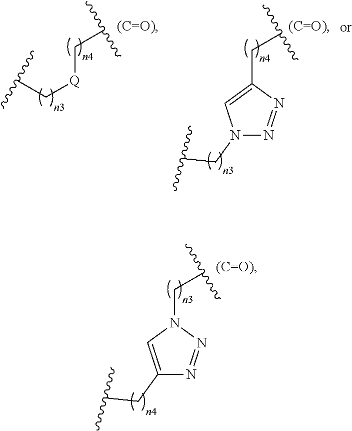

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

Accordingly, provided herein is a conjugate of Formula (I)

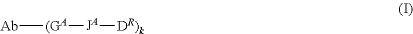

or a pharmaceutically acceptable salt thereof, wherein
Ab is an antibody that binds human B7H3, wherein the antibody comprises a VH comprising an amino acid sequence of SEQ ID NO: 7, and a VL comprising an amino acid sequence of SEQ ID NO: 8,
k is an integer from about 1 to about 10,
each G^{A} is independently a conjugation group conjugated to the antibody,
each D^{R} is independently a drug comprising a nitrogen atom N^{R},
each J^{A} is independently a group of Formula (IA)

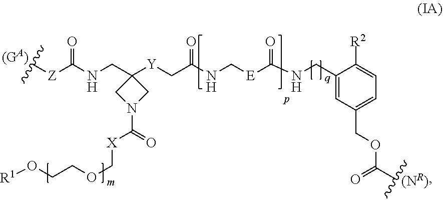

E is (CH₂)ₙ₁, wherein n1 is 0, 1, 2 or 3,
R¹ is C₁₋₄ alkyl or H,
R² is

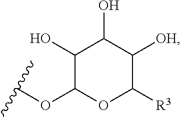

wherein R³ is CO₂H or CH₂OH,
X is (CH₂)ₙ₂,

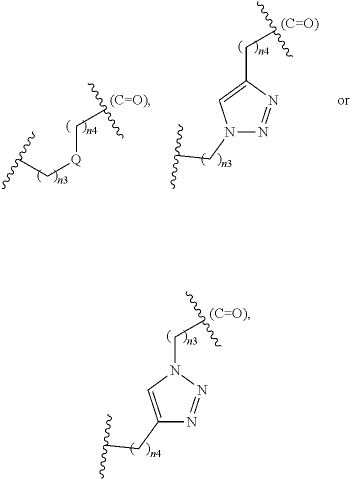

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

Accordingly, provided herein is a conjugate of Formula (I)

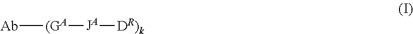

or a pharmaceutically acceptable salt thereof, wherein
Ab is an antibody that binds human B7H3, wherein the antibody comprises a heavy chain sequence of SEQ ID NO: 9 and a light chain sequence of SEQ ID NO: 11,
k is an integer from about 1 to about 10,
each G^{A} is independently a conjugation group conjugated to the antibody or antigen-binding fragment thereof,
each D^{R} is independently a drug comprising a nitrogen atom N^{R},
each J^{A} is independently a group of Formula (IA)

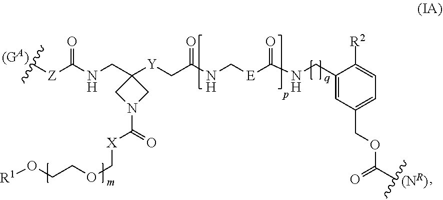

E is (CH₂)ₙ₁, wherein n1 is 0, 1, 2 or 3,
R¹ is C₁₋₄ alkyl or H,
R² is

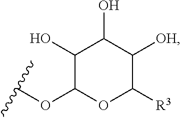

wherein R³ is CO₂H or CH₂OH,
X is (CH₂)ₙ₂,

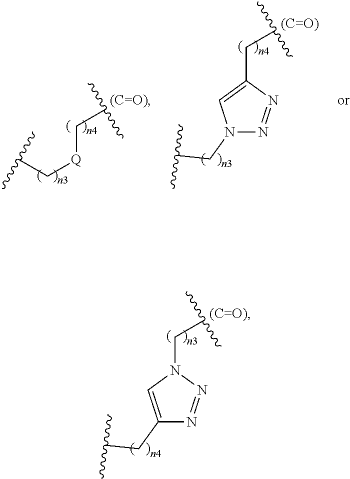

wherein n2 and n4 are 0, 1, 2 or 3, n3 is 1, 2 or 3, and Q is 0 or NR^{A}, wherein R^{A} is H, C₁₋₄ alkyl or C₃₋₄ cycloalkyl,
Y is 0 or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

In some embodiments, there is provided a conjugate of Formula (I)

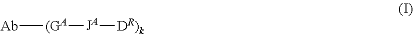

or a pharmaceutically acceptable salt thereof, wherein
Ab is an antibody that binds human B7H3, wherein the antibody comprises a heavy chain sequence of SEQ ID NO: 9 and a light chain sequence of SEQ ID NO: 11,
k is an integer from about 1 to about 10,
each G^{A} is independently a conjugation group conjugated to the antibody or antigen-binding fragment thereof,
each D^{R} is

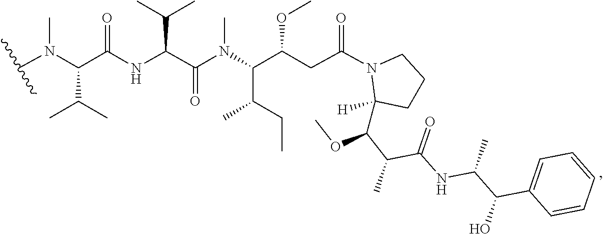 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

each J^{A} is independently a group of Formula (IA)

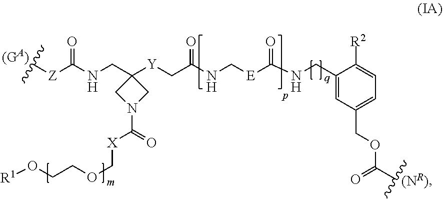

E is (CH₂)ₙ₁, wherein n1 is 0, 1, 2 or 3,
R¹ is C₁₋₄ alkyl or H,
R² is

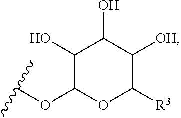

wherein R³ is CO₂H or CH₂OH,
X is (CH₂)ₙ₂,

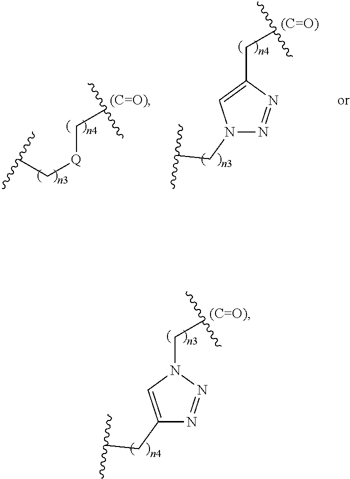

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

In other embodiments, the antibody or antigen binding fragment thereof comprises a heavy chain sequence comprising an amino acid sequence having at least 80%, at least 85%, at least 90%, at least 95%, at least 98%, at least 99%, or 100% sequence identity to the amino acid sequence of SEQ ID NO: 10 and/or a light chain sequence comprising an amino acid sequence having at least 80%, at least 85%, at least 90%, at least 95%, at least 98%, at least 99%, or 100% sequence identity to the amino acid sequence of SEQ ID NO: 11. In some embodiments, the antibody comprises a heavy chain sequence of SEQ ID NO: 10 and a light chain sequence of SEQ ID NO: 11. In other embodiments, the antibody or antigen binding fragment thereof consists of a heavy chain sequence consisting of an amino acid sequence having at least 80%, at least 85%, at least 90%, at least 95%, at least 98%, at least 99%, or 100% sequence identity to the amino acid sequence of SEQ ID NO: 10 and/or a light chain sequence consisting of an amino acid sequence having at least 80%, at least 85%, at least 90%, at least 95%, at least 98%, at least 99%, or 100% sequence identity to the amino acid sequence of SEQ ID NO: 11. In some embodiments, the antibody consists of a heavy chain sequence of SEQ ID NO: 10 and a light chain sequence of SEQ ID NO:11.

Accordingly, provided herein is a conjugate of Formula (I)

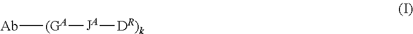

or a pharmaceutically acceptable salt thereof, wherein
Ab is an antibody that binds human B7H3, wherein the antibody comprises a heavy chain sequence of SEQ ID NO: 10 and a light chain sequence of SEQ ID NO: 11,
k is an integer from about 1 to about 10,
each G^{A} is independently a conjugation group conjugated to the antibody or antigen-binding fragment thereof,
each D^{R} is independently a drug comprising a nitrogen atom N^{R},
each J^{A} is independently a group of Formula (IA)

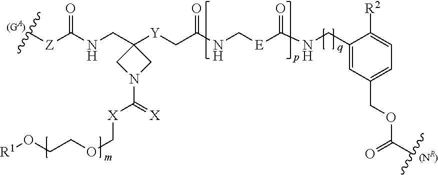

E is (CH₂)ₙ₁, wherein n1 is 0, 1, 2 or 3,
R¹ is C₁₋₄ alkyl or H,
R² is

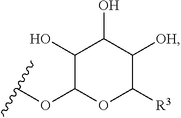

wherein R³ is CO₂H or CH₂OH,
X is (CH₂)ₙ₂,

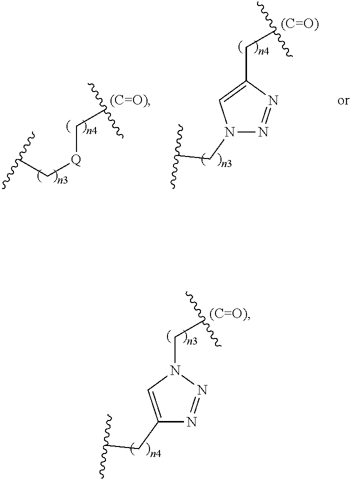

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

In some embodiments, there is provided a conjugate of Formula (I)

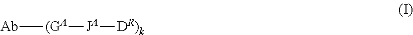

or a pharmaceutically acceptable salt thereof, wherein
Ab is an antibody that binds human B7H3, wherein the antibody comprises a heavy chain sequence of SEQ ID NO: 10 and a light chain sequence of SEQ ID NO: 11,
k is an integer from about 1 to about 10,
each G^{A} is independently a conjugation group conjugated to the antibody or antigen-binding fragment thereof,
each D^{R} is

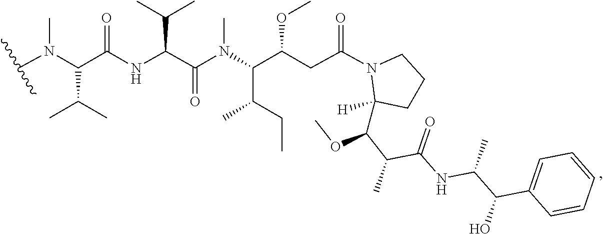

each J^{A} is independently a group of Formula (IA)

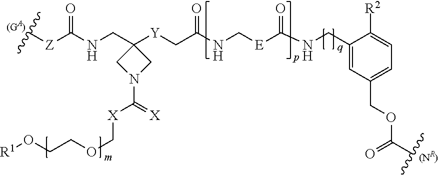

E is (CH₂)ₙ₁, wherein n1 is 0, 1, 2 or 3,
R¹ is C₁₋₄ alkyl or H,
R² is

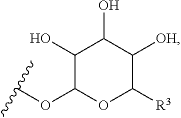

wherein R³ is CO₂H or CH₂OH,
X is (CH₂)ₙ₂,

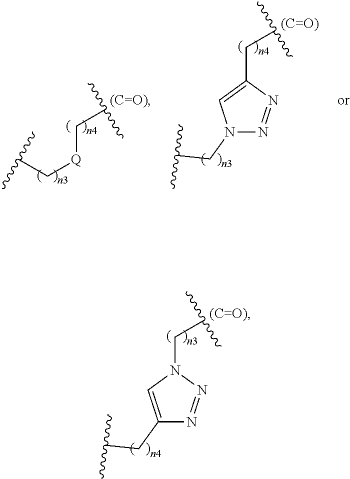

wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

In some embodiments, Ab is as described herein, in particular as set out in the section “Anti-human B7H3 antibody or antigen binding fragment thereof”.

### Drug (D^{R})

As used herein, each D^{R} is independently a drug comprising a nitrogen atom N^{R}. In the conjugate of Formula (I) or a pharmaceutically acceptable salt thereof as described herein, nitrogen atom N^{R} is covalently attached to J^{A}.

A conjugate of Formula (I) or a pharmaceutically acceptable salt thereof as described herein, may undergo cleavage to release the drug in its free drug form. D^{R} may be represented as N(R^{m})(Rⁿ), wherein N(R^{m})(Rⁿ) is collectively the drug and the nitrogen atom shown is N^{R}. In this representation, the free drug form is HN(R^{m})(Rⁿ).

In some embodiments, the nitrogen atom N^{R} may be the nitrogen atom of a secondary of tertiary carbamate. In other words, the nitrogen atom N^{R} may, in the free drug form, be the nitrogen atom of a primary or secondary amine.

In some embodiments, the nitrogen atom N^{R} in the nitrogen atom of an aliphatic secondary of tertiary carbamate. In other words, the nitrogen atom N^{R}, in the free drug form, is the nitrogen atom of an aliphatic primary or secondary amine. In some embodiments, the nitrogen atom N^{R} is the nitrogen atom of an aliphatic tertiary carbamate.

In some embodiments, the drug is a therapeutic agent. In further embodiments, the drug is a cytotoxic drug. In further embodiments, the drug is a DNA damage response inhibitor, antineoplastic agent and/or tubulin disrupting agent. In further embodiments, the drug is a microtubule inhibitor (MTI).

In some embodiments, the drug is a diagnostic agent or an imaging agent.

In some embodiments, D^{R} is

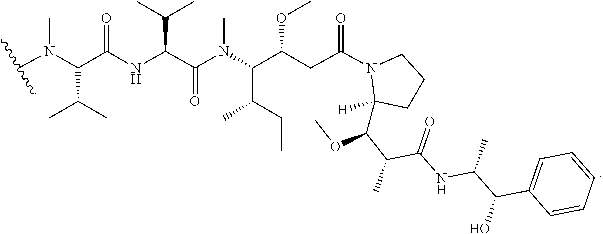

In some embodiments, the free drug form of the drug is monomethyl auristatin E (MMAE) ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide).

The released drug may, following release from the conjugate in the biological system, undergo a chemical modification, such as an enzyme-mediated chemical reaction and/or metabolism. As such, in some embodiments, the drug may be a pro-drug.

### Linker

The present specification provides a conjugate of Formula (I), or a pharmaceutically acceptable salt thereof, as described herein.

In some embodiments, k is about 1, about 2, about 3, about 4, about 5, about 6, about 7, about 8, about 9 or about 10. In further embodiments, k is an integer from about 2 to about 10. In further embodiments, k is an integer from about 2 to about 6. In further embodiments, k is an integer from about 2 to about 4. In further embodiments, k is about 4. In further embodiments k is about 2. In embodiments, k is 1, 2, 3, 4, 5, 6, 7, 8, 9 or 10. In further embodiments, k is an integer from 2 to 10. In further embodiments, k is an integer from 2 to 6. In further embodiments, k is an integer from 2 to 4. In further embodiments, k is 4. In further embodiments k is 2. In some embodiments, k is from 1 to 10. In embodiments, k is from 2 to 6. In embodiments, k is from 2 to 4. In embodiments, k is from 3 to 5.

In some embodiments, G^{A} is selected from

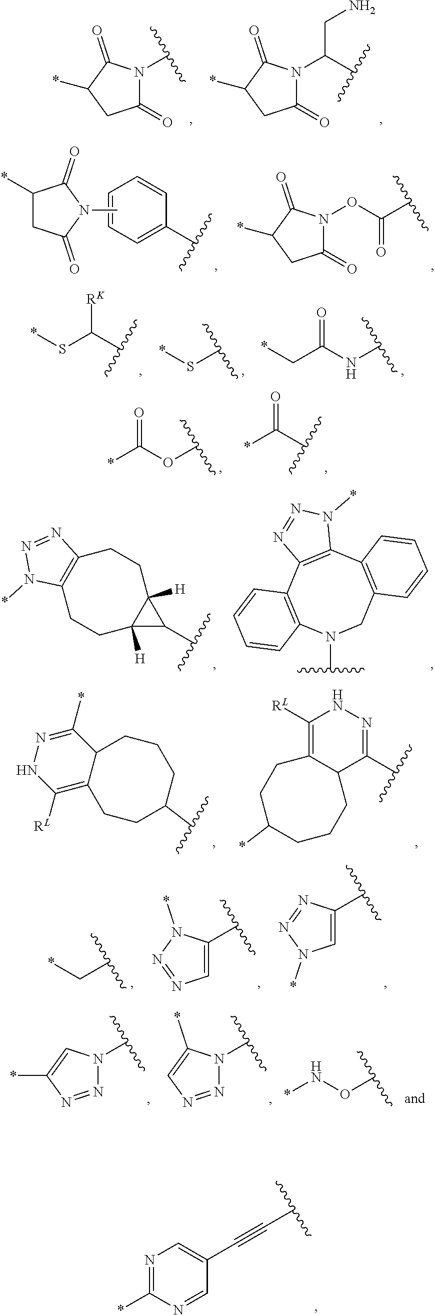

wherein R^{K} is H or CH₃, R^{L} Is C₁₋₆ alkyl, and

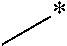 [SMILES: *C]

indicates the point of attachment to the antibody or antigen-binding fragment thereof.

In some embodiments, G^{A} is selected from

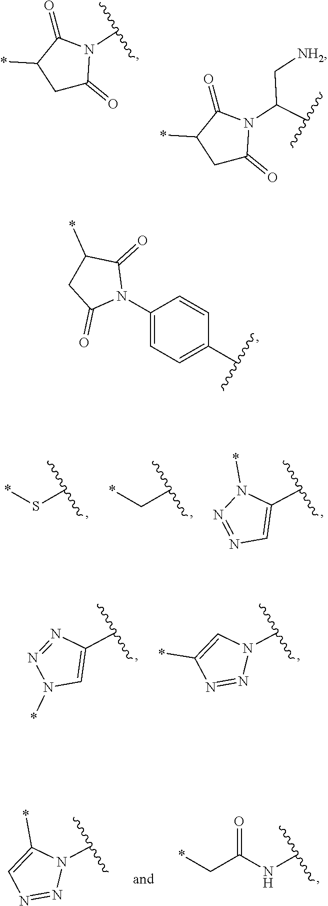

In some embodiments, G^{A} is

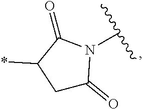 [SMILES: *C1CC(=O)N(*)C1=O]

wherein

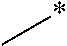 [SMILES: *C]

indicates the point of attachment to the antibody or antigen-binding fragment thereof.

In some embodiments, G^{A} is

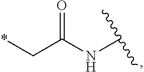 [SMILES: *CC(=O)N*]

wherein

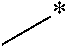 [SMILES: *C]

indicates the point of attachment to the antibody or antigen-binding fragment thereof.

In some embodiments, G^{A} is

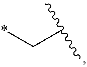 [SMILES: *C*]

wherein

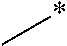 [SMILES: *C]

indicates the point of attachment to the antibody or antigen-binding fragment thereof.

In some embodiments, G^{A} is

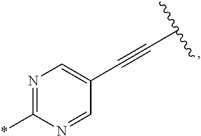 [SMILES: *C#Cc1cnc(*)nc1]

wherein

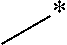 [SMILES: *C]

indicates the point of attachment to the antibody or antigen-binding fragment thereof.

In some embodiments, E is (CH₂)ₙ₁, wherein n1 is 0, 1, 2 or 3. In further embodiments, E is a covalent bond. In further embodiments, E is CH₂. In further embodiments, E is (CH₂)₂. In further embodiments, E is (CH₂)₃.

In some embodiments, R¹ is C₁₋₄ alkyl or H. In further embodiments, R¹ is C₁₋₄ alkyl. In further embodiments, R¹ is CH₃.

In some embodiments, R² is

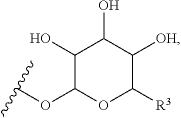

wherein R³ is CO₂H or CH₂OH. In further embodiments, R³ is CO₂H.

In some embodiments, R² is

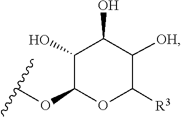

wherein R³ is CO₂H or CH₂OH. In further embodiments, R³ is CO₂H.

In some embodiments, R² is

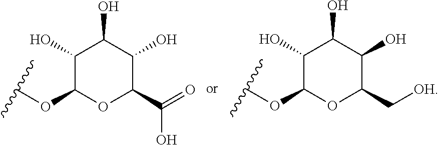 [SMILES: *O[C@@H]1O[C@H](C(=O)O)[C@@H](O)[C@H](O)[C@H]1O ; "or" ; *O[C@@H]1O[C@H](CO)[C@H](O)[C@H](O)[C@H]1O]

In some embodiments, R² is

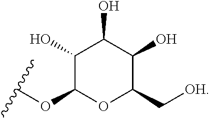 [SMILES: *O[C@@H]1O[C@H](CO)[C@H](O)[C@H](O)[C@H]1O]

In some embodiments, R² is

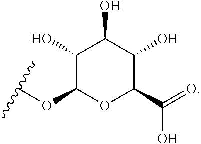 [SMILES: *O[C@@H]1O[C@H](C(=O)O)[C@@H](O)[C@H](O)[C@H]1O]

In some embodiments, X is (CH₂)ₙ₂, wherein n2 is 0, 1, 2 or 3. In further embodiments, X is a covalent bond. In further embodiments, X is CH₂. In further embodiments, X is (CH₂)₂. In further embodiments, X is (CH₂)₃.

In some embodiments, X is

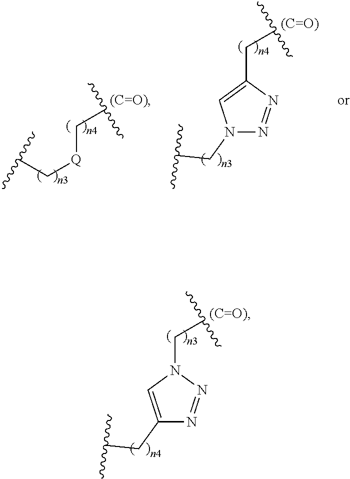

wherein n4 is 0, 1, 2 or 3, n3 is 1, 2 or 3, and Q is O or NR^{A}, and wherein R^{A} is H, C₁₋₄ alkyl or C₃₋₄ cycloalkyl. As used herein ^{(C═O)} indicates the point of attachment to C═O.

In some embodiments, Y is O.

In some embodiments, Y is NR^{B}, wherein R^{B} is H, C₁₋₄ alkyl or C₃₋₄ cycloalkyl. In further embodiments, Y is NH.

In some embodiments, Z is (CH₂)ₙ₅, wherein n5 is 1, 2, 3, 4 or 5. In further embodiments, Z is CH₂. In further embodiments, Z is (CH₂)₂. In further embodiments, Z is (CH₂)₃. In further embodiments, Z is (CH₂)₄. In further embodiments, Z is (CH₂)₅.

In some embodiments, m is 2, 3, 4, 5, 6, 7, 8, 9, 10, 11, 12, 13, 14, 15, 16 or 17. In further embodiments, m is an integer from 6 to 16. In further embodiments, m is an integer from 7 to 15. In further embodiments, m is an integer from 8 to 14. In further embodiments, m is an integer from 9 to 13. In some embodiments, m is 9, 10, 11, 12 or 13. In further embodiments, m is an integer from 10 to 12. In further embodiments, m is 11.

In some embodiments, p is 1. In other embodiments, p is 0.

In some embodiments, q is 1. In other embodiments, q is 0.

In some embodiments, each J^{A} is a group of Formula (IB)

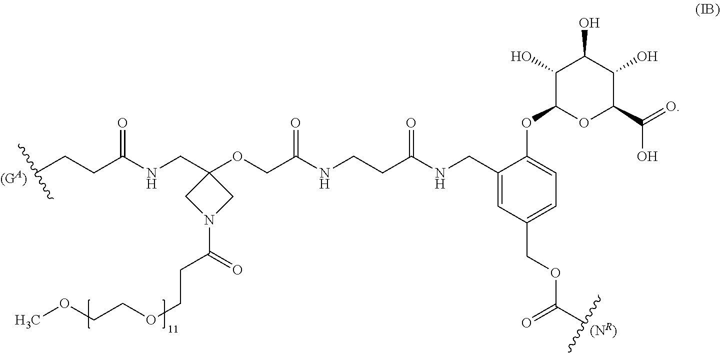

In some embodiments, each J^{A} is a group of Formula (IB1)

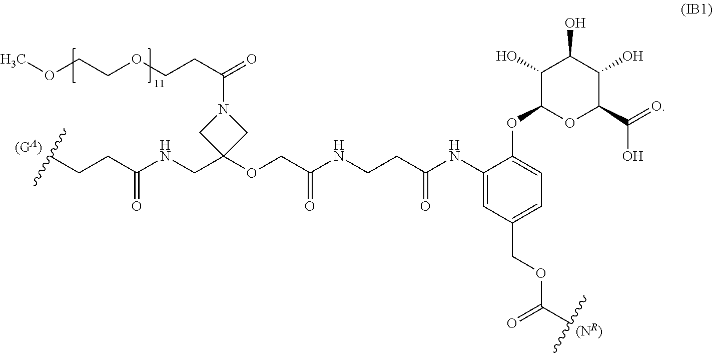

In some embodiments, each J^{A} is a group of Formula (IB2)

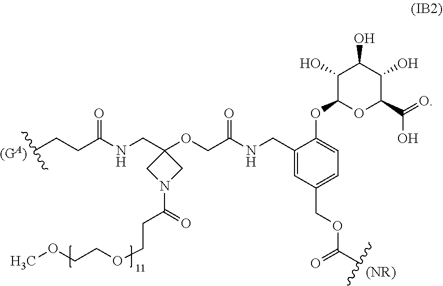

In some embodiments, each J^{A} is a group of Formula (IB3)

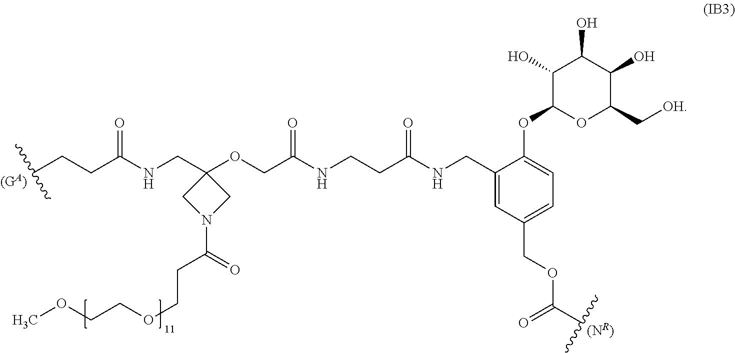

In some embodiments, each J^{A} is a group of Formula (IB4)

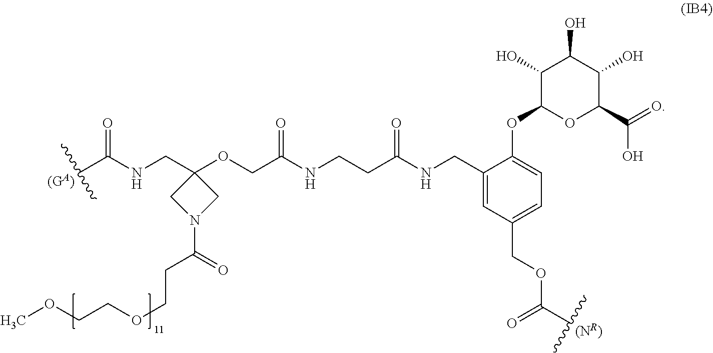

In some embodiments, each G^{A}-J^{A}-D^{R} is a group of Formula (IC)

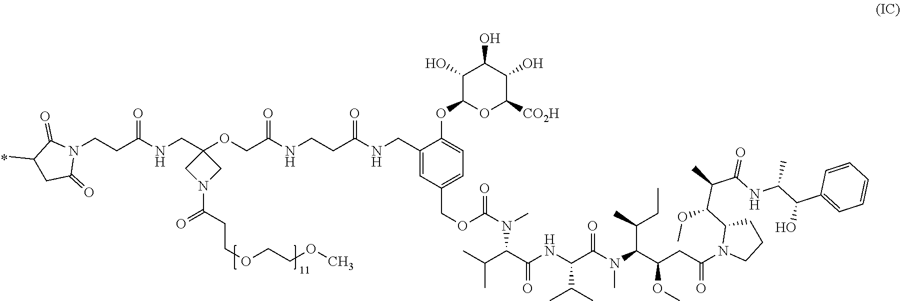

wherein

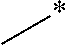 [SMILES: *C]

indicates the point of attachment to the antibody or antigen-binding fragment thereof.

In some embodiments, each G^{A}-J^{A}-D^{R} is

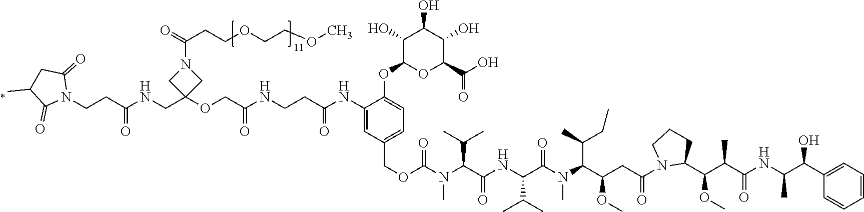

wherein

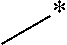 [SMILES: *C]

indicates the point of attachment to the antibody or antigen-binding fragment thereof.

In some embodiments, each G^{A}-J^{A}-D^{R} is

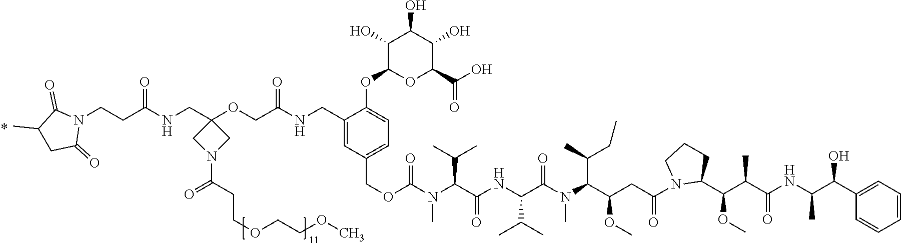

wherein

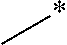 [SMILES: *C]

indicates the point of attachment to the antibody or antigen-binding fragment thereof.

In some embodiments, each G^{A}-J^{A}-D^{R} is

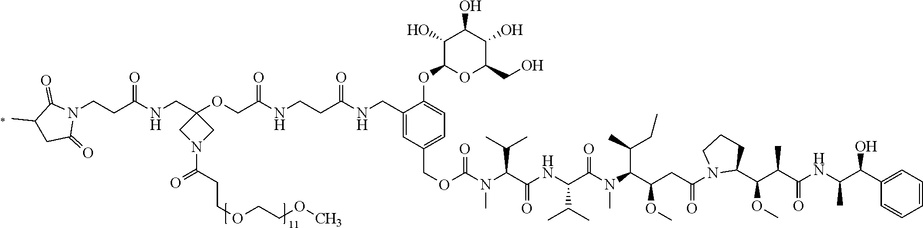

wherein

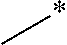 [SMILES: *C]

indicates the point of attachment to the antibody or antigen-binding fragment thereof.

In some embodiments, each G^{A}-J^{A}-D^{R} is

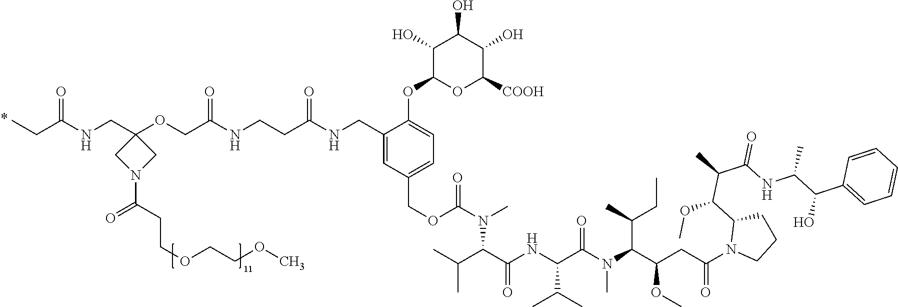

wherein

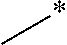 [SMILES: *C]

indicates the point of attachment to the antibody or antigen-binding fragment thereof.

The present specification provides a conjugate of Formula (I)

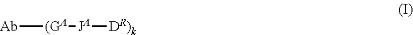

or a pharmaceutically acceptable salt thereof, wherein
Ab is an antibody that binds human B7H3 comprising a heavy chain sequence of SEQ ID NO: 9 and a light chain sequence of SEQ ID NO: 11,
k is about 2, and
each G^{A}J^{A}-D^{R} is a group of Formula (IC)

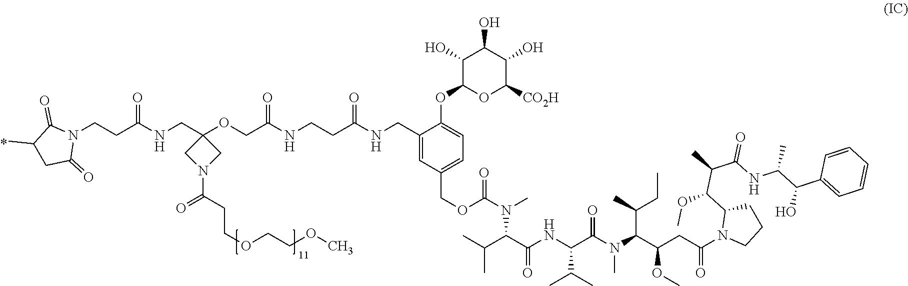

wherein

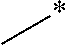 [SMILES: *C]

indicates the point of attachment to the antibody. In some embodiments, each G^{A}-J^{A}-D^{R} group is conjugated to a cysteine at position 174 of SEQ ID NO: 9.

The present specification provides a conjugate of Formula (I)

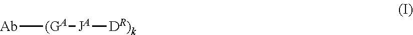

or a pharmaceutically acceptable salt thereof, wherein
Ab is an antibody that binds human B7H3 consisting of a heavy chain sequence of SEQ ID NO: 9 and a light chain sequence of SEQ ID NO: 11,
k is about 2, and
each G^{A}-J^{A}-D^{R} is a group of Formula (IC)

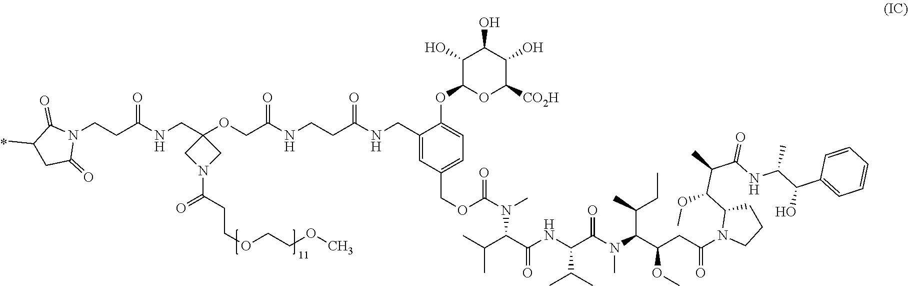

wherein

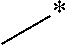 [SMILES: *C]

indicates the point of attachment to the antibody. In some embodiments, each G^{A}-J^{A}-D^{R} group is conjugated to a cysteine at position 174 of SEQ ID NO: 9.

The present specification also provides a conjugate of Formula (I)

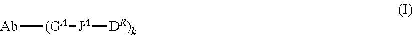

or a pharmaceutically acceptable salt thereof, wherein
Ab is an antibody that binds human B7H3 comprising a heavy chain sequence of SEQ ID NO: 10 and a light chain sequence of SEQ ID NO: 11,
k is about 4, and
each G^{A}J^{A}-D^{R} is a group of Formula (IC)

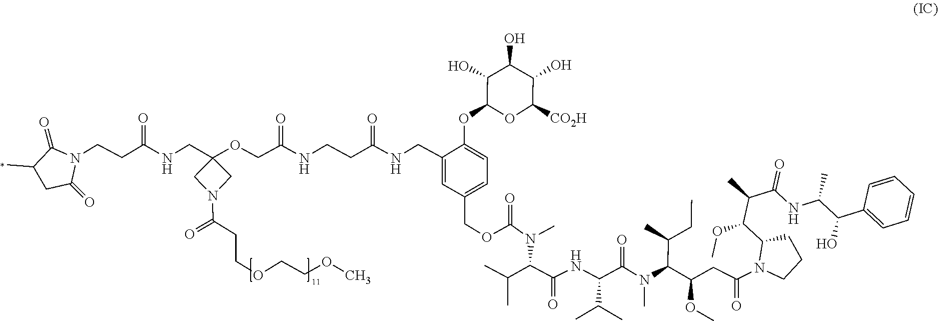

wherein

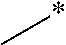 [SMILES: *C]

indicates the point of attachment to the antibody.

The present specification also provides a conjugate of Formula (I)

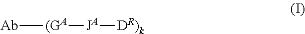

or a pharmaceutically acceptable salt thereof, wherein
Ab is an antibody that binds human B7H3 consisting of a heavy chain sequence of SEQ ID NO: 10 and a light chain sequence of SEQ ID NO: 11,
k is about 4, and
each G^{A}-J^{A}-D^{R} is a group of Formula (IC)

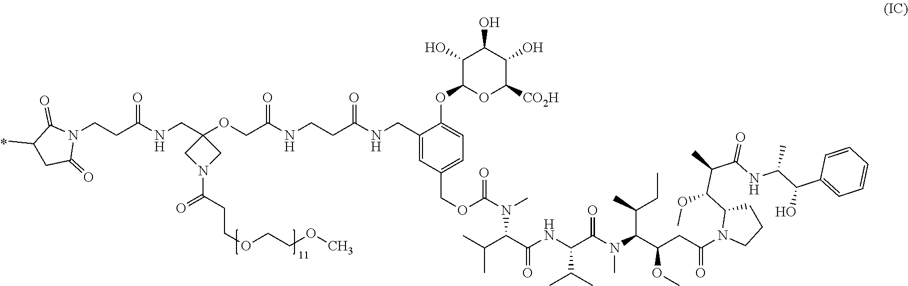

wherein

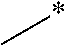 [SMILES: *C]

indicates the point of attachment to the antibody.

The present specification also provides a conjugate of Formula (I)

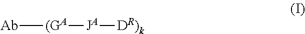

or a pharmaceutically acceptable salt thereof, wherein
Ab is an antibody that binds human B7H3 comprising a VH comprising an amino acid sequence of SEQ ID NO: 7 and a VL comprising an amino acid sequence of SEQ ID NO: 8,
k is from about 2 to about 4, and
each G^{A}-J^{A}-D^{R} is a group of Formula (IC)

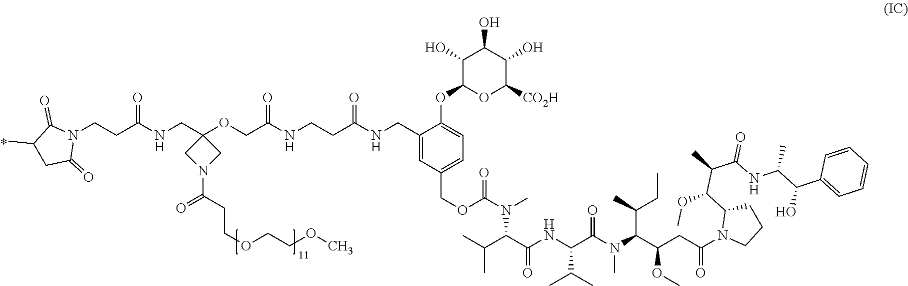

wherein

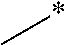 [SMILES: *C]

indicates the point of attachment to the antibody.

### Salts and Solvates

The compounds of the present disclosure (e.g. ADCs, linker-drug structure, and intermediates thereof, as described herein) may exist in salt form or in non-salt form (i.e., as a free base), and the present disclosure covers both salt forms and non-salt forms. The compounds may form acid addition salts or base addition salts.

In general, an acid addition salt can be prepared using various inorganic or organic acids. Examples of inorganic acids include hydrochloric acid, hydrobromic acid, sulphuric acid and phosphoric acid. Examples of organic acids include trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid. Such salts can typically be formed by, for example, mixing the compound with an acid (e.g., a stoichiometric amount of an acid) using various methods known in the art. This mixing may occur in water, an organic solvent (e.g., ether, ethyl acetate, ethanol, methanol, isopropanol, or acetonitrile), or an aqueous/organic mixture.

In general, a base addition salt can be prepared using various inorganic or organic bases. The skilled person will be aware of the general principles and techniques of preparing pharmaceutical salts, such as those described in, for example, Berge, S., et al., “Pharmaceutical Salts,” J. Pharm. Sci. 66, 1 (1977). A base addition salt may for example be an alkali metal salt (such as a sodium, potassium, or lithium salt) or an alkaline earth metal salt (such as a calcium salt), which may be formed using an alkali metal or alkaline earth metal hydroxide or alkoxide (e.g., an ethoxide or methoxide). A base addition salt may also be formed using a suitably basic organic amine (e.g., a choline or meglumine salt). Examples of pharmaceutically acceptable salts are also described in “Handbook of Pharmaceutical Salts: Properties, Selection, and Use” by Stahl and Wermuth (Wiley-VCH, Weinheim, Germany, 2002).

It will be understood that if a compound described herein contains two or more basic moieties, the stoichiometry of salt formation may include 1, 2 or more equivalents of acid. Such salts would contain 1, 2 or more acid counterions, for example, a dihydrochloride salt.

A further suitable pharmaceutically acceptable salt of a conjugate of Formula (I) is, for example, a salt formed within a patient's body after administration of a conjugate of Formula (I) to the patient.

Stoichiometric and non-stoichiometric forms of a pharmaceutically acceptable salt of a compound described herein are included within the scope of the specification, including sub-stoichiometric salts, for example where a counterion contains more than one acidic proton.

It is also to be understood that certain compounds disclosed herein, and pharmaceutically salts thereof, can exist in solvated as well as unsolvated forms such as, for example, hydrated and anhydrous forms. It is to be understood that the compounds herein encompass all such solvated forms. For the sake of clarity, this includes both solvated (e.g., hydrated) forms of the free form of the compound, as well as solvated (e.g., hydrated) forms of the salt of the compound.

### Isotopes

Any formula given herein is intended to represent unlabeled forms as well as isotopically labeled forms for the compounds disclosed herein (e.g. ADCs, linker-drug structure, and intermediates thereof, as described herein). Isotopically labeled compounds have structures depicted by the formulas given herein except that one or more atoms are replaced by an atom of the same element but with differing mass number. Examples of isotopes that can be incorporated into compounds of the specification and pharmaceutically acceptable salts thereof include isotopes of hydrogen, carbon, nitrogen, oxygen, phosphorous, sulfur, fluorine, chlorine, and iodine, such as ²H, ³H, ¹¹C, ¹³C, ¹⁴C, ¹⁵N, ¹⁷O, ¹⁸O ³¹P, ³²P ³⁵S ¹⁸F, ³⁶Cl, ¹²³I, and ¹²⁵I. Isotopically labeled compounds disclosed herein can generally be prepared by conventional techniques known to those skilled in the art or by processes analogous to those described in the accompanying Examples using appropriate isotopically labeled reagents in place of the non-labeled reagents previously employed.

Compounds of the present specification (e.g. ADCs, linker-drug structure, and intermediates thereof, as described herein) and pharmaceutically acceptable salts of said compounds that contain the aforementioned isotopes and/or other isotopes of other atoms are within the scope of the present specification. Isotopically-labeled compounds of the present specification, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly used for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be used in some circumstances. Isotopically labeled compounds described herein can be prepared by carrying out the procedures disclosed in the Examples below, by substituting a readily available isotopically labeled reagent for a non-isotopically labeled reagent.

### Isomers

The compounds disclosed herein (e.g. ADCs, linker-drug structure, and intermediates thereof, as described herein) may have different isomeric forms. The language “optical isomer”, “stereoisomer”, “enantiomer” or “diastereoisomer” refers to any of the various stereoisomeric configurations which may exist for a given compound disclosed herein. It is understood that a substituent may be attached at a chiral center of a carbon atom and, therefore, the disclosed compounds include enantiomers, diastereomers and racemates. The term “enantiomer” includes pairs of stereoisomers that are non-superimposable mirror images of each other. A 1:1 mixture of a pair of enantiomers is a racemic mixture. The (+/−) term is used to designate a racemic mixture where appropriate. The terms “diastereomers” or “diastereoisomers” include stereoisomers that have at least two asymmetric atoms, but which are not mirror images of each other. The absolute stereochemistry is specified according to the Cahn-Ingold-Prelog R—S system. When a compound is a pure enantiomer, the stereochemistry at each chiral center may be specified by either R or S.

The compounds disclosed herein may contain one or more chiral centers. Accordingly, if desired, such compounds can be prepared or isolated as pure stereoisomers, i.e. as individual enantiomers, diastereoisomers, or as a stereoisomerically enriched mixture. All such stereoisomer (and enriched) mixtures are included within the scope of the embodiments, unless otherwise stated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

It is understood that one of skill in the art could determine optical rotation and/or absolute stereochemistry. It is understood that such a disclosure includes other stereoisomeric forms of the same compound, as well as stereoisomeric mixtures. Likewise, it is understood that a compound or salt of a compound disclosed herein may exist in tautomeric forms other than that shown in the formula and these are also included within the scope of the present specification. “Tautomers” are structural isomers that exist in equilibrium resulting from the migration of a hydrogen atom. Certain compounds disclosed herein contain one or more asymmetric centers or axes and may thus give rise to enantiomers, diastereomers or other stereoisomeric forms that may be defined, in terms of absolute stereochemistry, as (R)- or (S)-. The present disclosure is meant to include all such possible isomers, including racemic mixtures, optically pure forms and intermediate mixtures. Optically active (R)- and (S)-isomers may be prepared using chiral synthons or chiral reagents, or resolved using conventional techniques well known in the art, such as chiral HPLC. The scope of the present specification includes mixtures of stereoisomers as well as purified enantiomers or enantiomerically/diastereomerically enriched mixtures.

Unless stereochemistry is explicitly indicated in a chemical structure or chemical name, the chemical structure or chemical name is intended to embrace all possible stereoisomers, diastereoisomers, conformers, rotamers and tautomers of the compound depicted. For example, a compound containing a chiral carbon atom is intended to embrace both the (R) enantiomer and the (S) enantiomer, as well as mixtures of the enantiomers, including racemic mixtures; and a compound containing two chiral carbons is intended to embrace all enantiomers and diastereoisomers including (R,R), (S,S), (R,S) and (S,R).

### Preparation of Conjugates

The present disclosure provides a method of making a conjugate as described herein. The present disclosure also provides intermediates for the preparation of a conjugate as described herein.

### Preparation of Conjugation Group Linker-Drug

The present specification provides a compound of Formula (II)

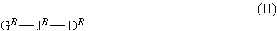

or a salt thereof, wherein G^{B} is a conjugation group for conjugation to an antibody or antigen-binding fragment thereof,
D^{R} is a drug comprising a nitrogen atom N^{R},
J^{B} is a group of Formula (IIA)

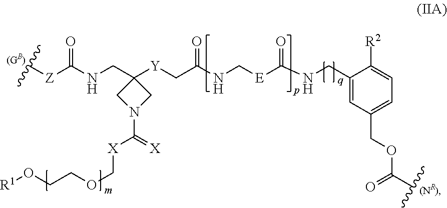

wherein E, R¹, R², X, Y, Z, m, p and q are as defined in any of the embodiments of a conjugate of Formula (I) disclosed herein, (G^{B}) indicates the point of attachment to G^{B}, and (N^{R}) indicates the point of attachment to the nitrogen atom N^{R}.

In some embodiments, G^{B} is selected from

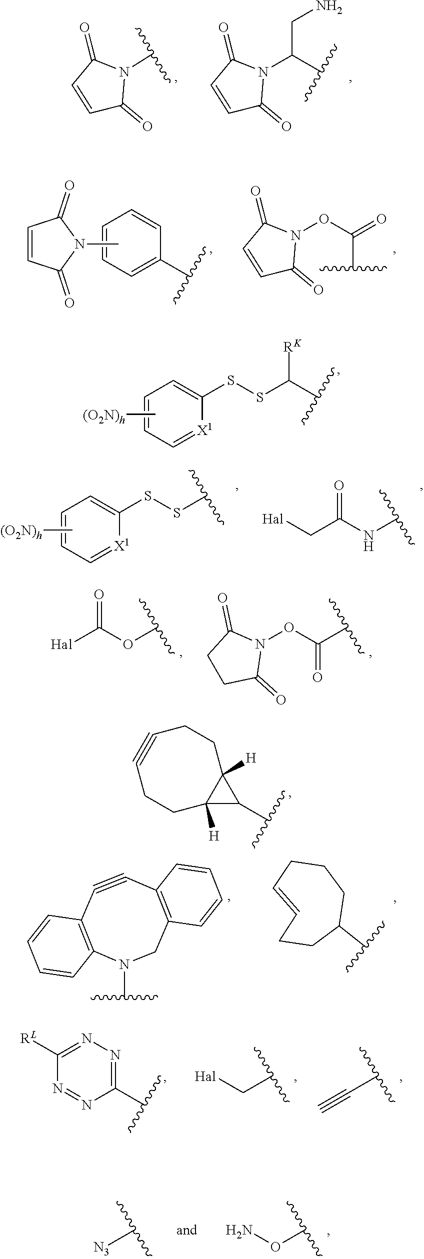

wherein X¹ is CH or N, h is 0 or 1, Hal is Cl, Br or I, R^{K} is H or CH₃, and R^{L} is C₁₋₆ alkyl.

In some embodiments, G^{B} is selected from

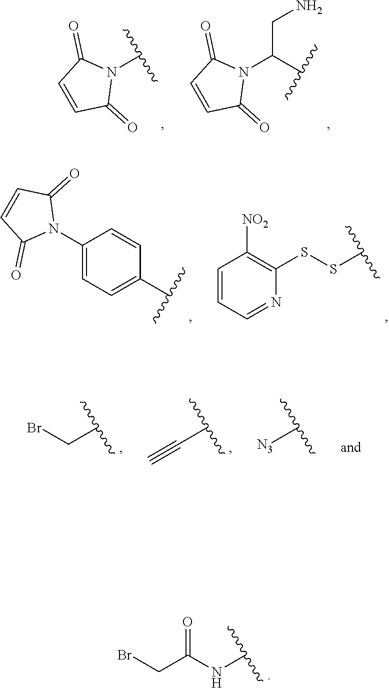

In some embodiments, G^{B} is

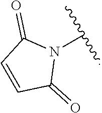

In some embodiments, G^{B} is

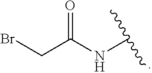 [SMILES: *NC(=O)CBr]

In some embodiments, G^{B} is

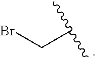 [SMILES: *CBr]

In some embodiments. G^{B} is

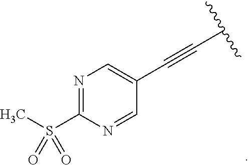 [SMILES: *C#Cc1cnc(S(C)(=O)=O)nc1]

In some embodiments, J^{B} is a group of Formula (IIB)

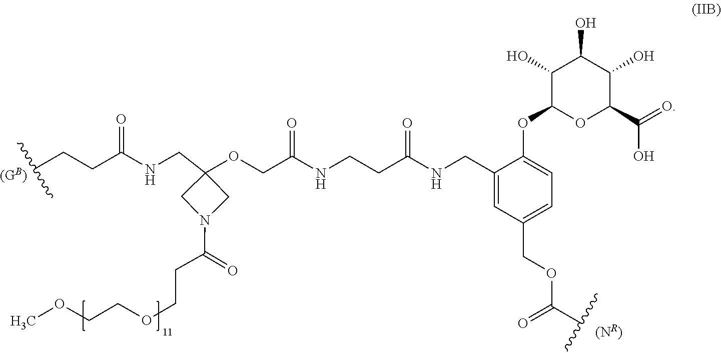

In some embodiments, J^{B} is a group of Formula (IIB1)

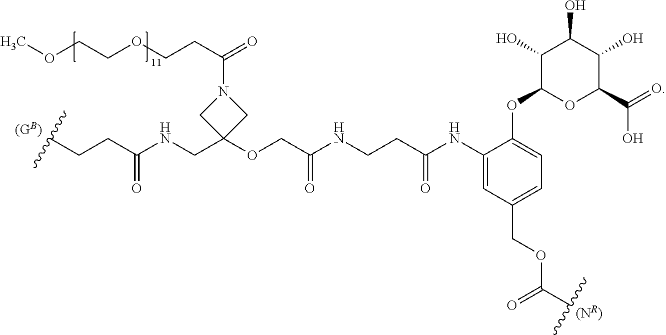

In some embodiments, J^{B} is a group of Formula (IIB2)

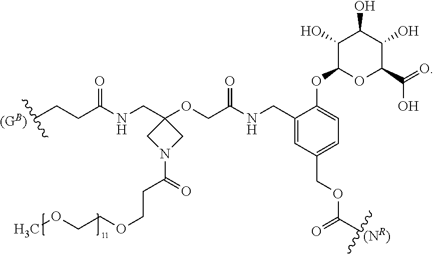

In some embodiments, J^{B} is a group of Formula (IIB3)

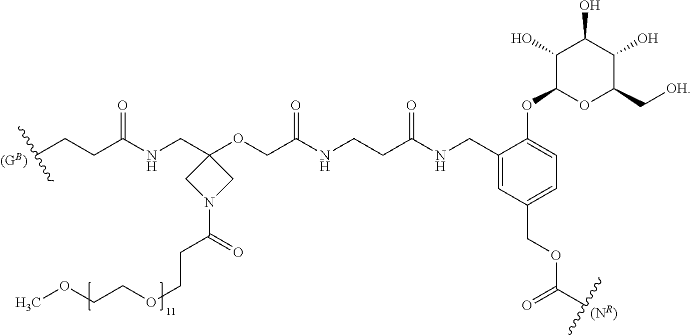

In some embodiments J^{B} is a group of Formula (IB4)

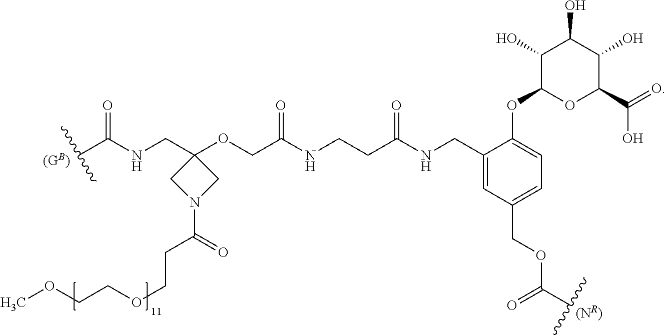

In some embodiments, D^{R} is as described herein.

In some embodiments, there is provided a compound of Formula (II) or a salt thereof, that is

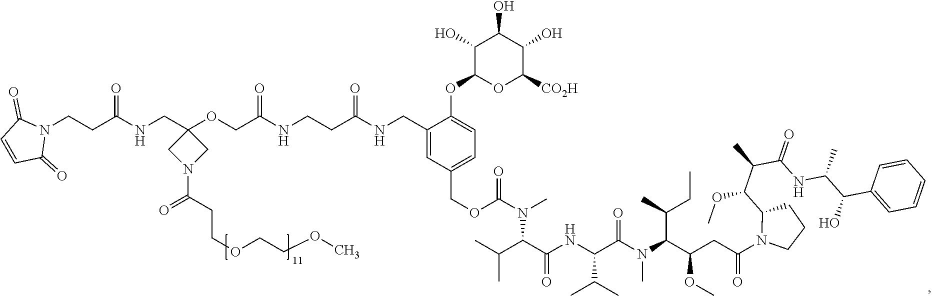

(2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((3-(2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetamido)propanamido)methyl)phenoxy)-3,4,5-trihydroxytetrahydro-2H-pyran-2-carboxylic acid, or a salt thereof.

In some embodiments, there is provided a compound of Formula (II) or a salt thereof, that is

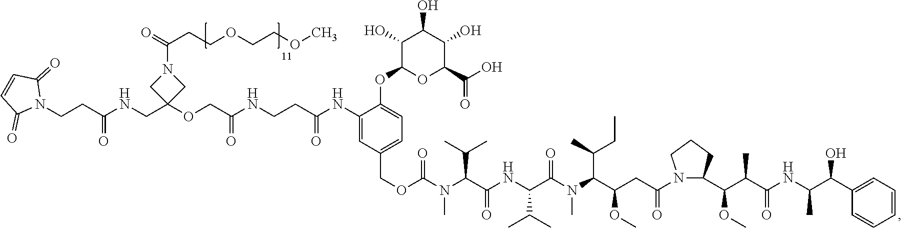

(2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(3-(2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetamido)propanamido)phenoxy)-3,4,5-trihydroxytetrahydro-2H-pyran-2-carboxylic acid, or a salt thereof.

In some embodiments, there is provided a compound of Formula (II) or a salt thereof, that is

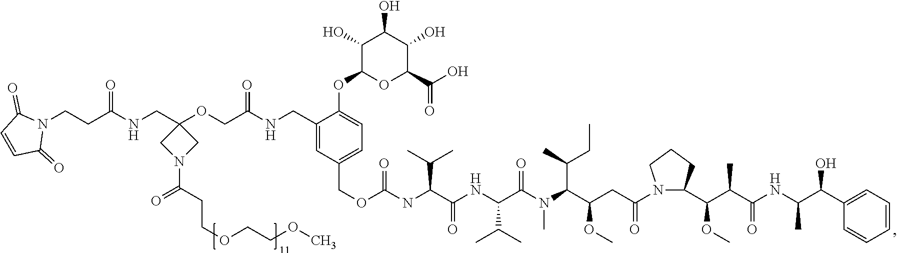

(2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetamido)methyl)phenoxy)-3,4,5-trihydroxytetrahydro-2H-pyran-2-carboxylic acid, or a salt thereof.

In some embodiments, there is provided a compound of Formula (II) or a salt thereof, that is

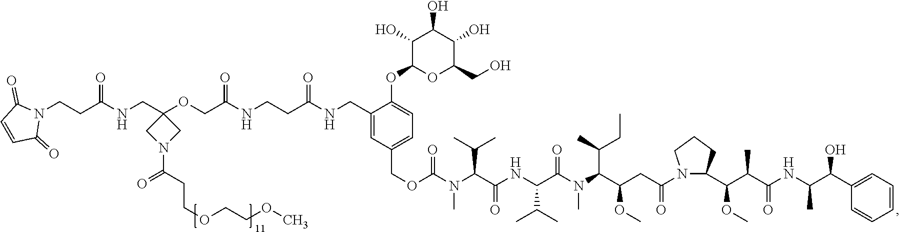

3-((3-(2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetamido)propanamido)methyl)-4-(((2S,3R,4S,5R,6R)-3,4,5-trihydroxy-6-(hydroxymethyl)tetrahydro-2H-pyran-2-yl)oxy)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate, or a salt thereof.

In some embodiments, there is provided a compound of Formula (II) or a salt thereof, that is

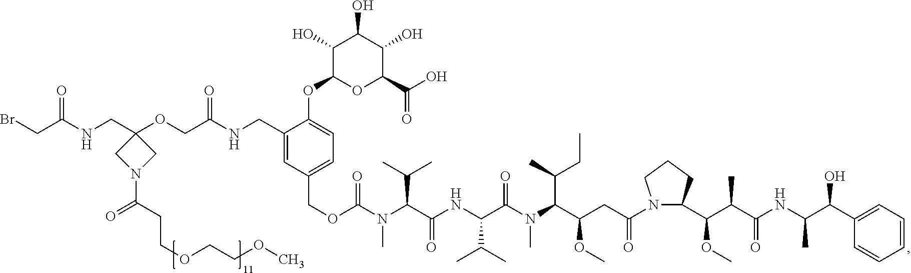

(2S,3S,4S,5R,6S)-6-(2-((3-(2-((3-((2-bromoacetamido)methyl)-1-(2,5,8,11,14,17,20,23,26,29,32,35-dodecaoxaoctatriacontan-38-oyl)azetidin-3-yl)oxy)acetamido)propanamido)methyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid, or a salt thereof.

The present specification also provides a compound of Formula (III)

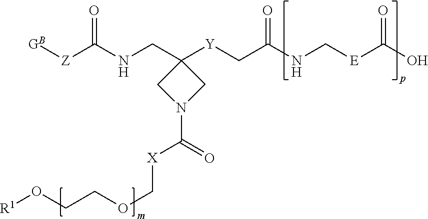

or a salt thereof, wherein G^{B} is a conjugation group for conjugation to an antibody or antigen-binding fragment thereof as described herein, and E, R¹, X, Y, Z, m and p are as defined in any embodiment of a conjugate of Formula (I).

In some embodiments, there is provided a compound of Formula (III), or a salt thereof, that is 3-(2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetamido)propanoic acid, or a salt thereof.

In some embodiments, there is provided a compound of Formula (III), or a salt thereof, that is 2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetic acid, or a salt thereof.

Also provided herein is a compound of Formula (IV)

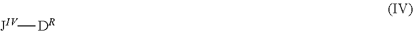

or a salt thereof, wherein D^{R} is a drug comprising a nitrogen atom N^{R},
J^{IV} is a group of Formula (IVA)

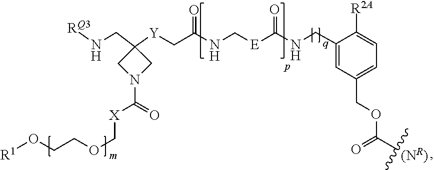

wherein E, R¹, X, Y, m, p and q are as defined in any of the embodiments of a conjugate of Formula (I) disclosed herein, R^{2A}

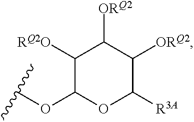

wherein R^{3A} is CO₂R^{Q1} or CH₂OR^{Q2}, R^{Q1} is H or R¹, each R^{Q2} is independently is H or R^{P2}, and R^{Q3} is H or R^{P3}, wherein R^{P1} is a carboxylic acid protecting group, each R^{P2} is independently an alcohol protecting group and R^{P3} is an amine protecting group, and (N^{R}) indicates the point of attachment to the nitrogen atom N^{R}.

In some embodiments, there is provided a compound of Formula (IV) or a salt thereof, wherein R^{2A} is

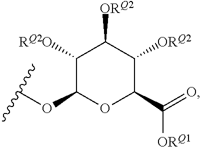

In further embodiments, R^{2A} is

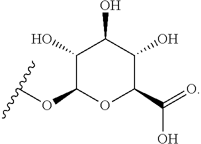 [SMILES: *O[C@@H]1O[C@H](C(=O)O)[C@@H](O)[C@H](O)[C@H]1O]

In some embodiments, there is provided a compound of Formula (IV) or a salt thereof, wherein R^{2A} is

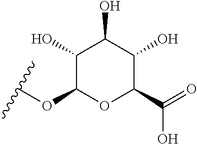 [SMILES: *O[C@@H]1O[C@H](C(=O)O)[C@@H](O)[C@H](O)[C@H]1O]

### and R^{Q3} is H.

In some embodiments, there is provided a compound of Formula (IV) or a salt thereof, wherein R^{2A} is

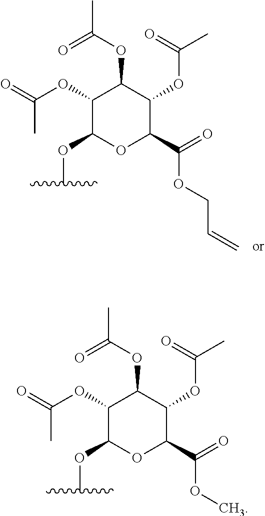 [SMILES: *O[C@@H]1O[C@H](C(=O)OCC=C)[C@@H](OC(C)=O)[C@H](OC(C)=O)[C@H]1OC(C)=O ; *O[C@@H]1O[C@H](C(=O)OC)[C@@H](OC(C)=O)[C@H](OC(C)=O)[C@H]1OC(C)=O]

In further embodiments, R^{Q3} is C(O)OCH₂CH═CH₂.

In some embodiments, there is provided a compound of Formula (IV) or a salt thereof, that is

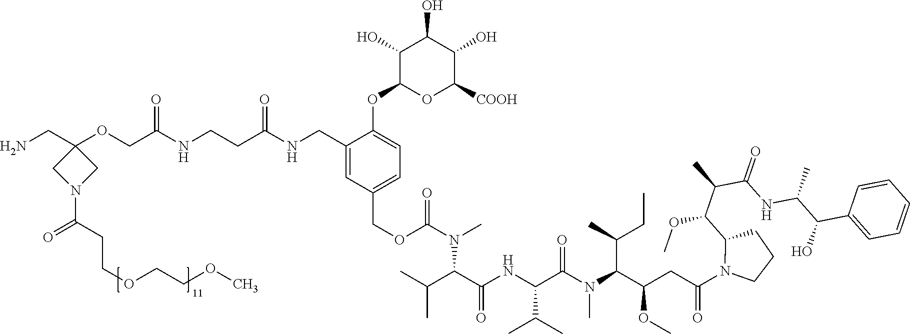

(2S,3S,4S,5R,6S)-6-(2-((3-(2-((3-(aminomethyl)-1-(2,5,8,11,14,17,20,23,26,29,32,35-dodecaoxaoctatriacontan-38-oyl)azetidin-3-yl)oxy)acetamido)propanamido)methyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid, or a salt thereof.

In some embodiments, there is provided a compound of Formula (IV), or a salt thereof, that is

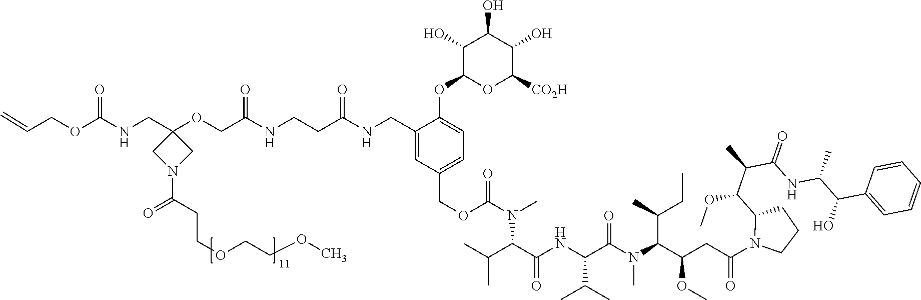

(2S,3S,4S,5R,6S)-6-[2-[[3-[[2-[3-[(allyloxycarbonylamino)methyl]-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenoxy]-3,4,5-trihydroxy-tetrahydropyran-2-carboxylic acid, or a salt thereof.

In some embodiments, there is provided a compound of Formula (IV), or a salt thereof, that is

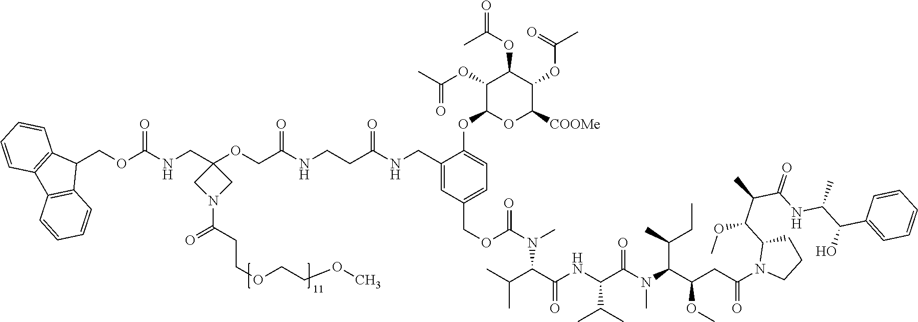

(2S,3R,4S,5S,6S)-2-(2-((3-(2-((3-(((((9H-fluoren-9-yl)methoxy)carbonyl)amino)methyl)-1-(2,5,8,11,14,17,20,23,26,29,32,35-dodecaoxaoctatriacontan-38-oyl)azetidin-3-yl)oxy)acetamido)propanamido)methyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate, or a salt thereof.

In some embodiments, there is provided a compound of Formula (IV), or a salt thereof, that is

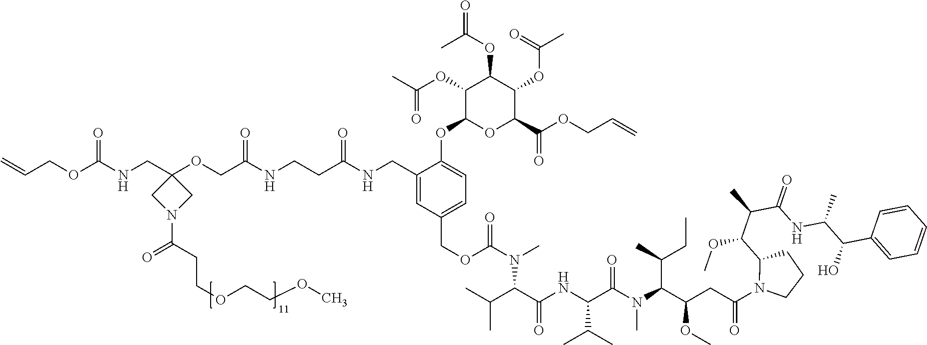

allyl (2S,3S,4S,5R,6S)-3,4,5-triacetoxy-6-[2-[[3-[[2-[3-[(allyloxycarbonylamino)methyl]-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-[[[(1S)-1-[[(1S)-1-[[(1S,2R)-4-[(2S)-2-[(1R,2R)-3-[1[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenoxy]tetrahydropyran-2-carboxylate, or a salt thereof.

The present specification also provides a compound of Formula (V)

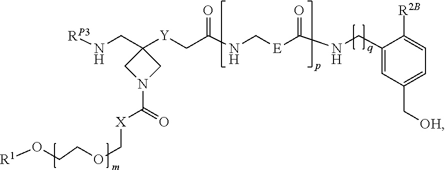

or a salt thereof, wherein E, R¹, X, Y, m, p and q are as defined in any of the embodiments of a conjugate of Formula (I) disclosed herein, R^{2B} is

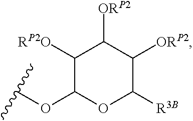

wherein R^{3B} is CO₂R^{P1} or CH₂OR^{P2}, R^{P1} is a carboxylic acid protecting group, each R^{P2} is independently an alcohol protecting group and R^{P3} is an amine protecting group.

In some embodiments, there is provided a compound of Formula (V), or a salt thereof, that is

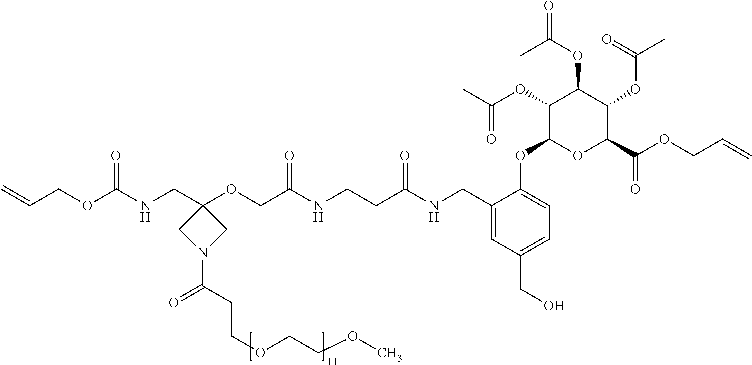

allyl (2S,3S,4S,5R,6S)-3,4,5-triacetoxy-6-[2-[[3-[[2-[3-[(allyloxycarbonylamino)methyl]-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-(hydroxymethyl)phenoxy]tetrahydropyran-2-carboxylate, or a salt thereof.

In some embodiments, there is provided a compound of Formula (V), or a salt thereof, that is

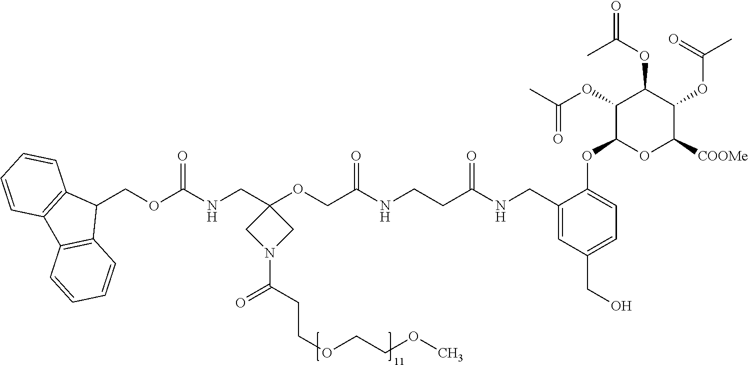

(2S,3R,4S,5S,6S)-2-(2-((3-(2-((3-(((((9H-fluoren-9-yl)methoxy)carbonyl)amino)methyl)-1-(2,5,8,11,14,17,20,23,26,29,32,35-dodecaoxaoctatriacontan-38-oyl)azetidin-3-yl)oxy)acetamido)propanamido)methyl)-4-(hydroxymethyl)phenoxy)-6-(methoxycarbonyl)tetrahydro-2H-pyran-3,4,5-triyl triacetate, or a salt thereof.

In some embodiments, there is provided a compound of Formula (IV) or (V), or a salt thereof, wherein R^{P1} is selected from benzyl, allyl and C₁₋₄ alkyl. In further embodiments, R¹ is allyl. In further embodiments, R¹ is methyl.

In some embodiments, there is provided a compound of Formula (IV) or (V), or a salt thereof, wherein each R^{P2} is independently selected from benzyl, acetyl, and CH₂CH═CH₂. In further embodiments, each R^{P2} is acetyl.

In some embodiments, there is provided a compound of Formula (IV) or (V), or a salt thereof, wherein R^{P3} is selected from C(O)OCH₂CH═CH₂, trifluoroacetyl, tert-butyloxycarbonyl (Boc), benzyloxycarbonyl (Cbz), benzyl, 2,4-dimethoxybenzyl and fluorenylmethoxycarbonyl (Fmoc). In further embodiments, R^{P3} is C(O)OCH₂CH═CH₂. In further embodiments, R^{P3} is Fmoc.

Conjugation with Antibody or Antigen-Binding Fragment Thereof

The ADCs of the present disclosure can be made in a variety of ways, using known organic chemistry reactions, conditions, and reagents, such as: (1) reacting a reactive substituent of the antibody or antigen binding fragment as described herein with the linker reagent as described herein, then reacting with the drug as described herein, such as MMAE; or (2) reacting a reactive substituent of the drug as described herein, such as MMAE, with the linker reagent as described herein, then reacting with a reactive substituent of the antibody or antigen binding fragment thereof as described herein.

In some embodiments, the reactive substituents present within an antibody or antigen binding fragment thereof as disclosed herein include N-terminal amine groups, side chain amine groups, e.g. lysine, side chain thiol groups, e.g. cysteine, and sugar hydroxyl or amino groups where the antibody is glycosylated. Reactive substituents that may be present within an antibody, or antigen binding fragment thereof, as disclosed herein include hydroxyl moieties of serine, threonine, and tyrosine residues; amino moieties of lysine residues; carboxyl moieties of aspartic acid and glutamic acid residues; and thiol moieties of cysteine residues.

In some embodiments, the reactive substituents present within an antibody or antigen binding fragment thereof as disclosed herein is a side chain thiol groups, e.g. a thiol moiety of a cysteine residue. Certain antibodies have cysteine bridges, which are reducible interchain disulphides. By treating antibodies with a reducing agent (such as DL-dithiothreitol (DTT) and tris(2-carboxyethyl)phosphine (TCEP)), they can be made reactive for conjugation with linker reagents. Each cysteine bridge will theoretically result in the formation of two reactive thiol nucleophiles. One, two, three, four, or more cysteine residues can be used to insert reactive thiol groups into an antibody (or antigen binding fragment thereof) (e.g., preparing mutant antibodies comprising one or more non-native cysteine amino acid residues).

The reaction of lysines with 2-iminothiolane (Traut's reagent), which results in the conversion of an amine to a thiol, can be used to introduce additional nucleophilic groups into antibodies. Similarly, carbohydrate groups can be chemically changed to contain one or more sulphydryl groups. The ADC is then formed by conjugation through the sulphur atom of the sulphydryl group.

In some embodiments, an ADC of Formula (I) or pharmaceutically acceptable salt thereof as described herein is prepared according to a method comprising the following steps:
(i) reacting a reactive substituent of the drug (D^{R}) as described herein with a linker reagent as described herein, to obtain a compound according to Formula (II): G^{B}-J^{B}-D^{R} as described herein; and
(ii) reacting the intermediate obtained in step (i) with the antibody or antigen binding fragment thereof as described herein to obtain the ADC of Formula (I) or pharmaceutically acceptable salt thereof.

In some embodiments, the drug is MMAE ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide). In some embodiments, the intermediate obtained in step (i) of Formula (II) has the following structure:

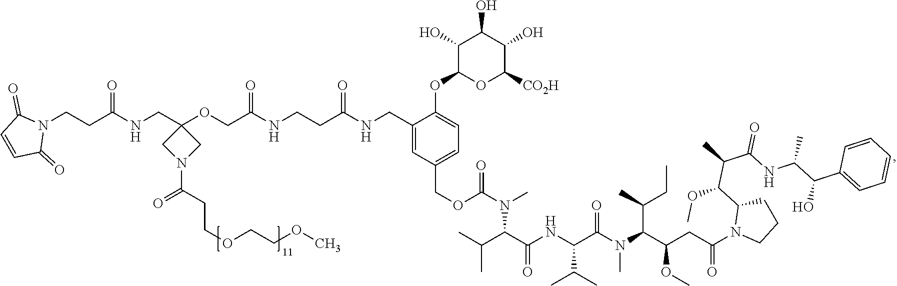

In some embodiments, step (i) of the method includes multiple steps. Examples of the synthesis of drug-linker compounds according to Formula (II) are described in Example 1.

In some embodiments, in step (ii) of the method, the antibody or antigen binding fragment thereof as described herein is conjugated to a drug, such as MMAE, for example, by partial reduction of the antibody or antigen binding fragment thereof, followed by reaction with a desired agent, with a linker moiety attached, such as a compound of structure (II). The antibody or antigen binding fragment thereof may be reduced using Tris-(2-carboxyethyl)-phosphine (TCEP) or other reducing agent to perform a similar reduction e.g. DTT. The agent with a linker moiety attached, e.g. a compound of structure (II), can then be added at a molar excess to the reduced antibody or antigen binding fragment thereof in the presence of DMSO. After conjugation, a quenching agent such as N-acetyl-L-cysteine may be added to quench unreacted agent.

Where more than one nucleophilic or electrophilic group of the antibody or antigen binding fragment thereof reacts with a linker reagent or drug-linker reagent, then the resulting product may be a mixture of ADCs with a distribution of linker-drug units attached to an antibody, e.g. 1, 2, 3, 4, etc. In some instances, separation, purification, and characterisation of homogeneous ADC, where k is a certain value from antibody with other agents, may be achieved by means such as reverse phase HPLC, electrophoresis, TFF, SEC-FPLC, SEC-prep-AKTA, CHT, HIC, spin filter centrifugation. Preparations of an ADC with a single agent loading value (k) may be isolated. The quantitative distribution of ADC in terms of k may be determined by conventional means such as UV, reverse phase HPLC, UHPLC, HIC-HPLC, SEC-HPLC, mass spectroscopy, rLCMS, ELISA assay, and electrophoresis.

Examples of G^{A} and G^{B} include, but are not limited to, the following, wherein X¹ is CH or N, h is 0 or 1, R^{K} is H or CH₃, Hal is Cl, Br or I, R^{L} is C₁₋₆ alkyl, and

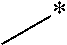 [SMILES: *C]

indicates the point of attachment to the antibody, or antigen-binding fragment thereof.

| G^{A} | G^{B} |
|---|---|
| 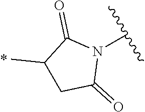 [SMILES: *N1C(=O)CC(C)C1=O] | 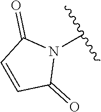 [SMILES: *N1C(=O)C=CC1=O] |
| 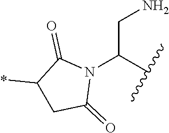 [SMILES: *C1CC(=O)N(C(*)CN)C1=O] | 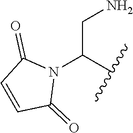 [SMILES: *C(CN)N1C(=O)C=CC1=O] |
| 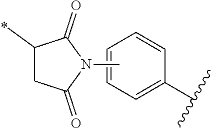 | 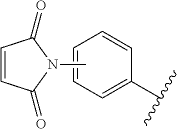 |
| 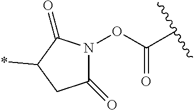 [SMILES: *C(=O)ON1C(=O)CC(*)C1=O] | 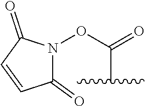 [SMILES: *C(=O)ON1C(=O)C=CC1=O] |
| 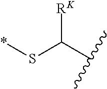 | 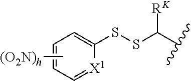 |
| 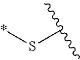 [SMILES: *S*] | 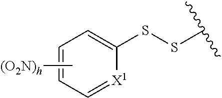 |
| 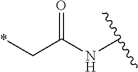 [SMILES: *CC(=O)N*] | 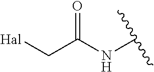 |
| 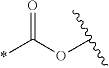 [SMILES: *OC(*)=O] | 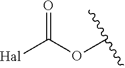 |
| 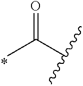 [SMILES: *C(C)=O] | 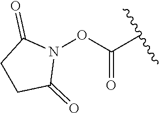 [SMILES: *C(=O)ON1C(=O)CCC1=O] |
| 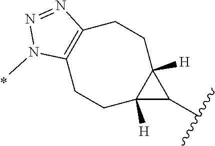 [SMILES: *C1[C@H]2CCc3nnn(C)c3CC[C@@H]12] | 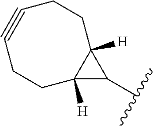 [SMILES: *C1[C@H]2CCC#CCC[C@@H]12] |
| 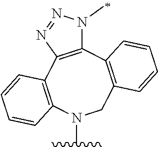 [SMILES: *N1Cc2ccccc2-c2c(nnn2C)-c2ccccc21] | 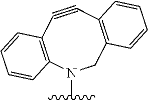 [SMILES: *N1Cc2ccccc2C#Cc2ccccc21] |
| 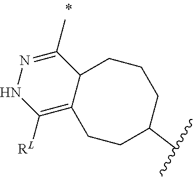 | 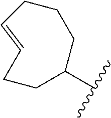 [SMILES: *C1CC/C=C/CCC1] |
| 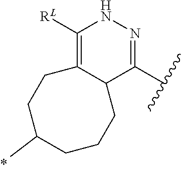 | 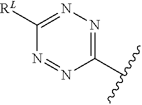 |
| 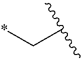 [SMILES: *C*] | 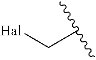 |
| 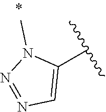 [SMILES: *c1cnnn1C] | 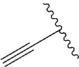 [SMILES: *C#C] |
| 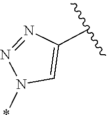 [SMILES: *c1cn(*)nn1] | |
| 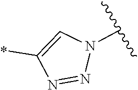 [SMILES: *c1cn(*)nn1] | 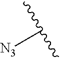 [SMILES: *N=[N+]=[N-]] |
| 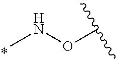 [SMILES: *ONC] | |
| 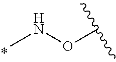 [SMILES: *ONC] | 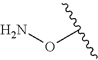 [SMILES: *ON] |
| 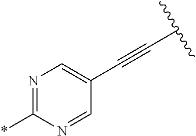 [SMILES: *C#Cc1cnc(*)nc1] | 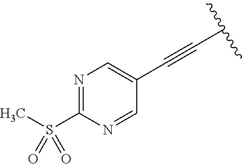 [SMILES: *C#Cc1cnc(S(C)(=O)=O)nc1] |

### Pharmaceutical Compositions

The present specification provides a pharmaceutical composition comprising (i) an antibody or antigen binding fragment thereof as described herein, or a conjugate or a pharmaceutically acceptable salt thereof as described herein, e.g. a conjugate of Formula (I) or a pharmaceutically acceptable salt thereof as described herein, and (11) a pharmaceutically acceptable excipient.

In some embodiments, there is provided a pharmaceutical composition comprising (i) an antibody or antigen binding fragment thereof as described herein and (11) a pharmaceutically acceptable excipient.

In some embodiments, there is provided a pharmaceutical composition comprising (i) a conjugate or a pharmaceutically acceptable salt thereof as described herein, e.g. a conjugate of Formula (I) or a pharmaceutically acceptable salt thereof as described herein, and (ii) a pharmaceutically acceptable excipient.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

The language “pharmaceutically acceptable excipient” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. For example, “pharmaceutically acceptable” as used herein includes compounds approved by a regulatory agency of the Federal or a state government, or listed in the U.S. Pharmacopeia, European Pharmacopeia or other generally recognized pharmacopeia for use in animals, and more particularly in humans.

The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth edition, Pharmaceutical Press, edited by Rowe, Ray C; Sheskey, Paul J; Quinn, Marian.

Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

In some embodiments, there is provided a pharmaceutical composition comprising (i) a therapeutically effective amount of an antibody or antigen binding fragment thereof as described herein, or a conjugate or a pharmaceutically acceptable salt thereof as described herein, e.g. a conjugate of Formula (I) or a pharmaceutically acceptable salt thereof as described herein, and (ii) a pharmaceutically acceptable excipient. An effective amount of an antibody or antigen binding fragment thereof as described herein, or a conjugate or a pharmaceutically acceptable salt thereof as described herein, e.g. a conjugate of Formula (I) or a pharmaceutically acceptable salt thereof as described herein, to be employed therapeutically will depend, for example, upon the therapeutic objectives, the route of administration, and the condition of the patient. The precise nature of the excipient will depend on the route of administration, which may be, e.g. oral or by injection, e.g. cutaneous, subcutaneous, or intravenous.

The disclosed compositions may be in a form suitable for oral use (for example, as tablets, lozenges, hard or soft capsules, aqueous or oily suspensions, emulsions, dispersible powders or granules, syrups or elixirs), for administration by inhalation (for example, as a finely divided powder or a liquid aerosol), for administration by insufflation (for example, as a finely divided powder) or for parenteral administration (for example, as a sterile aqueous or oily solution for intravenous, subcutaneous, intramuscular or intramuscular dosing or as a suppository for rectal dosing). The compositions may be obtained by conventional procedures well known in the art.

Methods of administering a pharmaceutical composition as described herein include, but are not limited to, parenteral administration (e.g., intradermal, intramuscular, intraperitoneal, intravenous, intra-arterial, and subcutaneous), epidural, and mucosal (e.g., intranasal and oral routes). In a specific example, a pharmaceutical composition as described herein is administered intranasally, intramuscularly, intravenously, or subcutaneously. The composition may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. Each dose may or may not be administered by an identical route of administration.

The pharmaceutical composition as described herein can be formulated for administration by any appropriate means, for example by epidermal or transdermal patches, ointments, lotions, creams, or gels; by nebulisers, vaporisers, or inhalers; by injection or infusion; or in the form of capsules, tablets, liquid solutions or suspensions in water or non-aqueous media, drops, suppositories, enemas, sprays, or powders. The most suitable route for administration in any given case will depend on the physical and mental condition of the subject, the nature and severity of the disease, and the desired properties of the formulation.

In some embodiments, the pharmaceutical composition as described herein may be in tablet, capsule, powder or liquid form for oral administration. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid cater such a gelatin.

For intravenous, cutaneous or subcutaneous injection, or injection at the site of affliction, the pharmaceutical composition as described herein will be in the form of a parenterally acceptable aqueous solution which is sterile, pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The optimum dosage and frequency of administration will depend on the particular condition being treated and its severity; the species of the patient; the age, sex, size and weight, diet, and general physical condition of the particular patient; other medication the patient may be taking; the route of administration; the formulation; and various other factors known to physicians (in the context of human patients), veterinarians (in the context of non-human patients), and others skilled in the art. For further information on Routes of Administration and Dosage Regimes the reader is referred to Chapter 25. 3 in Volume 5 of Comprehensive Medicinal Chemistry (Corwin Hansch; Chairman of Editorial Board), Pergamon Press 1990.

As used herein, the terms “subject” and “patient” are used interchangeably. The subject can be an animal. In some aspects, the subject is a mammal such as a non-human animal (e.g. cow, pig, horse, cat, dog, rat, mouse, monkey or other primate, etc.). In some aspects, the subject is a human.

### Therapeutic Uses

The present specification provides an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein, for use as a medicament. In some embodiments, there is provided an antibody or antigen binding fragment thereof as described herein for use as a medicament. In some embodiments, there is provided a pharmaceutically acceptable salt thereof as described herein, e.g. a conjugate of Formula (I) or a pharmaceutically acceptable salt thereof as described herein, for use as a medicament. In some embodiments, there is provided a pharmaceutical composition as described herein, for use as a medicament.

Also provided is an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein, for use in treating cancer, e.g. a B7H3-expressing cancer. In some embodiments, there is provided an antibody or antigen binding fragment thereof as described herein, for use in treating cancer, e.g. a B7H3-expressing cancer. In some embodiments, there is provided a conjugate or a pharmaceutically acceptable salt thereof as described herein, e.g. a conjugate of Formula (I) or a pharmaceutically acceptable salt thereof as described herein, for use in treating cancer, e.g. a B7H3-expressing cancer. In some embodiments, there is provided a pharmaceutical composition as described herein, for use in treating cancer, e.g. a B7H3-expressing cancer. In some embodiments, the cancer is a B7H3-expressing cancer. In some embodiments, the cancer is a solid tumour. In some embodiments, the cancer is lung cancer (such as non-small cell lung cancer (NSCLC), squamous cell non-small cell lung cancer (sqNSCLC), adenocarcinoma non-small cell lung cancer (adNSCLC) or small cell lung cancer (SCLC)), prostate cancer (such as castration-resistant prostate cancer (CRPC), metastatic castration-resistant prostate cancer (mCRPC), metastatic hormone-sensitive prostate cancer (mHSPC) or neuroendocrine prostate cancer (NEPC)), esophageal cancer (such as esophageal squamous-cell carcinoma (ESCC) or esophageal adenocarcinoma), bladder cancer, endometrial cancer, skin cancer (such as melanoma or cutaneous squamous cell carcinoma), sarcoma, head and neck cancer (such as head and neck squamous cell carcinoma (HNSCC), salivary gland cancer, oral cancer, or oral squamous cell carcinoma), breast cancer, liver cancer (such as hepatocellular carcinoma (HCC) or intrahepatic cholangiocarcinoma), ovarian cancer, colorectal cancer, pancreatic cancer, kidney cancer (such as renal cell carcinoma (RCC)), biliary tract cancer (such as cholangiocarcinoma or intrahepatic cholangiocarcinoma), gastric cancer, thyroid cancer, cervical cancer, brain cancer (such as glioma) or adrenal cancer (such as adrenocortical carcinoma). In some embodiments, the cancer is prostate cancer. In other embodiments, the cancer is squamous non-small cell lung cancer.

Also provided is a method of treating a disease or disorder in a patient in need thereof comprising administering to the patient a therapeutically effective amount of an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein. In some embodiments, the disease or disorder is cancer, e.g. a B7H3-expressing cancer. In some embodiments, there is provided a method of treating a disease or disorder in a patient in need thereof comprising administering to the patient a therapeutically effective amount of an antibody or antigen binding fragment thereof as described herein, optionally wherein the disease or disorder is cancer, e.g. a B7H3-expressing cancer. In some embodiments, there is provided a method of treating a disease or disorder in a patient in need thereof comprising administering to the patient a therapeutically effective amount of a conjugate or a pharmaceutically acceptable salt thereof as described herein, e.g. a conjugate of Formula (I) or a pharmaceutically acceptable salt thereof as described herein, optionally wherein the disease or disorder is cancer, e.g. a B7H3-expressing cancer. In some embodiments, there is provided a method of treating a disease or disorder in a patient in need thereof comprising administering to the patient a therapeutically effective amount of a pharmaceutical composition as described herein, optionally wherein the disease or disorder is cancer, e.g. a B7H3-expressing cancer. In some embodiments, the cancer is a B7H3-expressing cancer. In some embodiments, the cancer is a solid tumour. In some embodiments, the cancer is lung cancer (such as non-small cell lung cancer (NSCLC), squamous cell non-small cell lung cancer (sqNSCLC), adenocarcinoma non-small cell lung cancer (adNSCLC) or small cell lung cancer (SCLC)), prostate cancer (such as castration-resistant prostate cancer (CRPC), metastatic castration-resistant prostate cancer (mCRPC), metastatic hormone-sensitive prostate cancer (mHSPC) or neuroendocrine prostate cancer (NEPC)), esophageal cancer (such as esophageal squamous-cell carcinoma (ESCC) or esophageal adenocarcinoma), bladder cancer, endometrial cancer, skin cancer (such as melanoma or cutaneous squamous cell carcinoma), sarcoma, head and neck cancer (such as head and neck squamous cell carcinoma (HNSCC), salivary gland cancer, oral cancer, or oral squamous cell carcinoma), breast cancer, liver cancer (such as hepatocellular carcinoma (HCC) or intrahepatic cholangiocarcinoma), ovarian cancer, colorectal cancer, pancreatic cancer, kidney cancer (such as renal cell carcinoma (RCC)), biliary tract cancer (such as cholangiocarcinoma or intrahepatic cholangiocarcinoma), gastric cancer, thyroid cancer, cervical cancer, brain cancer (such as glioma) or adrenal cancer (such as adrenocortical carcinoma). In some embodiments, the cancer is prostate cancer. In other embodiments, the cancer is squamous non-small cell lung cancer.

Also provided is the use of an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein, in the manufacture of a medicament for the treatment of cancer, e.g. a B7H3-expressing cancer, in a subject. In some embodiments, there is provided the use of an antibody or antigen binding fragment thereof as described herein in the manufacture of a medicament for the treatment of cancer, e.g. a B7H3-expressing cancer, in a subject. In some embodiments, there is provided the use of a conjugate or a pharmaceutically acceptable salt thereof as described herein, e.g. a conjugate of Formula (I) or a pharmaceutically acceptable salt thereof as described herein, in the manufacture of a medicament for the treatment of cancer, e.g. a B7H3-expressing cancer, in a subject. In some embodiments, there is provided the use of a pharmaceutical composition as described herein in the manufacture of a medicament for the treatment of cancer, e.g. a B7H3-expressing cancer, in a subject. In some embodiments, the cancer is a B7H3-expressing cancer. In some embodiments, the cancer is a solid tumour. In some embodiments, the cancer is lung cancer (such as non-small cell lung cancer (NSCLC), squamous cell non-small cell lung cancer (sqNSCLC), adenocarcinoma non-small cell lung cancer (adNSCLC) or small cell lung cancer (SCLC)), prostate cancer (such as castration-resistant prostate cancer (CRPC), metastatic castration-resistant prostate cancer (mCRPC), metastatic hormone-sensitive prostate cancer (mHSPC) or neuroendocrine prostate cancer (NEPC)), esophageal cancer (such as esophageal squamous-cell carcinoma (ESCC) or esophageal adenocarcinoma), bladder cancer, endometrial cancer, skin cancer (such as melanoma or cutaneous squamous cell carcinoma), sarcoma, head and neck cancer (such as head and neck squamous cell carcinoma (HNSCC), salivary gland cancer, oral cancer, or oral squamous cell carcinoma), breast cancer, liver cancer (such as hepatocellular carcinoma (HCC) or intrahepatic cholangiocarcinoma), ovarian cancer, colorectal cancer, pancreatic cancer, kidney cancer (such as renal cell carcinoma (RCC)), biliary tract cancer (such as cholangiocarcinoma or intrahepatic cholangiocarcinoma), gastric cancer, thyroid cancer, cervical cancer, brain cancer (such as glioma) or adrenal cancer (such as adrenocortical carcinoma). In some embodiments, the cancer is prostate cancer. In other embodiments, the cancer is squamous non-small cell lung cancer.

The present specification also provides the use of an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein, in combination with a further therapeutic agent. In some embodiments, the further therapeutic agent is an anti-tumour agent.

Accordingly, there is provided an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein, for use in treating cancer, wherein the antibody or antigen binding fragment thereof, the conjugate or a pharmaceutically acceptable salt thereof, e.g. the conjugate of Formula (I) or a pharmaceutically acceptable salt thereof, or the pharmaceutical composition, is used in combination with a further therapeutic agent. In some embodiments, the further therapeutic agent is an anti-tumour agent.

There is also provided a method of treating a cancer in a patient in need thereof comprising administering to the patient a therapeutically effective amount of an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein, wherein the antibody or antigen binding fragment thereof, the conjugate or a pharmaceutically acceptable salt thereof, e.g. the conjugate of Formula (I) or a pharmaceutically acceptable salt thereof, or the pharmaceutical composition, is used in combination with a further therapeutic agent. In some embodiments, the further therapeutic agent is an anti-tumour agent.

There is also provided the use of an antibody or antigen binding fragment thereof as described herein, a conjugate or a pharmaceutically acceptable salt thereof as described herein, e.g. a conjugate of Formula (I) or a pharmaceutically acceptable salt thereof as described herein, or a pharmaceutical composition as described herein, in the manufacture of a medicament for the treatment of cancer in a subject, wherein the antibody or antigen binding fragment thereof, the conjugate or a pharmaceutically acceptable salt thereof, e.g. the conjugate of Formula (I) or a pharmaceutically acceptable salt thereof, or the pharmaceutical composition, is used in combination with a further therapeutic agent. In some embodiments, the further therapeutic agent is an anti-tumour agent.

In embodiments, the antibody or antigen binding fragment thereof, the conjugate or pharmaceutically acceptable salt thereof, e.g. conjugate of Formula (I) or pharmaceutically acceptable salt thereof, or the pharmaceutical composition, and the further therapeutic agent, e.g. anti-tumour agent, are administered to a patient in need thereof simultaneously, separately or sequentially. In some embodiments, the antibody or antigen binding fragment thereof, the conjugate or pharmaceutically acceptable salt thereof, e.g. conjugate of Formula (I) or pharmaceutically acceptable salt thereof, or the pharmaceutical composition, and the further therapeutic agent, e.g. anti-tumour agent, are jointly effective in producing an anti-cancer effect.

The present specification provides a kit comprising a conjugate or pharmaceutically acceptable salt thereof as described herein, e.g. a conjugate of Formula (I) or pharmaceutically acceptable salt thereof as described herein, or a pharmaceutical composition as described herein, and instructions to administer the conjugate or pharmaceutically acceptable salt thereof or the pharmaceutical composition to a subject in need of treatment. In some embodiments, the kit is for treating cancer.

There is also provided a pharmaceutical or diagnostic pack or kit comprising one or more containers filled with one or more of the ingredients of the pharmaceutical compositions as disclosed herein. Optionally associated with such container(s) can be a notice in the form prescribed by a governmental agency regulating the manufacture, use or sale of pharmaceuticals or biological products, which notice reflects approval by the agency of manufacture, use or sale for human administration, e.g., an authorisation number.

Also provided herein is an article of manufacture that includes a container in which a pharmaceutical composition comprising a conjugate or pharmaceutically acceptable salt thereof as described herein and a packaging insert or label indicating that the composition can be used to treat cancer. In some embodiments, there is provided a kit for treating and/or preventing cancer, the kit comprising the conjugate or pharmaceutically acceptable salt thereof optionally in combination with a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the conjugate or pharmaceutically acceptable salt thereof. In another embodiment, the kit comprises the conjugate or pharmaceutically acceptable salt thereof contained within a container or an IV bag. In another embodiment, the container or IV bag is a sterile container or a sterile IV bag. In another embodiment, the conjugate or pharmaceutically acceptable salt thereof is formulated into a pharmaceutical composition contained within a (sterile) container or contained within a (sterile) IV bag. In a further embodiment, the kit further comprises instructions for use.

In another embodiment, a kit for treating cancer, is provided, wherein the kit includes a conjugate or pharmaceutically acceptable salt thereof as described herein and instructions to administer the conjugate or pharmaceutically acceptable salt thereof to a subject in need of treatment. The subject in need is specifically defined in the kit as someone of a specific higher risk group defined by epidemiological data, risk stratification data from the person's health records, risk stratification by the genotype of certain genes of the individual or the presence of certain biomarkers in the person's blood or other tissue sample. Where the risk stratification involves another pharmaceutical or diagnostic pack or kit, the combined product will act as a linked diagnostic/prognostic and treatment kit.

### EXAMPLES

### Example 1: Synthesis of Linker-Payload

Methods for the syntheses of the linker payloads described herein are disclosed, for example, in PCT Application No. PCT/EP2024/072931, filed 14 Aug. 2024 & published as International Publication Pamphlet WO2025/036947, the contents of which are hereby incorporated by reference for all purposes.

General Information Reagents and solvents (all anhydrous HPLC-grade) were obtained from commercial suppliers and used without any further purification unless otherwise stated. All reagents were weighed and handled in air unless otherwise stated. Brine refers to a saturated solution of NaCl. Concentration under reduced pressure refers to the use of a rotary evaporator. Pegylated reagents were obtained from PUREPEG. Mc-vc-PAB-MMAE (maleimidocaproyl-valine-citrulline-p-aminobenzoyloxycarbonyl-MMAE, CAS:646502-53-6) was obtained from Med Chem Express and will be referred to as LP1. In general Examples and Intermediate compounds are named using CHEMDRAW PROFESSIONAL version 22.2.0.3300 from PERKINELMER.

### List of Abbreviations

ADC: Antibody Drug Conjugate; All: Allyl; Alloc: Allyloxycarbonyl; TFA: trifluoroacetic acid; FA: Formic acid; FC: flash chromatography; FMOC: fluorenylmethyloxycarbonyl; DCM: dichloromethane; DBA: Dibenzylideneacetone; DIPEA: Di isopropyl ethylamine; DMF: N,N-dimethylformamide; DMSO: dimethylsulfoxide; dppf: 1,1′-Bis(diphenylphosphino)ferrocene; ESI: Electrospray ionisation; HFIP: 1,1,1,3,3,3-Hexafluoro-2-propanol; HATU: 1-[Bis(dimethylamino)methylene]-1H-1,2,3-triazolo[4,5-b]pyridinium 3-oxid hexafluorophosphate; HOPO: 1-Hydroxy-2-pyridone; mAb: Monoclonal antibody; MS Molecular sieves; RT: Retention time; Tf triflate; THF: Tetra hydro furan; UHPLC: Ultra-high performance liquid chromatography; V: Volumes.

### Flash Column Chromatography

### Method A

Flash chromatography was performed using a BIOTAGE ISOLERA and fractions checked for purity using thin-layer chromatography (TLC). TLC was performed using MERCK KIESELGEL 60 F254 silica gel, with fluorescent indicator on aluminium plates. Visualisation of TLC was achieved with UV light. chromatography solvents were bought and used without further purification from VWR U.K. This method was applied to Intermediates 5-8 and 22-23, unless stated differently.

### Method B

Chromatographic purification of products was accomplished with a CHEETAH MP200 system with integrated UV detection, using column chromatography on (A) silica gels (40-60 μm), eluted with petroleum ether and ethyl acetate or dichloromethane and methanol, (B) C18 spherical (20-35 μm), eluted with water and acetonitrile with formic acid (0.1%) or trifluoracetic acid (0.05%) or ammonium formate (10 mmol) modifier. This method was applied to all other not stated in FC method A, unless stated differently.

### LC/MS Conditions

### Method A

Positive mode electrospray mass spectrometry was performed using a WATERS ACQUITY H-CLASS SQD2. Mobile phases used were solvent A (water with 0.1% formic acid) and solvent B (acetonitrile with 0.1% formic acid). Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Detection was at 254 nm. Columns: WATERS ACQUITY UPLC BEH SHIELD RP18 1.7 μm 2.1×50 mm at 50° C. fitted with WATERS ACQUITY UPLC BEH SHIELD RP18 VANGUARD Pre-column, 130A, 1.7 μm, 2.1 mm×5 mm. This method was applied to Intermediates 20 and 21, unless stated differently.

### Method B

Samples were analysed using a QEXACTIVE ORBITRAP (THERMO) coupled to a DIONEX ULTIMATE 3000 UPLC (THERMO) equipped with an ACQUITY UPLC CSH C18 RP column (2.1 mm×50 mm, 1.7 μm particle (Waters)). Autosampler and column temperatures were set to 50° C. and 10° C. respectively. Five μL of 200 uM sample dissolved in 1:1 v/v acetonitrile/water were injected on column and separated over 9 minutes (reduced samples) or 10 minutes (intact samples) using a gradient of 5-100% mobile phase B (0.1% FA in acetonitrile) in mobile phase A (0.1% TFA in water) at 0.5 mL/min. Settings for heated electrospray source were as follows: sheath gas flow rate 50, auxiliary gas flow rate 15 and spray voltage 3.5 kV. RF level was set to 50. Capillary temperature and auxiliary gas heater temperature were set to 350° C. and 400° C. respectively. UV trace was recorded at 223 nm and 330 nm. Full scan mass spectra were acquired in positive mode with resolution of 70000, AGC target set to 3E6, maximum injection time set to 200 ms and mass range of 150-2000 m/z. Fragmentation spectra were obtained using all ion fragmentation mode for 133-2000 m/z with stepped normalized collision energy of 15, 28 and 45. The acquired data was analysed with THERMO XCALIBUR QUAL BROWSER (version 3.1.66.10). This method was used for Intermediates 5-8 and 22-23 and LP2, unless stated differently.

### Method C

Samples were analysed using a SHIMADZU LCMS-2020 with electrospray ionization in positive ion detection mode with 20ADXR pump, SIL-20ACXR autosampler, CTO-20AC column oven, M20A PDA Detector and LCMS 2020 MS detector. LC was run in two set ups: 1) Halo C18 column (2.0 μm 3.0×30 mm) in combination with a gradient (5-100% B in 1.2 minutes) of water and FA (0.1%) (A) and CH₃CN and FA (0.1%) (B) at a flow rate of 1.5 mL/min; 2) POROSHELL HPH C18 column (2.7 μm 3.0×50 mm) in combination with a gradient (5-95% B in 2 minutes) of aqueous 46 mM ammonium carbonate/ammonia buffer at pH 10 (A) and MeCN (B) at a flow rate of 1.2 mL/min; 3) Halo C18 column (2.0 μm 3.0×30 mm) in combination with a gradient (5-95% B in 2 minutes) of water and TFA (0.05%) (A) and CH₃CN and TFA (0.05%) at a flow rate of 1.5 mL/min (B). The Column Oven (CTO-20AC) temperature was 40.0° C. The injection volume was 1 μl. PDA (SPD-M20A) detection was in the range 190-400 nm. The MS detector, which was configured with electrospray ionization as ionizable source; Acquisition mode: Scan; Nebulizing Gas Flow:1.5 L/min; Drying Gas Flow:15 L/min; Detector Voltage: Tuning Voltage ±0.2 kv; DL Temperature: 250° C.; Heat Block Temperature: 250° C.; Scan Range: 90.00-900.00 m/z. This method was applied to all other intermediates not stated in LCMS method A or B, unless stated differently.

### HPLC Conditions

### Method A

Reverse-phase ultra-fast high-performance liquid chromatography (UFLC) was carried out on a SHIMADZU PROMINENCE machine using a PHENOMENEX GEMINI NX 5p C18 column (at 50° C.) dimensions: 150×21.2 mm. Eluents used were solvent A (H₂O with 0.1% formic acid) and solvent B (CH₃CN with 0.1% formic acid) or solvent A (H₂O with 0.1% trifluoroacetic acid acid) and solvent B (CH₃CN with 0.1% trifluoroacetic acid), used for LP2, unless stated differently.

### Method B

Preparative HPLC was performed with a WATERS MASSLYNX system with integrated MS detection and equipped with Prep C18 OBD 5 μm 30×150 mm columns from XBRIDGE or XSELECT CSH. Alternatively, GILSON GX-281 with integrated UV detection was used, equipped with either XBRIDGE or SUNFIRE C18 10 μm, 19×150 ID or 19×250 mm. As eluent (acidic) gradients of water/MeCN/FA acid (95/5/0.1) or water/0.05% TFA (A) and MeCN/MeOH (B) or 0.05% ammonia in water/10 mmol NH₄HCO₃ (A) and MeCN/MeOH (B) were applied. This method was used for intermediate 20.

### Synthesis of Intermediate 8

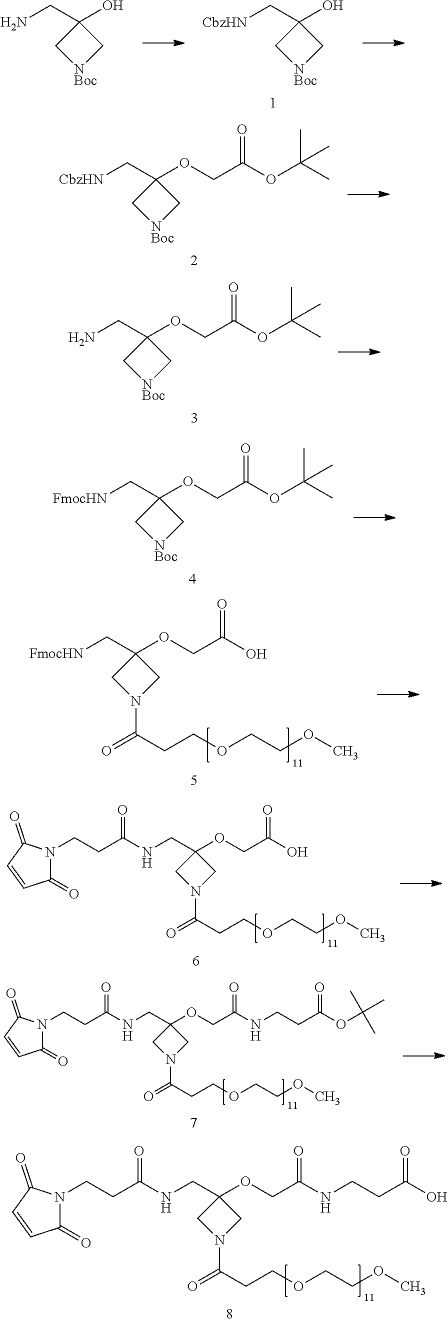

### Step 1

tert-butyl 3-(aminomethyl)-3-hydroxyazetidine-1-carboxylate (10 g, 49.44 mmol) was added to N-ethyl-N-isopropylpropan-2-amine (19.17 g, 148.33 mmol) and N-(benzyloxycarbonyloxy)succinimide (14.79 g, 59.33 mmol) in THF (150 mL) under nitrogen. The resulting mixture was stirred at 25° C. for 3 hours. The solvent was removed under reduced pressure. The crude product was purified by flash silica chromatography, elution gradient 0 to 10% MeOH in DCM. Pure fractions were evaporated to dryness to afford tert-butyl 3-((((benzyloxy)carbonyl)amino)methyl)-3-hydroxyazetidine-1-carboxylate (Intermediate 1, 13.00 g, 78%) as a yellow oil. m/z (ES+), [M+H]+=337; TFA, HPLC tR=0.903 min.

### Step 2

Intermediate 1 (13 g, 38.65 mmol) was added to tert-butyl 2-bromoacetate (9.05 g, 46.38 mmol) and sodium hydride (1.855 g, 77.29 mmol) in THF (150 mL) under nitrogen at 0° C. The resulting mixture was stirred at 25° C. for 2 hours. The reaction mixture was quenched with water (50 mL), extracted with EtOAc (3×150 mL), the top layer was dried over Na₂SO₄, filtered and evaporated to afford yellow oil. The crude product was purified by flash silica chromatography, elution gradient 0 to 50% EtOAc in petroleum ether. Pure fractions were evaporated to dryness to afford tert-butyl 3-((((benzyloxy)carbonyl)amino)methyl)-3-(2-(tert-butoxy)-2-oxoethoxy)azetidine-1-carboxylate (Intermediate 2, 9.00 g, 51.7%) as a colourless oil. m/z (ES+), [M+H]+=451; TFA, HPLC tR=1.125 min.

### Step 3

Intermediate 2 (2, 9 g, 19.98 mmol) was added to ROYER™ Palladium Catalyst Powder (CAS 7440-05-3, 6.07 g, 19.98 mmol) in THF (120 mL) under hydrogen. The resulting mixture was stirred at 25° C. for 6 hours. The reaction mixture was filtered through silica and washed with THF (3×50 mL). The solvent was removed under reduced pressure to afford tert-butyl 3-(aminomethyl)-3-(2-(tert-butoxy)-2-oxoethoxy)azetidine-1-carboxylate (Intermediate 3, 6.00 g, 85%) as a yellow oil. m/z (ES+), [M+H]+=317; TFA, HPLC tR=0.882 min.

### Step 4

Intermediate 3 (5 g, 15.80 mmol) was added to 9-fluorenylmethyl chloroformate (6.13 g, 23.70 mmol) and N,N-diisopropylethylamine (6.13 g, 47.41 mmol) in THF (50 mL) under nitrogen. The resulting mixture was stirred at 25° C. for 3 hours. The solvent was removed under reduced pressure. The crude product was purified by flash silica chromatography, elution gradient 0 to 50% EtOAc in petroleum ether. Pure fractions were evaporated to dryness to afford tert-butyl 3-(((((9H-fluoren-9-yl)methoxy)carbonyl)amino)methyl)-3-(2-(tert-butoxy)-2-oxoethoxy)azetidine-1-carboxylate (4, 3.66 g, 43.0%) as a white solid. m/z (ES+), [M+H]+=539; TFA, HPLC tR=1.230 min.

### Step 5

To Intermediate 4 (615 mg, 1.14 mmol) in DCM (4 mL) was added TFA (4 mL). The reaction mixture was stirred at room temperature for 1.5 h. The reaction mixture was concentrated under reduced pressure to give a crude product which was used without further purification. A solution of 1-[(38-oxo-2,5,8,11,14,17,20,23,26,29,32,35-dodecaoxaoctatriacontan-38-yl)oxy]-2,5-pyrrolidinedione (708 mg, 1.03 mmol) and DIPEA (0.397 mL, 2.28 mmol) in DMF (5 mL) was added to crude 2-((3-(((((9H-fluoren-9-yl)methoxy)carbonyl)amino)methyl)azetidin-3-yl)oxy)acetic acid (436 mg, 1.14 mmol). The reaction mixture was stirred at room temperature for 1.5 h. The reaction mixture was purified by reverse phase column chromatography (5-60% MeCN in water +0.1% FA) to give 2-((3-(((((9H-fluoren-9-yl)methoxy)carbonyl)amino)methyl)-1-(2,5,8,11,14,17,20,23,26,29,32,35-dodecaoxaoctatriacontan-38-oyl)azetidin-3-yl)oxy)acetic acid (Intermediate 5, 654 mg, 66.5%) as a colourless oil. LCMS (15 min): 6.43 min; 954.7 [M+H]+

### Step 6

To Intermediate 5 (615 mg, 1.14 mmol) in DCM (4 mL) was added diethylamine (4 mL). The reaction mixture was stirred at room temperature for 1.5 h. The reaction mixture was concentrated under reduced pressure to give a crude product which was used without further purification. To crude 2-((3-(aminomethyl)-1-(2,5,8,11,14,17,20,23,26,29,32,35-dodecaoxaoctatriacontan-38-oyl)azetidin-3-yl)oxy)acetic acid (504 mg, 0.69 mmol) was added 2,5-dioxopyrrolidin-1-yl 3-(2,5-dioxo-2,5-dihydro-1H-pyrrol-1-yl)propanoate (367 mg, 1.38 mmol) and N-ethyl-N-isopropylpropan-2-amine (0.240 mL, 1.38 mmol). The reaction mixture was stirred at room temperature for 2 h. The reaction mixture was purified by reverse phase chromatography (5-50% MeCN in water +0.1% FA) to give 2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetic acid (Intermediate 6, 280 mg, 46.0%) as a colourless oil. LCMS (15 min): 4.66 min; 881. [M+H]+

### Step 7

To the stirred solution of Intermediate 6 (lg, 1.13 mmol) in DCM (20 mL) was added tert-butyl 3-aminopropanoate hydrochloride (410 mg, 2.3 mmol) to the flask. Then added HATU (860 mg, 2.3 mmol), DIEA (590 mg, 4.6 mmol.) and DMF (5 ml) to the flask and stirred at room temperature for 2 hours. The reaction mixture was diluted with EtOAc (20 mL) and then washed with saturated sodium bicarbonate aqueous solution (10 mL), aqueous citric acid (1M, 10 mL), brine (10 mL). After dried over sodium sulfate, and filtered, the solution was concentrated to dryness to afford tert-butyl 3-(2-((3-((3-(2,5-dioxo-2,5-dihydro-1H-pyrrol-1-yl) propanamido) methyl)-1-(2,5,8,11,14,17,20,23,26,29,32,35-dodecaoxaoctatriacontan-38-oyl) azetidin-3-yl) oxy) acetamido)propanoate was obtained as an oil (Intermediate 7, 500 mg, 43.7%). MS ESI: [M+H]+m/z 1009.4.

### Step 8

To the solution of Intermediate 7 (550 mg, 0.5 mmol) in the mixed solvent of DCM (11 mL) was added TFA (5.5 mL) at 20° C. The resulting solution was stirred at room temperature for 2 hours. After the starting material was consumed completely, the reaction solution was concentrated to dryness under vacuum. The crude residue was purified by silica gel column with 0-15% MeOH in DCM to afford 3-(2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetamido)propanoic acid as an oil (Intermediate 8, 200 mg, 38.5%). MS ESI: [M+H]+m/z 953.4.

### Intermediate 9

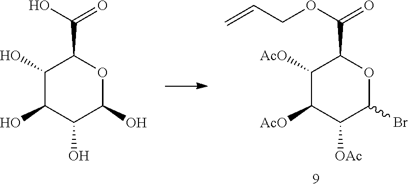 [SMILES: O=C(O)[C@H]1O[C@@H](O)[C@H](O)[C@@H](O)[C@@H]1O ; C=CCOC(=O)[C@H]1OC(Br)[C@H](OC(C)=O)[C@@H](OC(C)=O)[C@@H]1OC(C)=O]

2,3,4,6,7,8,9,10-octahydropyrimido[1,2-a]azepine (26.5 ml, 177.35 mmol) was added dropwise to a 1-L round bottom flask containing (2S,3S,4S,5R,6R)-3,4,5,6-tetrahydroxytetrahydro-2H-pyran-2-carboxylic acid (31.3 g, 161.22 mmol) in DMF (100 ml) at 21° C. Next, 3-bromoprop-1-ene (16.72 ml, 193.47 mmol) was added to the reaction mixture dropwise over 10 minutes and the reaction was stirred at 21° C. for 24 hours. Reaction mixture was cooled to 0° C. and treated with pyridine (104 mL, 1289.60 mmol). Acetic anhydride (244 mL, 2579.20 mmol) was next added to the reaction mixture. The reaction was warmed up to room temperature and run for 2 hours at 21° C. Reaction mixture concentrated under reduced vacuum and the remaining pyridine was azeotropically removed with toluene (1×100 mL). Crude material was diluted with DCM (65 mL) and cooled to 0° C. 30% hydrobromic acid in acetic acid (175 mL, 3226.03 mmol) was next added to the reaction mixture at 0° C. The reaction was warmed up to room temperature and run for 2 hours 30 minutes at 21° C. Solvent was evaporated then the compound was purified by normal phase flash column chromatography to afford (2S,3S,4S,5R,6R)-2-((allyloxy)carbonyl)-6-bromotetrahydro-2H-pyran-3,4,5-triyl triacetate (Intermediate 9, 33 g, 48% yield) as a beige translucent material. LCMS (ESI) m/z 445.0 (M+Na)+.

### Intermediate 10

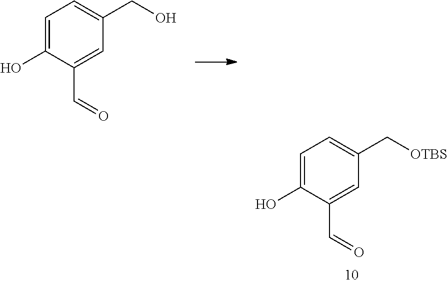 [SMILES: O=Cc1cc(CO)ccc1O ; CC(C)(C)[Si](C)(C)OCc1ccc(O)c(C=O)c1]

TBS-Cl (20.80 g, 138.02 mmol) in DCM (25 mL) was added dropwise to 1H-imidazole (17.90 g, 262.90 mmol) and 2-hydroxy-5-(hydroxymethyl)benzaldehyde (20 g, 131.45 mmol) in DCM (500 mL) at 0° C. over a period of 2 hours under nitrogen. The resulting mixture was stirred at 0° C. for 2 hours. The reaction mixture was quenched with water (500 mL), extracted with DCM (2×300 mL) and the organic layer was dried over Na₂SO₄, filtered and evaporated to afford 5-(((tert-butyldimethylsilyl)oxy)methyl)-2-hydroxybenzaldehyde (Intermediate 10, 35.0 g, 100%) as a colourless material. m/z (ES+), [M+Na]+=289; NH₄HCO₃, HPLC tR=1.505 min.

### Intermediate 11

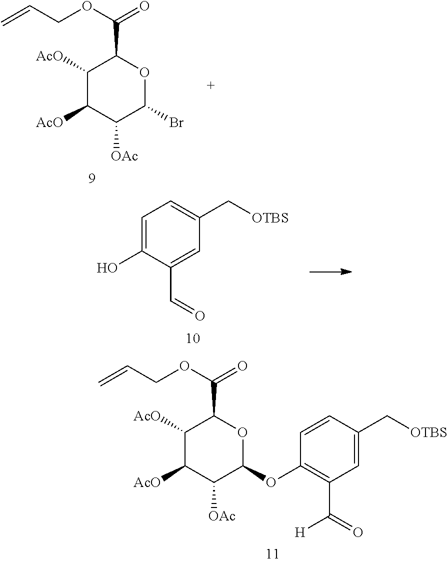 [SMILES: C=CCOC(=O)[C@H]1O[C@H](Br)[C@H](OC(C)=O)[C@@H](OC(C)=O)[C@@H]1OC(C)=O ; CC(C)(C)[Si](C)(C)OCc1ccc(O)c(C=O)c1 ; C=CCOC(=O)[C@H]1O[C@@H](Oc2ccc(CO[Si](C)(C)C(C)(C)C)cc2C=O)[C@H](OC(C)=O)[C@@H](OC(C)=O)[C@@H]1OC(C)=O]

To a vacuum-dried 500 mL round-bottom flask was added molecular sieves (4 Å beads, 5.0 g), silver oxide (29.2 g, 125.8 mmol) and acetonitrile (150 mL), producing a black slurry. To this slurry was added a solution of Intermediate 9 (10.7 g, 25.2 mmol) in acetonitrile (50 mL) over 20 min followed by the addition of Intermediate 10 (13.6 g, 51.1 mmol) in acetonitrile (50 mL) in one portion. The resulting mixture was stirred vigorously at 20° C. for 16 h. After 16 h, the reaction mixture was filtered through a 5-cm pad of Celite and rinsed with dichloromethane (3×25 mL). Solvent was evaporated then the compound was purified by normal phase flash column chromatography to afford (2S,3S,4S,5R,6S)-2-((allyloxy)carbonyl)-6-(4-(((tert-butyldimethylsilyl)oxy)methyl)-2-formylphenoxy)tetrahydro-2H-pyran-3,4,5-triyl triacetate as a white material (Intermediate 11, 5.2 g, 34% yield). LCMS (ESI) m/z 626.3 (M+NH₄)+.

### Intermediate 12

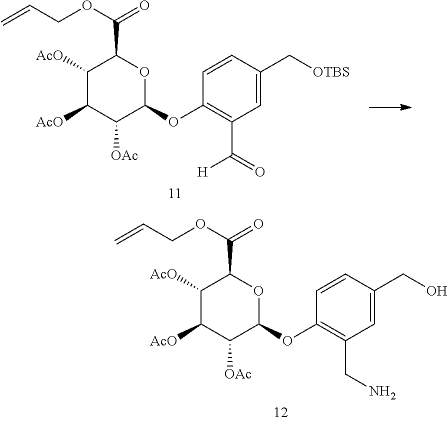 [SMILES: C=CCOC(=O)[C@H]1O[C@@H](Oc2ccc(CO[Si](C)(C)C(C)(C)C)cc2C=O)[C@H](OC(C)=O)[C@@H](OC(C)=O)[C@@H]1OC(C)=O ; C=CCOC(=O)[C@H]1O[C@@H](Oc2ccc(CO)cc2CN)[C@H](OC(C)=O)[C@@H](OC(C)=O)[C@@H]1OC(C)=O]

To a solution of Intermediate 11 (5.2 g, 8.6 mmol) in acetonitrile (40 mL) was added tert-butyl carbamate (3.8 g, 32.3 mmol), trifluoroacetic acid (2.0 mL, 25.9 mmol), and triethylsilane (4.1 mL, 25.8 mmol). Stirred for 2 h at 20° C. then solvent was evaporated. To the resulting colorless oil was added 1,4-dioxane (8 mL) and HCl (4.0 M in 1,4-dioxane, 50 mL, 200 mmol). The mixture was stirred at 20° C. for 30 min the solvent was evaporated. The resulting white powder was dissolved in DMSO (3 mL) then passed through cation-exchange resin pre-treated with methanol (WATERS PORAPAK CX). The desired compound was eluted off the resin with methanol to afford (2S,3S,4S,5R,6S)-2-((allyloxy)carbonyl)-6-(2-(aminomethyl)-4-(hydroxymethyl)phenoxy)tetrahydro-2H-pyran-3,4,5-triyl triacetate as a white material (Intermediate 12, 2.5 g, 80% over 2 steps). LCMS (ESI) m/z 496.5 (M+H)+.

### Intermediate 13

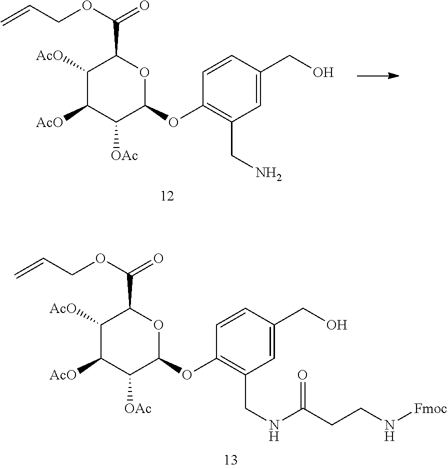

To a stirred reactor containing Intermediate 12 (2.1 kg, 90.5% w/w, 3.57 mol) and acetonitrile (19 L) was added Fmoc-β-alanine (1.11 kg, 3.57 mol). The stirred mixture was cooled to 0° C. To this was added hexafluorophosphate azabenzotriazole tetramethyl uronium (1.36 kg, 3.57 mol) and N,N-diispropylethylamine (0.92 kg, 7.14 mol), and stirred for 4 hours, maintaining the temperature at 0° C. Water (19 L) and ethyl acetate (19 L) was added to the stirred mixture. The organic phase was separated and concentrated to ~19 L under vacuum. Ethyl acetate (28.5 L) was added to the concentrated solution and stirred at 20-25° C. for 18 hours. The resulting suspension was filtered, the cake washed with ethyl acetate (3.87 L), and dried under vacuum to (2S,3R,4S,5S,6S)-2-(2-((3-((((9H-fluoren-9-yl)methoxy)carbonyl)amino)propanamido)methyl)-4-(hydroxymethyl)phenoxy)-6-((allyloxy)carbonyl)tetrahydro-2H-pyran-3,4,5-triyl triacetate (Intermediate 13, 1.6 kg, 99% w/w, 5 6%). LCMS m/z 789 [M+H]⁺.

### Intermediate 14

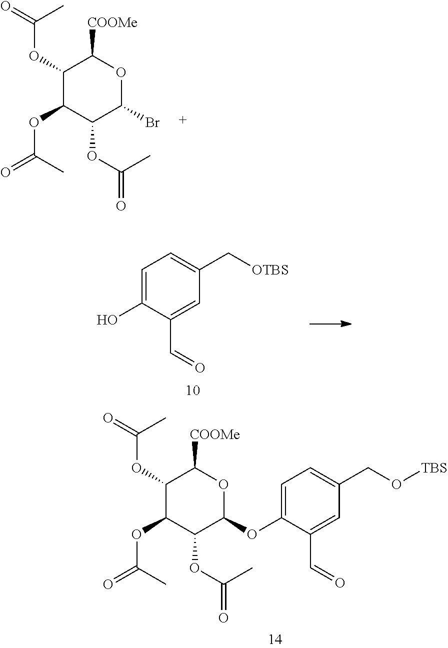

Silver(I) oxide p.a. (52.2 g, 225.22 mmol) was added to 4 Å Molecular Sieves (30 g), (2R,3R,4S,5S,6S)-2-bromo-6-(methoxycarbonyl)tetrahydro-2H-pyran-3,4,5-triyl triacetate (44.7 g, 112.61 mmol), Intermediate 10 (15 g, 56.30 mmol) in MeCN (500 mL) at 0° C. over a period of 2 hours under nitrogen. The resulting mixture was stirred at 25° C. for 16 hours. The mixture was filtered through a Celite pad. The crude product was purified by flash silica chromatography, elution gradient 0 to 30% EtOAc in petroleum ether. Pure fractions were evaporated to dryness to afford (2S,3R,4S,5S,6S)-2-(4-(((tert-butyldimethylsilyl)oxy)methyl)-2-formylphenoxy)-6-(methoxycarbonyl)tetrahydro-2H-pyran-3,4,5-triyl triacetate (Intermediate 14) as a pale yellow material. m/z (ES+), [M+Na]+=605; TFA, HPLC tR=1.071 min.

### Intermediate 15

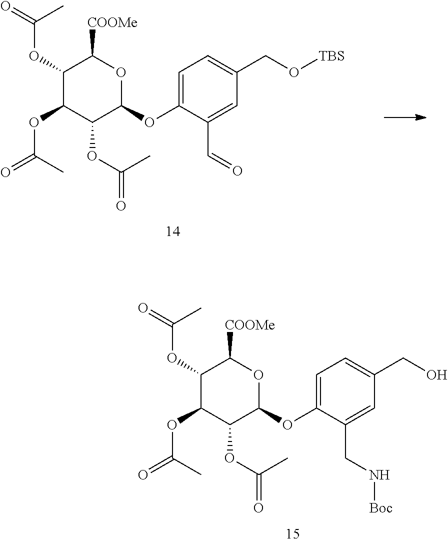

TFA (8 mL) was added to Et₃SiH (32 mL), tert-butyl carbamate (15.28 g, 130.43 mmol) and Intermediate 14 (20 g, 34.32 mmol) in MeCN (200 mL) at 0° C. under nitrogen. The resulting mixture was stirred at 0° C. for 16 hours. The reaction mixture was quenched with saturated NaHCO₃ (200 mL), extracted with EtOAc (2×300 mL), and the organic layer was dried over Na₂SO₄, filtered and evaporated to afford a colourless residue. The crude product was purified by flash silica chromatography, elution gradient 0 to 50% THF in petroleum ether. Pure fractions were evaporated to dryness to afford (2S,3R,4S,5S,6S)-2-(2-(((tert-butoxycarbonyl)amino)methyl)-4-(hydroxymethyl)phenoxy)-6-(methoxycarbonyl)tetrahydro-2H-pyran-3,4,5-triyl triacetate (Intermediate 15, 18.80 g, 96%) as a pale yellow material. m/z (ES+), [M+Na]⁺=592; TFA, HPLC tR=1.088 min.

### Intermediate 16

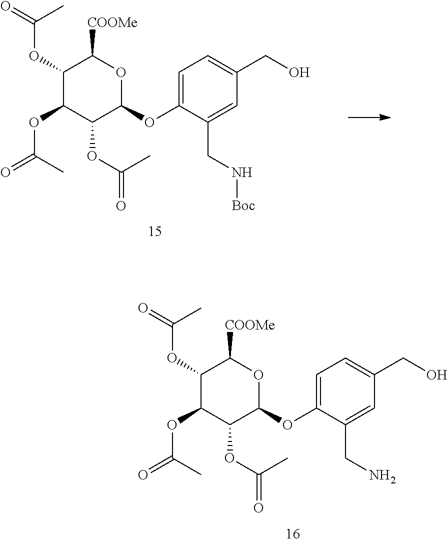 [SMILES: COC(=O)[C@H]1O[C@@H](Oc2ccc(CO)cc2CNC(=O)OC(C)(C)C)[C@H](OC(C)=O)[C@@H](OC(C)=O)[C@@H]1OC(C)=O ; COC(=O)[C@H]1O[C@@H](Oc2ccc(CO)cc2CN)[C@H](OC(C)=O)[C@@H](OC(C)=O)[C@@H]1OC(C)=O]

Intermediate 15 (10 g, 17.56 mmol) in HCl/1,4-dioxane (40 mL)/1,4-dioxane (40 mL) at 25° C. under nitrogen. The resulting mixture was stirred at 25° C. for 1 hour. The solvent was removed under reduced pressure to afford (2S,3R,4S,5S,6S)-2-(2-(aminomethyl)-4-(hydroxymethyl)phenoxy)-6-(methoxycarbonyl)tetrahydro-2H-pyran-3,4,5-triyl triacetate hydrochloride (Intermediate 16) as a white material. m/z (ES+), [M+H]+=470; TFA, HPLC tR=0.587 min.

### Intermediate 17

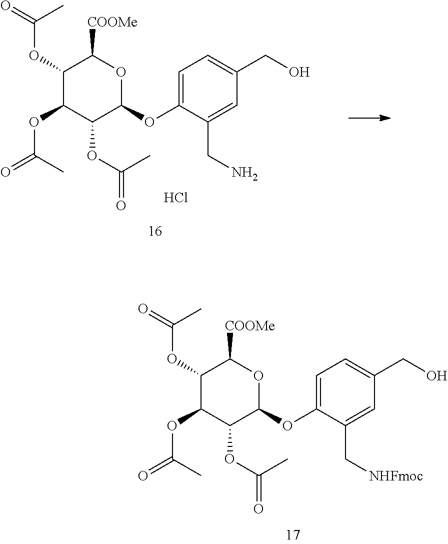

Intermediate 16 (1 g, 1.98 mmol), (9H-fluoren-9-yl)methyl carbonochloridate (0.767 g, 2.97 mmol) and DIPEA (1.036 mL, 5.93 mmol) in DCM (10 mL) was stirred at RT for 3 hours. The reaction mixture was quenched with water (50 mL), extracted with DCM (2×50 mL), the combined organic layer was dried over Na₂SO₄, filtered and evaporated to afford a residue. The crude product was purified by flash silica chromatography, elution gradient 0 to 80% petroleum ether in EtOAc. Pure fractions were evaporated to dryness to afford (2S,3R,4S,5S,6S)-2-(2-(((((9H-fluoren-9-yl)methoxy)carbonyl)amino)methyl)-4-(hydroxymethyl)phenoxy)-6-(methoxycarbonyl)tetrahydro-2H-pyran-3,4,5-triyl triacetate (Intermediate 17, 0.952 g, 69.6%) as a white material. m/z (ES+), [M+H]+=692; TFA, HPLC tR=0.880 min.

### Intermediate 18

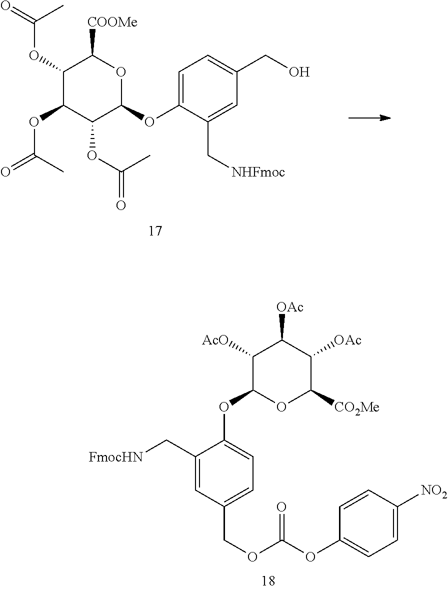

Intermediate 17 (3.5 g, 5.06 mmol), bis(4-nitrophenyl) carbonate (1.693 g, 5.57 mmol) and DIEA (1.326 mL, 7.59 mmol) in DMF (35 mL) was stirred at RT for 3 hours. The reaction mixture was quenched with water (50 mL), extracted with EtOAc (2×50 mL), and the combined organic layer was dried over Na₂SO₄, filtered and evaporated to afford residue. The crude product was purified by flash silica chromatography, elution gradient 0 to 100% EtOAc in petroleum ether. Pure fractions were evaporated to dryness to afford (2S,3R,4S,5S,6S)-2-(2-(((((9H-fluoren-9-yl)methoxy)carbonyl)amino)methyl)-4-((((4-nitrophenoxy)carbonyl)oxy)methyl)phenoxy)-6-(methoxycarbonyl)tetrahydro-2H-pyran-3,4,5-triyl triacetate (Intermediate 18, 3.68 g, 85%) as a white material. m/z (ES+), [M+H]+=857; fa, HPLC tR=1.007 min.

### Intermediate 19

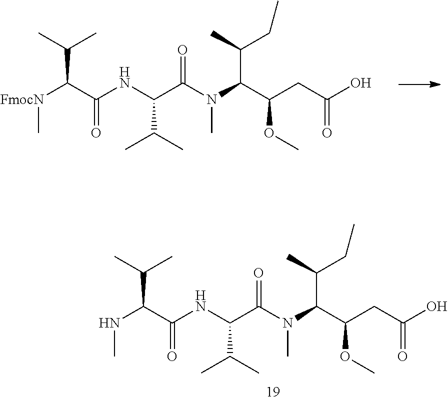

(5S,8S,11S,12R)-11-((S)-sec-butyl)-1-(9H-fluoren-9-yl)-5,8-diisopropyl-12-methoxy-4,10-dimethyl-3,6,9-trioxo-2-oxa-4,7,10-triazatetradecan-14-oic acid (12 g, 18.81 mmol) in trimethylamine (80 mL) was stirred at 70° C. for 3 hours. The solvent was removed under reduced pressure. The precipitate was collected by filtration, washed with petroleum ether (100 mL) and dried under vacuum to afford (3R,4S,5S)-4-((S)—N,3-dimethyl-2-((S)-3-methyl-2-(methylamino)butanamido)butanamido)-3-methoxy-5-methylheptanoic acid (Intermediate 19, 5.80 g, 74.2%) as a white solid. LCMS (3 mins): No PDA; 416 [M+H]+.

### Intermediate 20

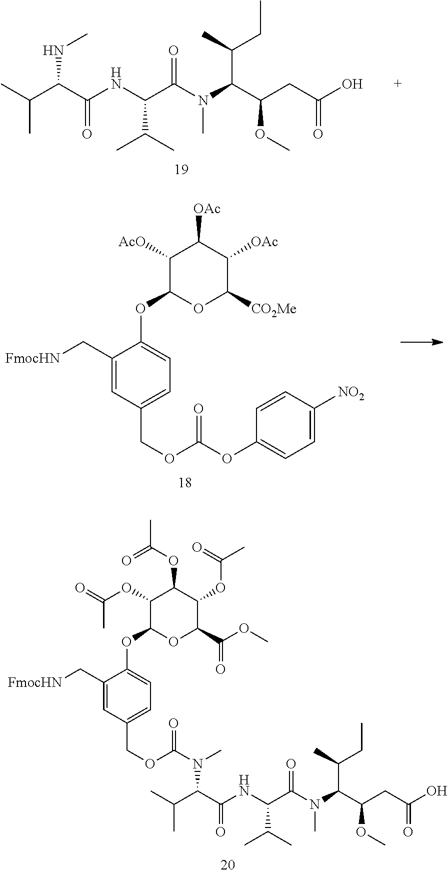

Intermediate 18 (1.5 g, 1.75 mmol), Intermediate 19 (0.728 g, 1.75 mmol), DIPEA (0.917 mL, 5.25 mmol) and 2-Hydroxypyridine-N-oxide (0.195 g, 1.75 mmol) in DMA (200 mL) were stirred at RT for 18 hours. The reaction mixture was extracted with ethyl acetate (3×50 mL), the organic layer was dried over Na₂SO₄, filtered and evaporated to afford yellow material. The crude product was purified by preparative HPLC (Column: XBRIDGE Prepl phenyl OBD Colum, 30*150 nm 5 um; Mobile Phase A: Water(10 mmol/L NH₄HCO₃), Mobile Phase B: ACN; Flow rate: 60 mL/min; Gradient: isocratic 54-59; Wave Length: 220/254 nm; RT1(min): 15. Fractions containing the desired compound were evaporated to dryness to afford (5S,8S,11S,12R)-1-(3-(((((9H-fluoren-9-yl)methoxy)carbonyl)amino)methyl)-4-(((2S,3R,4S,5S,6S)-3,4,5-triacetoxy-6-(methoxycarbonyl)tetrahydro-2H-pyran-2-yl)oxy)phenyl)-11-((S)-sec-butyl)-5,8-diisopropyl-12-methoxy-4,10-dimethyl-3,6,9-trioxo-2-oxa-4,7,10-triazatetradecan-14-oic acid (Intermediate 20, 1.400 g, 70.6%) as a material. m/z (ES+), [M+H]+=1134; FA, HPLC tR=1.197 min.

### Intermediate 21

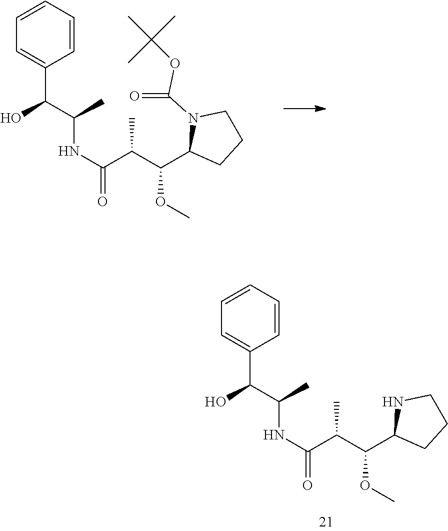 [SMILES: CO[C@H]([C@@H](C)C(=O)N[C@H](C)[C@@H](O)c1ccccc1)[C@@H]1CCCN1C(=O)OC(C)(C)C ; CO[C@@H]([C@@H]1CCCN1)[C@@H](C)C(=O)N[C@H](C)[C@@H](O)c1ccccc1]

To tert-butyl (S)-2-((1R,2R)-3-(((1S,2R)-1-hydroxy-1-phenylpropan-2-yl)amino)-1-methoxy-2-methyl-3-oxopropyl)pyrrolidine-1-carboxylate (256 mg, 0.61 mmol) in DCM (2 mL) was added TFA (2 mL). The reaction mixture was stirred at room temperature for 2 h. The reaction mixture was concentrated under reduced pressure to give a crude (2R,3R)—N-((1S,2R)-1-hydroxy-1-phenylpropan-2-yl)-3-methoxy-2-methyl-3-((S)-pyrrolidin-2-yl)propanamide (Intermediate 21) which was used without purification. LCMS (3 min): 0.55 min; 322.0 [M+H]+

### Intermediate 22

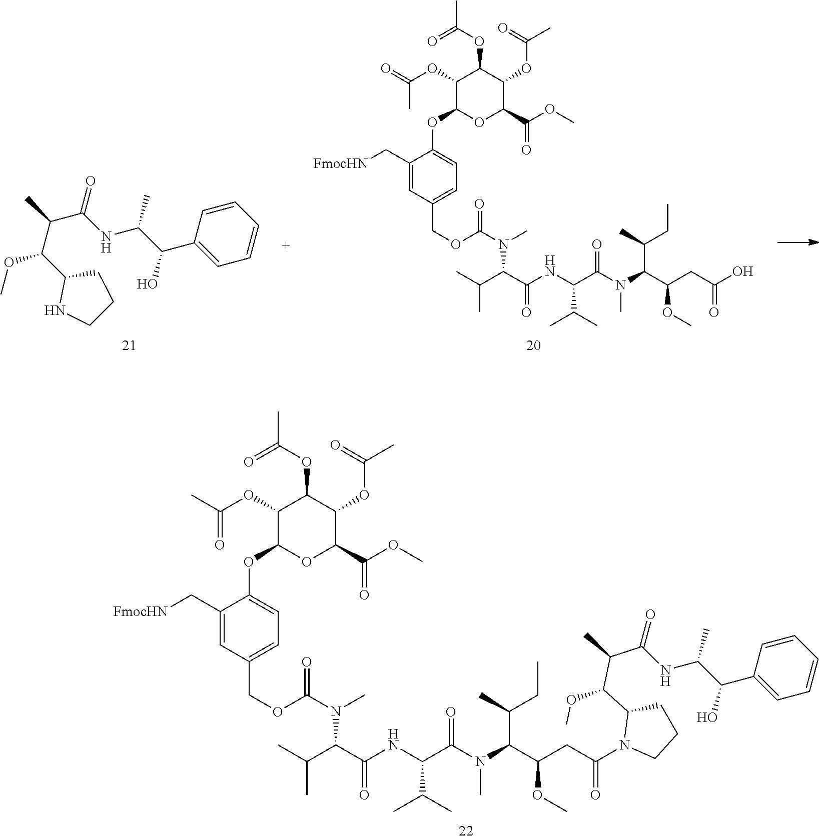

To crude Intermediate 21 (234 mg, 0.73 mmol) in DMF (5 mL) was added Intermediate 20 (486 mg, 0.429 mmol), HATU (269 mg, 0.71 mmol) and DIPEA (0.360 mL, 2.07 mmol). The reaction mixture was stirred at room temperature for 30 mins. The reaction mixture was concentrated under reduced pressure to give a crude. Purification by column chromatography (0-6% MeOH in DCM) gave (2S,3R,4S,5S,6S)-2-(2-(((((9H-fluoren-9-yl)methoxy)carbonyl)amino)methyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (Intermediate 22, 552 mg, 90%) as a colourless material. LCMS (15 min): 8.99 min; 1436.9 [M+H]+

### Intermediate 23

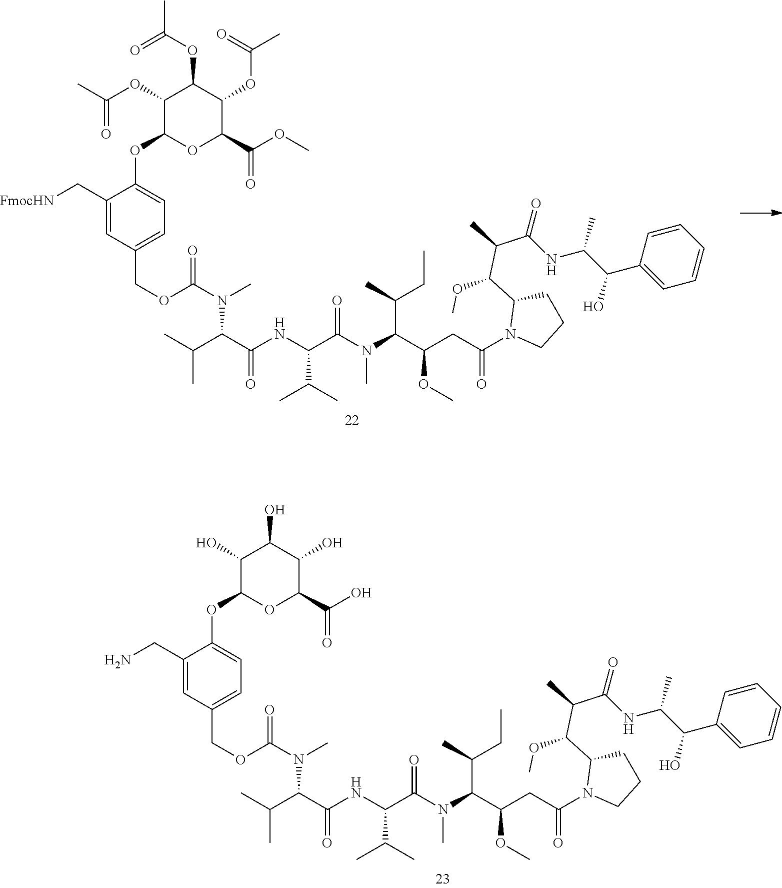

To Intermediate 22 (552 mg, 0.38 mmol) in MeOH (8 mL) and water (2 mL) was added potassium carbonate (531 mg, 3.84 mmol). The reaction mixture was stirred at room temperature for 3 h. Acetic acid (1.1 mL) was added and the reaction mixture purified by reverse phase chromatography (C18, 120 g, 5-40% MeCN in water+0.1% FA, 25 CV) to give (2S,3S,4S,5R,6S)-6-(2-(aminomethyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (Intermediate 23, 259 mg, 62.8%) as a white material. LCMS (15 min): 5.57 min; 1074.9 [M+H]⁺.

### Synthesis of LP2

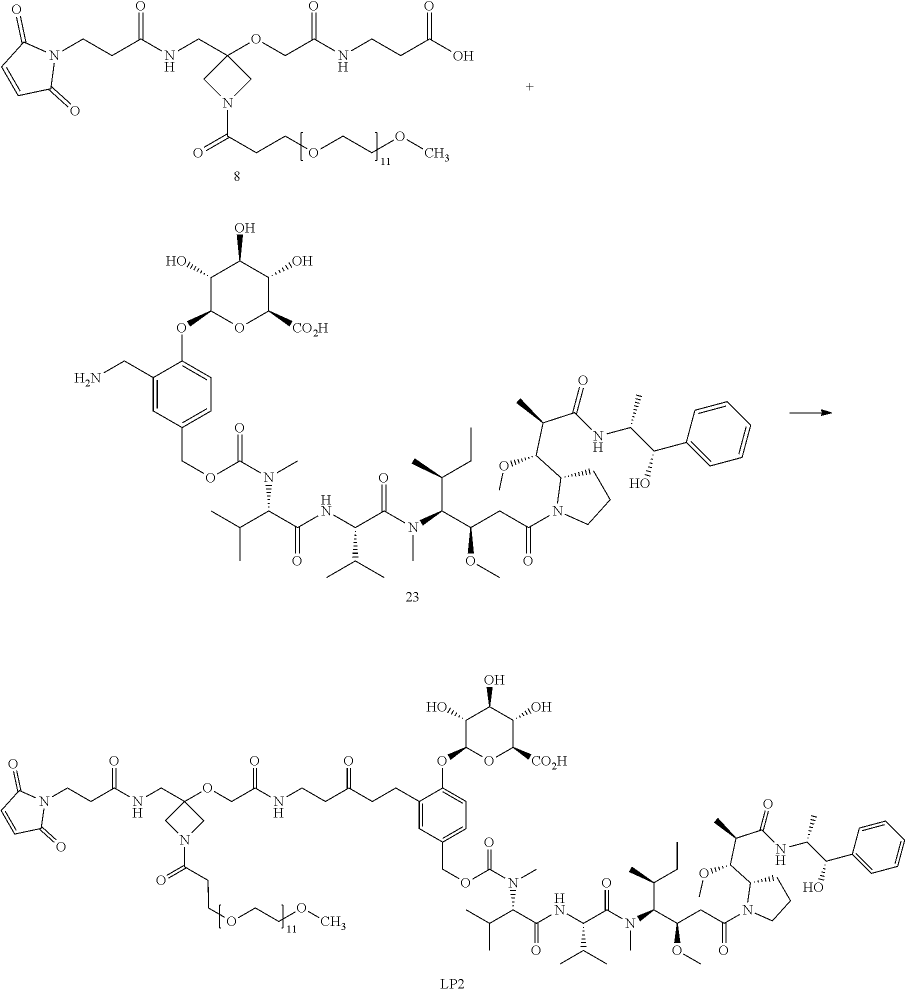

To the solution of Intermediate 8 (460 mg, 0.5 mmol) in DMF (4.6 ml) was added the solution of Intermediate 23 (520 mg, 0.48 mmol), HBTU (275 mg, 0.49 mmol) and DIEA (187 mg, 0.48 mmol) at −15 to −10° C. After stirring for 10 mins, the crude residue was quenched with HCl in 1,4-dioxane. The crude product was purified by prep-HPLC (column: C18 reverse column; gradient: MeCN in water (0.1 A % FA): 25-40%)) to afford (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((3-(2-((3-((3-(2,5-dioxo-2,5-dihydro-1H-pyrrol-1-yl)propanamido)methyl)-1-(2,5,8,11,14,17,20,23,26,29,32,35-dodecaoxaoctatriacontan-38-oyl)azetidin-3-yl)oxy)acetamido)propanamido)methyl)phenoxy)-3,4,5-trihydroxytetrahydro-2H-pyran-2-carboxylic acid as a light yellow solid. (LP2, 190 mg, yield: 20%). MS ESI: [M/2+H]+m/z 1004.4.

### Intermediate 24

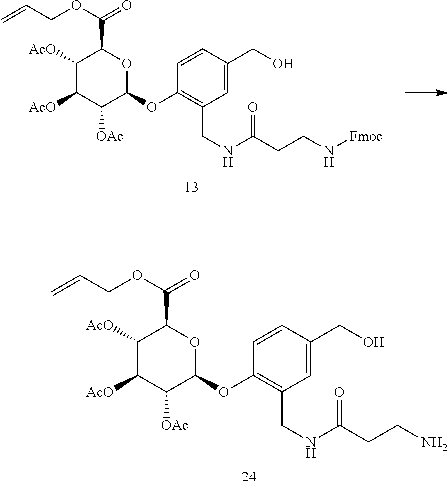

To a stirred reactor containing Intermediate 13 (1 kg, 1.27 mol) and tetrahydrofuran (10 L) at −45° C. under nitrogen was added 2,3,4,6,7,8,9,10-octahydropyrimido[1,2-a]azepine (385.98 g, 2.54 mol). The mixture was stirred at −45° C. for four hours then diluted with acetonitrile (5 L) and quenched by the addition of hydrogen chloride in tert-butyl methyl ether solution (2.54 L, 2.0 M, 5.07 mol). The mixture was concentrated to ~5 L under vacuum, and diluted with n-heptane (5 L). The acetonitrile layer was collected containing (2S,3S,4S,5R,6S)-2-((allyloxy)carbonyl)-6-(2-((3-aminopropanamido)methyl)-4-(hydroxymethyl)phenoxy)tetrahydro-2H-pyran-3,4,5-triyl triacetate (Intermediate 24, 3.88 kg of MeCN solution, 89.97% area, assumed 100%). LCMS m/z 566.6 [M+H]⁺.

### Alternative Synthesis of LP2

### General Information for Alternative Synthesis of LP2

Flash chromatography was performed using a BIOTAGE ISOLERA and fractions checked for purity using thin-layer chromatography (TLC). TLC was performed using MERCK KIESELGEL 60 F254 silica gel, with fluorescent indicator on aluminium plates. Visualisation of TLC was achieved with UV light.

Extraction and chromatography solvents were bought and used without further purification from VWR U.K.

All fine chemicals were purchased from SIGMA-ALDRICH unless otherwise stated.

Pegylated reagents were obtained from QUANTA BIODESIGN US via STRATECH UK.

LC/MS conditions for Alternative Synthesis of LP2 Positive mode electrospray mass spectrometry was performed using a WATERS ACQUITY H-CLASS SQD2 using one of the following methods.

The HPLC (WATERS ALLIANCE 2695) was run using a mobile phase of water (A) (TFA 0.03%) and acetonitrile (B) (TFA 0.03%).

LCMS 16.0 min: Initial composition 5% B increased to 25% B over a 3 minute period. The composition was then increased to 30% B over a 5 minute period, followed by increasing to 95% B over a 4 minute period. The composition was held for a minute at 95% B, then returned to 5% B in 10 seconds and held there for 2 minutes 50 seconds. The total duration of the gradient run was 16.0 minutes. Flow rate was 0.40 mL/minute. Wavelength detection range: 210 nm. Columns: WATERS ACQUITY UPLC BEH C18 1.7 um, 2.1*100 mm at 40° C.

LCMS 16.5 min: Initial composition 10% B held over 25 seconds, then increased from 5% B to 90% B over a 11 minute 35 seconds' period. The composition was held for 70 seconds at 90% B, then returned to 5% B in 10 seconds and held there for 3 minutes. The total duration of the gradient run was 16.5 minutes. Flow rate was 0.40 mL/minute. Wavelength detection range: 210 to 220 nm. Columns: WATERS ACQUITY UPLC BEH C18 1.7 um, 2.1*100 mm at 40° C.

LCMS 26.0 min: Initial composition 10% B increased to 30% B over a 5 minute period, followed by increasing to 35% B over a 15 minute period. The composition was increased to 90% B over a 2 minute period, held for 1 minute at 90% B, then returned to 10% B in 10 seconds and held there for 2 minutes and 50 seconds. The total duration of the gradient run was 26.0 minutes. Flow rate was 0.40 mL/minute. Wavelength detection range: 210 nm. Columns: WATERS ACQUITY UPLC BEH C18 1.7 um, 2.1*100 mm at 40° C.

LCMS 30.0 min: Initial composition 10% B increased from 45% B over a 5 minute period. The composition was increased to 47% B over 15 minutes, then increased to 95% B over 5 minutes. The composition was held at 95% B for 2 minutes, then returned to 10% B in 10 seconds and held there for 2 minutes 50 seconds. The total duration of the gradient run was 30 minutes. Flow rate was 0.40 mL/minute. Wavelength detection range: 210 nm. Columns: WATERS ACQUITY UPLC BEH C18 1.7 um, 2.1*100 mm at 55° C.

LCMS 31.0 min: Initial composition 10% B increased from 45% B over a 5 minute period. The composition was increased to 50% B over 20 minutes, then increased to 90% B over 2 minutes. The composition was held at 90% B for 1 minute, then returned to 10% B in 10 seconds and held there for 2 minutes 50 seconds. The total duration of the gradient run was 31 minutes. Flow rate was 0.40 mL/minute. Wavelength detection range: 210 nm. Columns: WATERS ACQUITY UPLC BEH C18 1.7 um, 2.1*100 mm at 40° C.

LCMS 31.0 min: Initial composition 10% B increased from 45% B over a 20 minute period. The composition was increased to 95% B over 5 minutes, held at 95% B for 2 minute, then decreased to 5% B in 10 seconds and held there for 2 minutes 50 seconds. The total duration of the gradient run was 31 minutes. Flow rate was 0.40 mL/minute. Wavelength detection range: 210 nm. Columns: WATERS ACQUITY UPLC CSH Phenyl-Hexyl 1.7 um, 2.1*100 mm at 40° C.

### NMR Method for Alternative Synthesis of LP2

Proton NMR chemical shift values were measured on the delta scale at 300 MHz or 400 MHz using a BRUKER AV400. The following abbreviations have been used: s, singlet; d, doublet; t, triplet; q, quartet; quin, quintet; m, multiplet; br, broad. Coupling constants are reported in Hz.

### Intermediate A

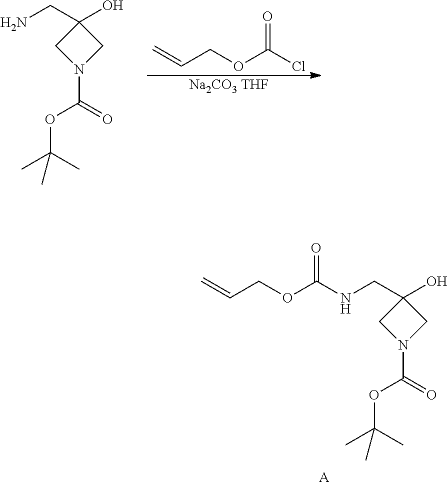

To a stirred reactor containing tert-butyl 3-(aminomethyl)-3-hydroxy-azetidine-1-carboxylate (290 g, 1.0 eq, 1.48 mol) and allyl chloroformate (214 g, 1.2 eq, 1.78 mmol) in tetrahydrofuran (2.90 L) was added sodium carbonate (234 g, 1.5 eq, 2.22 mol). The contents of the reactor was stirred at 25° C. for 2 hours. Water (2.90 L) was added to the reactor, and the resulting mixture was extracted twice with ethylacetate (2.90 L×2). The combined organic layer was washed with brine (2.90 L) and then concentrated under reduced pressure down to a volume of 15 mL. MTBE (25 mL) was added and then the reaction was concentrated to 15 mL. Further MTBE (1.45 L) was added and the reaction was again concentrated down to 15 mL. MTBE (1.45 L) was added and the mixture stirred for 4 hours at 25° C. The solid formed was collected by filtration, washed with MTBE (580 mL) and dried under vacuum at 45° C. to give tert-butyl 3-[(allyloxycarbonylamino)methyl]-3-hydroxy-azetidine-1-carboxylate, Intermediate A as a solid (301 g, 98.9 w/w %, 70.1% yield); ¹H NMR (500 MHz, DMSO-d6) δ ppm 1.37 (s, 9H) 3.15 (d, J=6.10 Hz, 2H) 3.50-3.65 (m, 2H) 3.81 (br d, J=8.54 Hz, 2H) 4.48 (br d, J=5.19 Hz, 2H) 5.16 (dd, J=10.38, 1.53 Hz, 1H) 5.27 (dd, J=17.24, 1.68 Hz, 1H) 5.75 (s, 1H) 5.90 (ddt, J=17.17, 10.60, 5.19, 5.19 Hz, 1H) 7.40 (t, J=6.26 Hz, 1H) LCMS (ESI) m/z 287 (M+H)+.

### Intermediate B

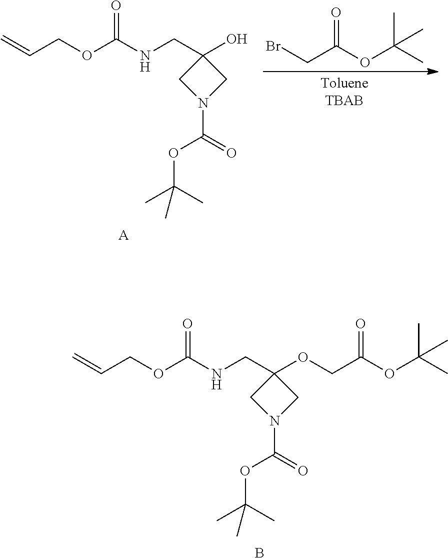

To a stirred reactor containing Intermediate A (100.0 g, 0.35 mol, 1.0 eq) added TBAB (20.0 g, 0.06 mol, 20 w/w %), and NaOH (15.37 g, 0.38 mol, 1.1 eq) in toluene (1 L, 10 V) at 5° C. was added tert-butyl bromoacetate (81.7 g, 0.42 mol, 1.2 eq.) dropwise. The reaction mixture was stirred at 25° C. for 16 hours. Water (500 mL, 5 V) was added to the reactor and stirred for 5 min The layers were allowed to separate and the aqueous layer discarded. The organic layer was washed with brine twice (2×5 V, 10%) and then concentrated to dryness. MTBE (200 mL, 2 V) was added to the residue and stirred. N-heptane (300 mL, 3V) was added and the reaction mixture was cooled to 5° C. and stirred for 6 h. The resulting solid was filtered, washed with MTBE:heptane (80 mL:120 mL, 2:3, 2 V) and dried to a constant weight under vacuum at 45° C. to give tert-butyl 3-[(allyloxycarbonylamino)methyl]-3-(2-tert-butoxy-2-oxo-ethoxy)azetidine-1-carboxylate, Intermediate B as a solid (100 g, 98.3 w/w %, 71.0% yield); ¹H NMR (500 MHz, CHLOROFORM-d) δ ppm 1.44 (s, 9H) 1.47-1.50 (m, 9H) 3.51 (br d, J=4.88 Hz, 2H) 3.78 (br d, J=9.46 Hz, 2H) 3.85 (d, J=9.77 Hz, 2H) 3.98 (s, 2H) 4.58 (br d, J=5.49 Hz, 2H) 5.22 (br d, J=10.38 Hz, 1H) 5.31 (dd, J=17.09, 1.53 Hz, 1H) 5.74 (br s, 1H) 5.93 (ddt, J=16.86, 10.91, 5.49, 5.49 Hz, 1H); LCMS (ESI) m/z 401 (M+H)⁺.

### Intermediate D

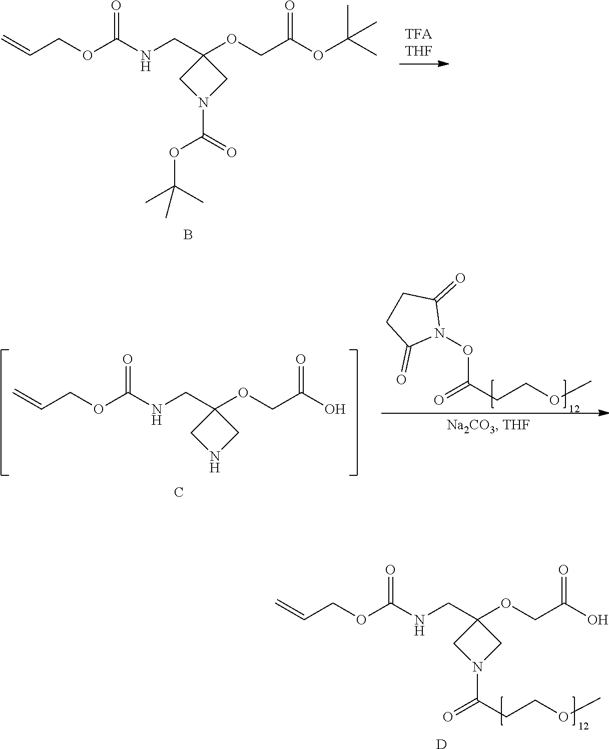

To a stirred solution of Intermediate B (211.3 g, 0.53 mol, 1.0 eq) in THF (1.69 L, 8 V) was added TFA (1.69 L, 8.0 V). The reaction mixture was stirred at 50° C. for 24 h. The reaction mixture was concentrated to dryness to give 2-[3-[(allyloxycarbonylamino)methyl]azetidin-3-yl]oxyacetic acid (215.1 g, 54.1 w/w %, 90.3% yield) which was used directly in the next step without purification. To a stirred solution of 2-[3-[(allyloxycarbonylamino)methyl]azetidin-3-yl]oxyacetic acid (203.3 g, 54.1 w/w %) in THF (1.1 L, 10 V) at 5° C. was added MPEG11-NHS (308.9 g, 0.45 mol, 1.0 eq) and Na₂CO₃ (143.2 g, 1.35 mol, 3.0 eq). The reaction mixture was stirred for 2 h at 25° C. The solvent was removed under vacuum and water (1.1 L, 10 V) and MTBE (1.1 L, 10 V) were added to the residue. The organic layer was separated and discarded. The aqueous layer was washed twice with MTBE (1.1 L×2, 10 V×2); Note product in the aqueous phase. The pH of the aqueous phase was adjusted to 3-4 by the addition of HCl (1 M, 66 mL, ~0.6 V) The aqueous phase was extracted four times with EtOAc (1.1 L×4, 10 V×4); Note product now in organic phase. The combined organic layers were concentrated to dryness under vacuum to give 2-[3-[(allyloxycarbonylamino)methyl]-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetic acid, Intermediate D as an oil (349.1 g, 80.0 w/w %, 76.1% yield); ¹H NMR (500 MHz, CHLOROFORM-d) δ ppm 2.29-2.41 (m, 2H) 3.34 (s, 3H) 3.41 (s, 1H) 3.41-3.63 (m, 44H) 3.64-3.77 (m, 4H) 3.82 (br d, J=10.99 Hz, 1H) 3.92 (br d, J=10.99 Hz, 1H) 4.01-4.17 (m, 4H) 4.52 (br d, J=5.49 Hz, 2H) 5.17 (br d, J=10.38 Hz, 1H) 5.22-5.29 (m, 1H) 5.87 (ddt, J=16.78, 10.91, 5.53, 5.53 Hz, 1H) 6.05 (br t, J=5.80 Hz, 1H); LCMS (ESI) m/z 815.1 (M+H)+.

### Intermediate E

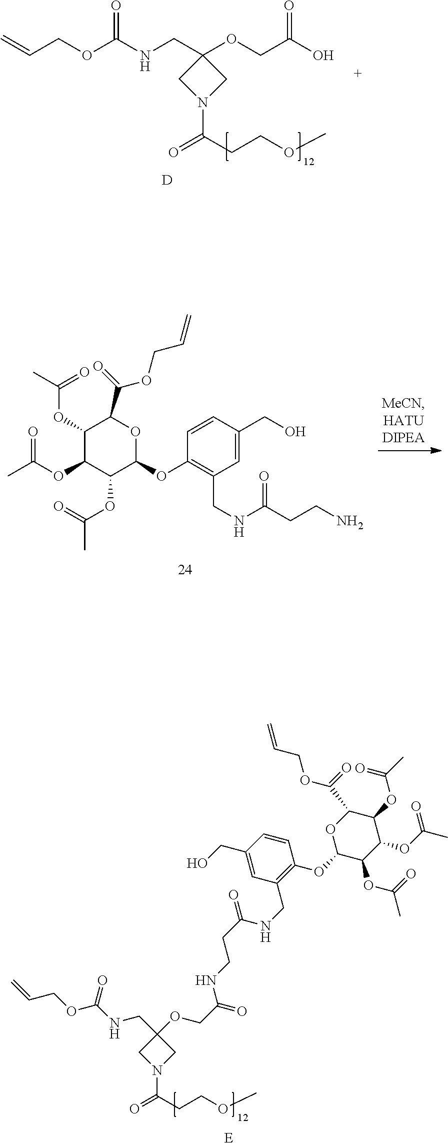

To a stirred solution of Intermediate D (62.5 g, 80.0 w/w %, 0.06 mol, 1.0 eq) and Intermediate 24 (566.6 g, 0.06 mol, 1.05 eq) in MeCN (500 mL, 10 V) at 0° C. was added HATU (34.2 g, 0.09 mol, 1.5 eq) and DIPEA (23.3 g, 0.18 mol, 3.0 eq). The reaction mixture was stirred at 0° C. for 30 min, then water (500 mL, 10 V) was charged. The layers were separated and both retained. The aqueous phase was extracted twice with DCM (500 mL×2, 10 V×2). The combined organic layers were washed twice with brine (500 mL×2, 10 V×2) and then concentrated to dryness under vacuum at 30-40° C. to give allyl (2S,3S,4S,5R,6S)-3,4,5-triacetoxy-6-[2-[[3-[[2-[3-[(allyloxycarbonylamino)methyl]-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-(hydroxymethyl)phenoxy]tetrahydropyran-2-carboxylate, Intermediate E as an oil (56.3 g, 81.2 w/w %, 54.6% yield); ¹H NMR (500 MHz, DMSO-d6) 1.21-1.31 (m, 1H) 1.99 (d, J=7.32 Hz, 6H) 2.05 (s, 3H) 2.29 (t, J=6.41 Hz, 2H) 2.35-2.44 (m, 2H) 3.24 (s, 3H) 3.27-3.49 (m, 41H) 3.56-3.66 (m, 2H) 3.71-3.81 (m, 2H) 3.87-4.00 (m, 3H) 4.05-4.12 (m, 2H) 4.18-4.25 (m, 1H) 4.41 (d, J=4.88 Hz, 2H) 4.47-4.56 (m, 3H) 4.60-4.68 (m, 1H) 4.76 (d, J=10.07 Hz, 1H) 5.08-5.19 (m, 4H) 5.23-5.35 (m, 3H) 5.48 (t, J=9.61 Hz, 1H) 5.56 (d, J=7.93 Hz, 1H) 5.84-5.95 (m, 2H) 7.01 (d, J=8.54 Hz, 1H) 7.12 (s, 1H) 7.17 (d, J=8.07 Hz, 1H) 7.51 (br t, J=6.10 Hz, 1H) 7.91 (br t, J=5.65 Hz, 1H) 8.24 (t, J=5.95 Hz, 1H); LCMS (ESI) m/z 682.1 (M+2H)²⁺.

### Intermediate F

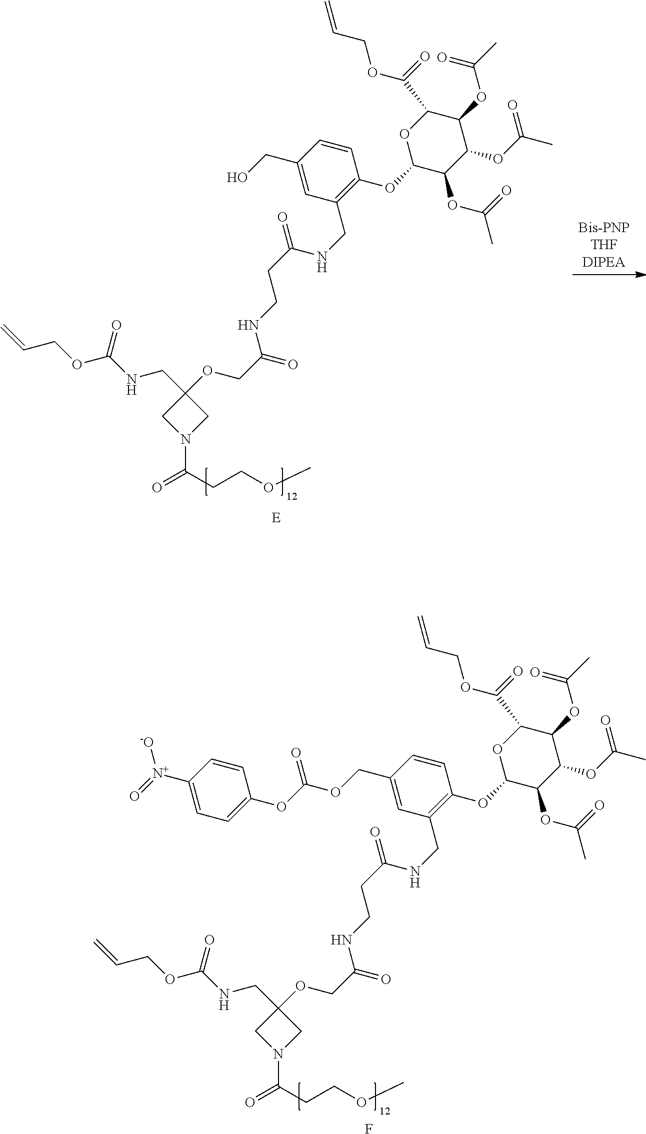

To a stirred solution of Intermediate E (40 g, 81.2 w/w %, 24 mmol, 1.0 eq) in THF (488 mL, 15 V) at 25° C. was added Bis-PNP (25.6 g, 84 mmol, 3.5 eq) and DIPEA (7.1 g, 55 mmol, 2.3 eq)). The reaction mixture was stirred at 25° C. for 16 h. The reaction was cooled to 10° C. then NaCl (15% aq; 325 mL, 10 V) was charged. The pH of the reaction mixture was adjusted to pH 3-6 by the addition of HCl (1M, 19.5 mL, 0.6 V) at 10° C. The layers were allowed to separate and the aqueous layer discarded. The organic layer was concentrated to 10 V and then MTBE (1.63 L, 50 V) was added dropwise. The resulting oil was collected to give allyl ((2S,3S,4S,5R,6S)-3,4,5-triacetoxy-6-[2-[[3-[[2-[3-[(allyloxycarbonylamino)methyl]-1-[3-[2-[2-[2-[2-[2-[2-[2-[2-[2-[2-(2-methoxyethoxy) ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-[(4-nitrophenoxy)carbonyloxymethyl]phenoxy]tetrahydropyran-2-carboxylate, Intermediate F, as an oil (40.8 g, 76.0 w/w %, 85.2% yield; ¹H NMR (300 MHz, DMSO-d6) δ ppm) 1.96-2.09 (m, 9H) 2.21-2.35 (m, 2H) 2.36-2.47 (m, 2H) 3.24 (s, 3H) 3.25-3.50 (m, 43H) 3.55-4.00 (m, 8H) 4.05-4.15 (m, 2H) 4.16-4.29 (m, 1H) 4.44-4.67 (m, 4H) 4.80 (d, J=9.96 Hz, 1H) 5.08-5.37 (m, 8H) 5.44-5.57 (m, 1H) 5.65 (d, J=7.81 Hz, 1H) 5.80-5.98 (m, 2H) 7.11 (d, J=8.44 Hz, 1H) 7.28 (s, 1H) 7.37 (br d, J=8.44 Hz, 1H) 7.48-7.61 (m, 3H) 7.92 (br t, J=5.56 Hz, 1H) 8.26-8.37 (m, 3H)); LCMS (ESI) m/z 764.6 (M+2H)²⁺.

### Intermediate G

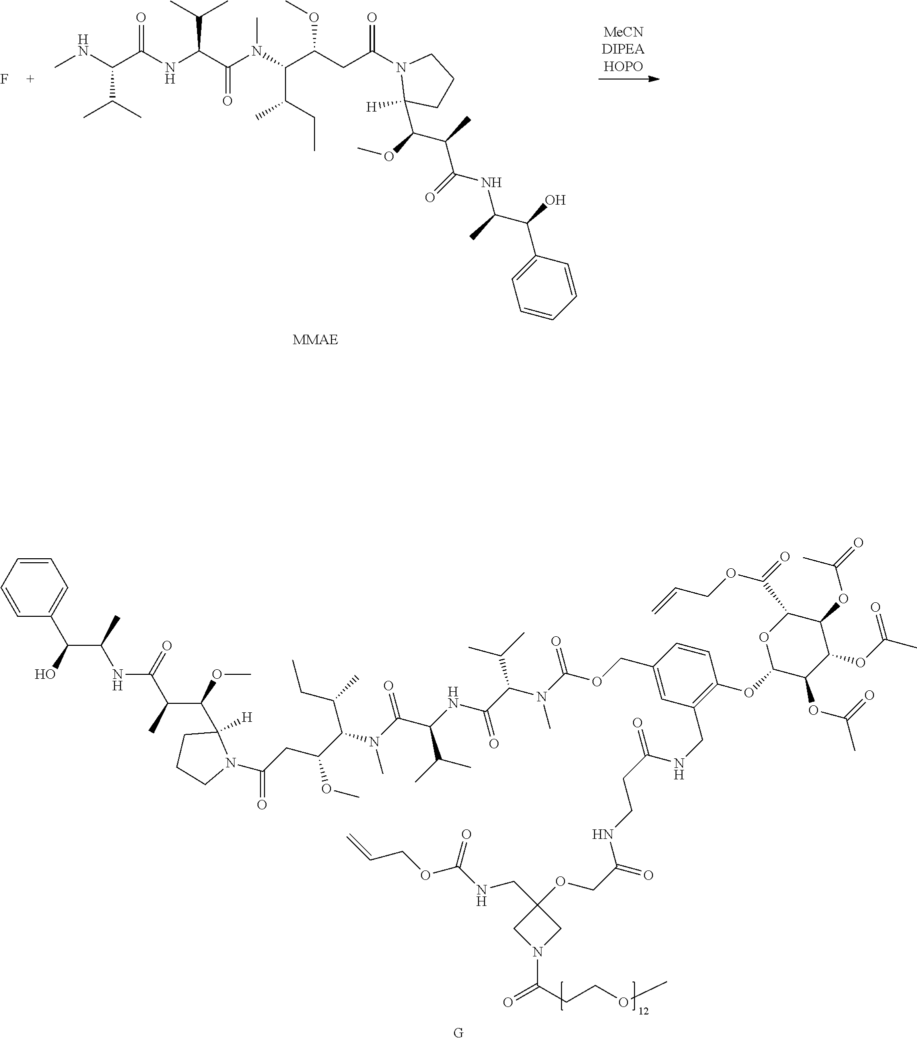

To a stirred mixture of Intermediate F (39.5 g, 76.0 w/w %, 20 mmol, 1.0 eq), and MMAE (12.8 g, 24 mmol, 1.2 eq) in MeCN (20 V) at 25° C. was added DIPEA (4.4 g, 34 mmol, 1.7 eq) dropwise, followed by HOPO (3.8 g, 34 mmol, 1.7 eq). The reaction mixture was stirred for 7 h at 25° C. The reaction was cooled to 10° C. then NaCl (15% aq, 300 mL, 10 V) was charged. The pH of the reaction mixture was adjusted to pH 3-6 by the addition of HCl (1M, 9 mL, 0.6 V) at 10° C. The layers were allowed to separate and the aqueous layer discarded. The organic layer was concentrated to 3-5 V to give allyl (2S,3S,4S,5R,6S)-3,4,5-triacetoxy-6-[2-[[3-[[2-[3-[(allyloxycarbonylamino)methyl]-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenoxy]tetrahydropyran-2-carboxylate, Intermediate G, as an acetonitrile solution (112.3 g, 31.6 w/w %, 85.8% yield). A sample was concentrated to dryness to give a white solid for characterisation; ¹H NMR (500 MHz, DMSO-d6) δ ppm 0.72-0.94 (m, 18H) 0.95-1.07 (m, 6H) 1.30 (br s, 1H) 1.44-1.60 (m, 2H) 1.67-1.85 (m, 3H) 1.95-2.02 (m, 6H) 2.04 (s, 3H) 2.05-2.14 (m, 2H) 2.29 (br t, J=6.26 Hz, 3H) 2.40 (br s, 3H) 2.80-2.91 (m, 3H) 2.97 (br s, 1H) 3.00-3.09 (m, 1H) 3.12 (br s, 2H) 3.16-3.26 (m, 10H) 3.28-3.36 (m, 3H) 3.41-3.43 (m, 3H) 3.50 (s, 43H) 3.53-3.65 (m, 5H) 3.71-3.82 (m, 3H) 3.90-4.11 (m, 8H) 4.16-4.30 (m, 2H) 4.35-4.46 (m, 1H) 4.46-4.54 (m, 4H) 4.61 (br d, J=13.73 Hz, 2H) 4.78 (br d, J=10.07 Hz, 2H) 4.94-5.12 (m, 3H) 5.15-5.19 (m, 2H) 5.21-5.37 (m, 4H) 5.41-5.53 (m, 2H) 5.60 (br d, J=5.49 Hz, 1H) 5.84-5.94 (m, 2H) 7.04 (br d, J=5.19 Hz, 1H) 7.13-7.23 (m, 3H) 7.24-7.28 (m, 2H) 7.29-7.32 (m, 2H) 7.53 (br t, J=5.95 Hz, 1H) 7.93 (br s, 2H) 8.28 (br s, 1H); LCMS (ESI) m/z 1054.1 (M+2H)²⁺.

### Intermediate H

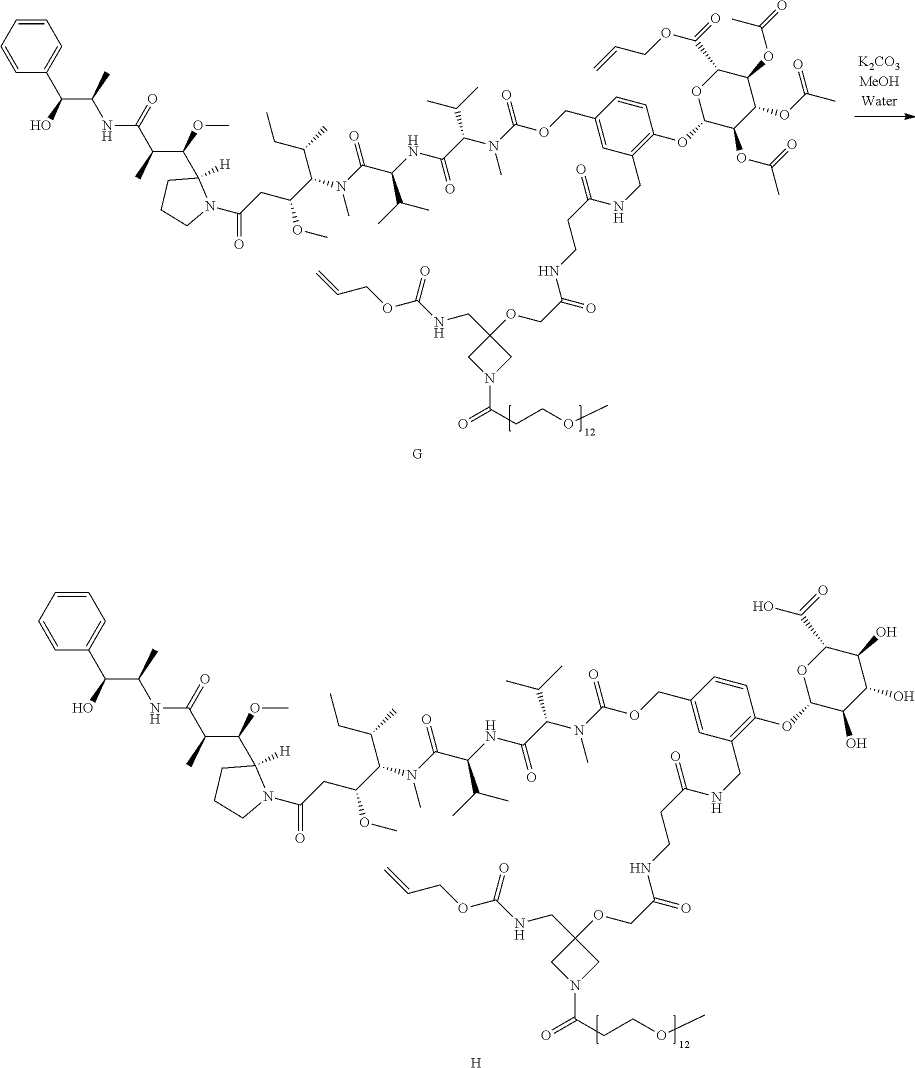

A stirred solution of Intermediate G (99 g g, 31.6 w/win MCN, 14.9 mmol, 1.1 eq) was concentrated to dryness. MeOH (156 mL, 5 V) was added and the mixture was again concentrated to dryness. The residue was dissolved in MeOH (313 mL, 10 V) at 5° C. and to this solution was added a solution of K₂CO₃ (313 mL, 0.35M aq, 7.5 eq). The solution was stirred for 10 h at 23° C., then cooled to 0° C. Citric acid (2000 aq, 250.4 mL, 8V) was added to adjust the pH to 3-5. The reaction mixture was washed twice with iPrOAc (2×313 mL; 2×10 V); Note product was in the aqueous layer. The aqueous phase was purified by reverse phase HPLC to give (2S,3S,4S,5R,6S)-6-[2-[[3-[[2-[3-[(allyloxycarbonylamino)methyl]-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-[[[(1S)-1-[[(1S)-1-[[(1S,2R)-4-[(2S)-2-[(1R,2R)-3-[[(1R,2S)-2-hydroxy-1-methyl-2-phenyl-ethyl]amino]-1-methoxy-2-methyl-3-oxo-propyl]pyrrolidin-1-yl]-2-methoxy-1-[(1S)-1-methylpropyl]-4-oxo-butyl]-methyl-carbamoyl]-2-methyl-propyl]carbamoyl]-2-methyl-propyl]-methyl-carbamoyl]oxymethyl]phenoxy]-3,4,5-trihydroxy-tetrahydropyran-2-carboxylic acid, Intermediate H, as a solid (30.1 g, 77.6 w/w %, 81.0% yield); ¹H NMR (500 MHz, DMSO-d6) δ ppm 0.73-0.94 (m, 17H) 0.95-1.06 (m, 6H) 1.30 (br s, 1H) 1.44-1.60 (m, 2H) 1.67-1.85 (m, 3H) 1.92-2.15 (m, 3H) 2.23-2.45 (m, 6H) 2.77-2.93 (m, 3H) 2.97 (br s, 1H) 3.00-3.09 (m, 1H) 3.12 (br s, 2H) 3.16-3.48 (m, 30H) 3.55-3.60 (m, 3H) 3.69-3.80 (m, 2H) 3.84-4.05 (m, 5H) 4.11 (br d, J=9.16 Hz, 1H) 4.15-4.29 (m, 2H) 4.33-4.51 (m, 4H) 4.57 (br s, 1H) 4.97-5.18 (m, 3H) 5.27 (dd, J=17.09, 1.53 Hz, 1H) 5.45 (br s, 1H) 5.85-5.94 (m, 1H) 7.06 (br dd, J=8.24, 3.97 Hz, 2H) 7.12-7.29 (m, 5H) 7.29-7.32 (m, 2H) 7.36-7.59 (m 2H) 7.93 (br s, 1H)); LCMS (ESI) m/z 971.9 (M+2H)²⁺.

### Intermediate I

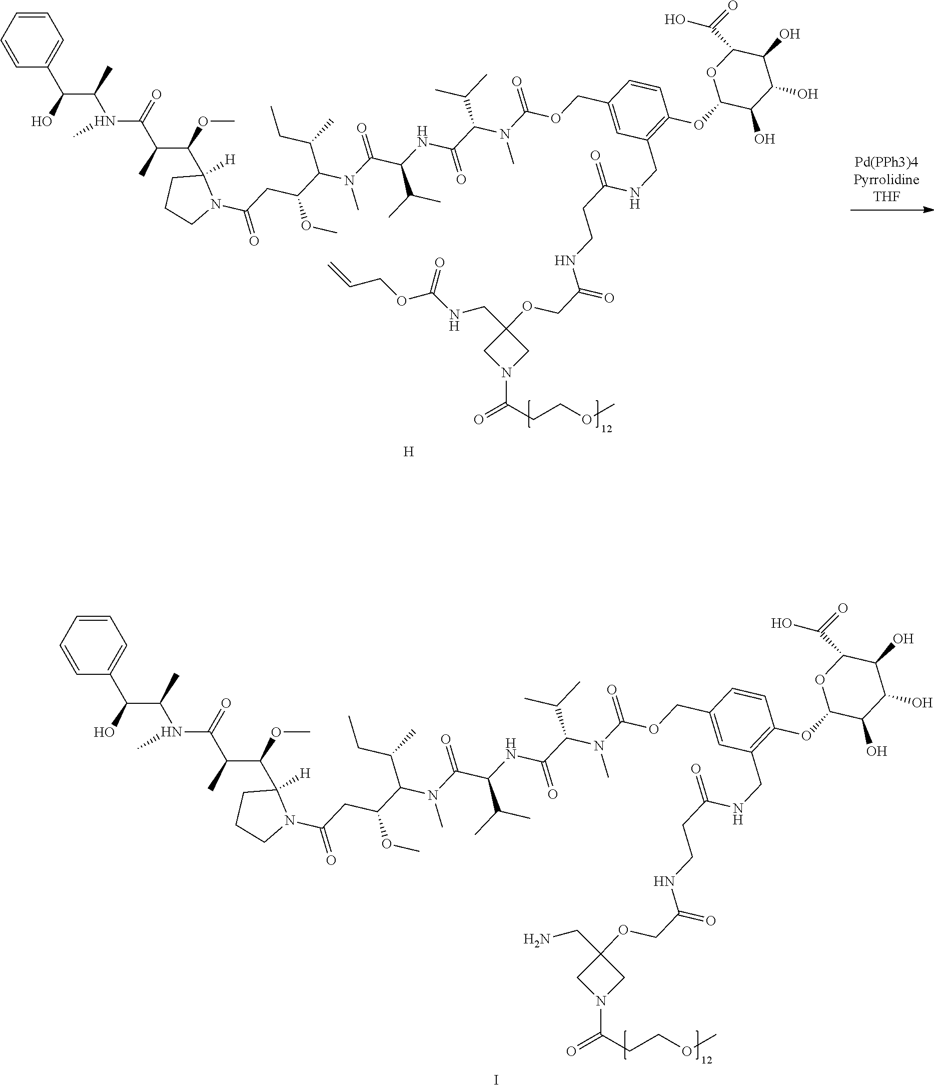

To a stirred solution of Intermediate H (17.1 g, 8.89 mMol, 1.0 eq) in THF (256.5 mL, 15V) at 0° C. was added pyrrolidine (1.57 g, 22.02 mMol, 2.5 eq). The mixture was degassed three times with nitrogen, then Pd(PPh₃)₄ (0.51 g, 0.441 mMol, 0.05 eq) was added. The reaction mixture was stirred at 0° C. for 2 h. Water was charged (171 mL, 10V) and the pH was adjusted to 4-6 by adding HCl (1M aq, 3.4 mL, 0.2 V). The reaction mixture was extracted twice with toluene (2×171 mL, 2×10 V), the layers separated and the aqueous layer retained (note: product in aqueous). The aqueous phase was concentrated to dryness by freeze-drying to give (2~{S},3~{S},4~{S},5~{R},6~{S})-6-[2-[[3-[[2-[3-(aminomethyl)-1-[3-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]propanoyl]azetidin-3-yl]oxyacetyl]amino]propanoylamino]methyl]-4-[[[(1~{S})-1-[[(1~{S})-1-[[(1~{S}),2~{R})-4-[(2~{S})-2-[(1~{R},2~{R})-3-[[(1~{R},2~{S}))-2-hydroxy-1-methyl-2-phenyl-ethyl]amino]-1-methoxy-2-methyl-3-oxo-propyl]pyrrolidin-1-yl]-2-methoxy-1-[(1~{S})-1-methylpropyl]-4-oxo-butyl]-methyl-carbamoyl]-2-methyl-propyl]carbamoyl]-2-methyl-propyl]-methyl-carbamoyl]oxymethyl]phenoxy]-3,4,5-trihydroxy-tetrahydropyran-2-carboxylic acid, Intermediate I, as a solid (13.0 g, 73.1 w/w %, 75.4% yield).

### LP2 (Alternative Synthesis)

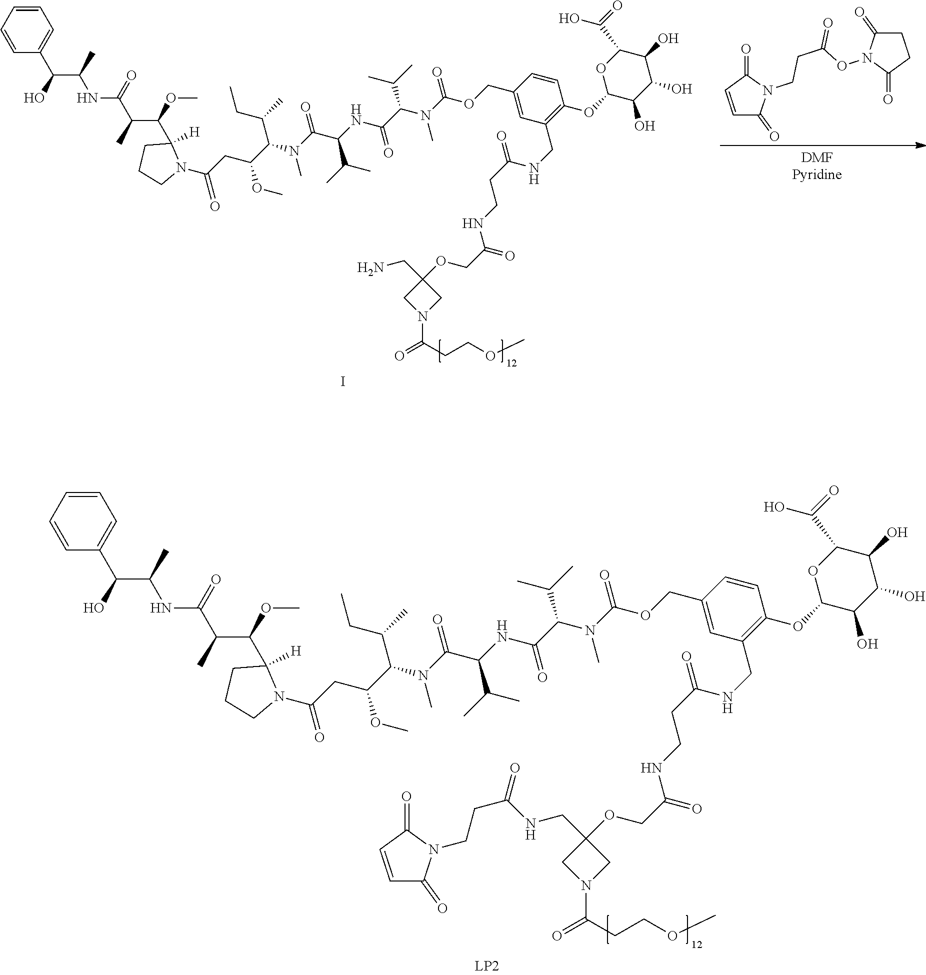

To a stirred solution of Intermediate I (0.75 g, 404 Mol, 1.0 eq) and pyridine (0.38 mL, 0.5 V) in DMF (7.51 mL, 10 V) at 25° C. was added (2,5-dioxopyrrolidin-1-yl) 3-(2,5-dioxopyrrol-1-yl)propanoate (215 mg, 809 Mol, 2.0 eq). The reaction mixture was stirred for 2 h at 25° C. then purified directly by HPLC. Fractions containing the target compound were combined and isolated by freeze drying to give LP2 as a solid (300 mg, 96.7 w/w %, 35.7% yield).

### Example 2: Production and Purification of Anti-B7H3 Antibodies

The anti-B7H3 antibody was discovered using a single-chain Fv human antibody library (Vaughan et al., 1996), derived from B-lymphocytes isolated from tonsil, bone marrow, and peripheral blood, was cloned into a phagemid vector and used for selections. This scFv repertoire is estimated to have approximately 1.3×10¹⁰ unique individual recombinants phage. ScFvs from this library include a C-terminal stretch of 6 histidines tag to enable scFv purification via immobilized metal affinity chromatography (IMAC) and a short sequence from c-myc, allowing detection using the monoclonal anti-c-myc antibody, 9E10. The huB7H3 antigen was biotinylated with Sulfo-NHS-LC-biotin (Pierce) following the manufacturer's recommendations. Dynal M280 magnetic beads were coupled to the biotinylated antigen, and unbound antigen was removed by washing three times with PBST (PBS containing 0.1% Tween 20). The antigen-coupled beads were blocked with 3% powdered milk in 1× phosphate-buffered saline with 0.1% Tween 20 (3% MPBS) at 37° C. for 2 hours. After washing with PBST, 500 μl of phagemid particles (10¹³ tu) in 3% MPBS was added and incubated at 37° C. for 1 hour. The solution was decanted, and the beads were washed 10 times with PBST (1× phosphate-buffered saline with 0.1% Tween 20) and 10 times with 1×PBS. The bound phage was eluted with 1 ml 100 mM triethylamine for 10 minutes at room temperature, then neutralized with 0.5 ml 1M Tris-HCl (pH 7.4). This eluted phage was used to grow exponentially with 10 ml of E. coli TG1. Infected cells were cultured in 2YT broth for 1 hour at 37° C. with light aeration and then plated on 2×TYAG agar plates and incubated overnight at 30° C. Colonies were scraped from the plates and placed in 10 ml of 2YT broth, with 15% glycerol added for storage at −80° C. The glycerol stock culture from the first round of panning on the B7H3 protein was superinfected with helper phage and rescued for a second round of panning. For this, 25 μl of glycerol stock was added to 25 ml of 2TYAG broth, seeded, and incubated at 37° C. with good aeration until OD 600 nm reached 0.7. M13K07 helper phage (moi=10) was added to the culture, incubated for 15 minutes, then shaken for 45 minutes at 37° C. The culture was centrifuged, cells resuspended in 50 ml pre-warmed 2YTAK, and rescue performed overnight at 30° C. Phagemid particles were then concentrated and resuspended in PBS to 10¹³ tu/ml. Two subsequent rounds of panning were performed. Individual E. coli containing phagemids were added at 100 μl in a 96-well plate containing TYAG culture medium. Plates were incubated for 4 hours at 37° C. with shaking. M13K07 helper phage was added, and plates incubated for an additional hour at 37° C., then centrifuged for 10 minutes at 2000 rpm. The supernatant was removed, and the cell pellet was resuspended in 100 μl 2TYAK and incubated overnight at 30° C. with shaking. The following day, plates were centrifuged at 2000 rpm for 10 minutes, and 100 μl of phage-containing supernatant from each well was transferred to a new 96-well plate. Three rounds of panning were performed on hB7H3 4Ig ECD with 100 nM, 50 nM, and 25 nM of antigen. Unique scFv were expressed again in bacteria and purified by affinity chromatography (as described in WO 01/66754). ScFv from phage panning rounds 2 and 3 were tested for binding to human and cyno B7H3-ECD by homogeneous time-resolved fluorescence (HTRF) and checked for specificity against an irrelevant antigen. 78% of selected clones bound both human 4Ig & 2Ig isoforms, and 83% showed cyno 4Ig cross-reactivity. Sequencing of the phagemid DNA identified high sequence diversity. Binding was confirmed against B7H3-expressing cell lines, including A375, PC3, A549, DU145, HCT116, HPAFII, MCF7, B7H3-CHO-K1 engineered over-expressing cell lines, and there was no binding to CHO parental cells. The antibody, B7 h3009, binds specifically to human, rodent, murine, and cynomolgus B7H3-EDC. It was affinity optimized through CDR block mutagenesis, with framework residues reverted to the closest human germline sequences (based on the IMGT repertoire) to minimize immunogenicity in humans. All framework residues of the VL and VH domains were reverted to match human sequences. The VL and VH were converted to an IgG1 triple mutant antibody format (“IgG-TM”, IgG1 Fc sequence incorporating mutations L234F, L235E, and P331S). The effector function-null IgG1 TM antibodies were expressed in mammalian cells, purified by affinity chromatography, and ranked based on binding and functional assays. An OriP fragment was included in expression vectors to facilitate use with CHO-transient cells and allow episomal replication. The VH domain was cloned into a vector (pEU1.4) with human heavy chain constant domains and regulatory elements to express the IgG1 TM heavy chain in mammalian cells. Similarly, the VL domain was cloned into a vector (pEU4.4) for the expression of the human light chain (lambda) constant domains and regulatory elements in mammalian cells. To produce IgGs, heavy and light chain vectors were transfected into CHO-transient mammalian cells. IgGs were expressed and secreted into the medium, harvested, and filtered prior to purification. IgGs were purified using Protein A chromatography, loaded onto a column with Ceramic Protein A, washed, and eluted with 0.1 M Sodium Citrate (pH 3.0), then neutralized with Tris-HCl (pH 9.0). Eluted materials were buffer exchanged into PBS and the IgG concentration determined spectrophotometrically. Purified IgG was analyzed for aggregation and degradation purity using SEC-HPLC. The anti-B7H3 IgG-TM antibody (comprising a heavy chain sequence according to SEQ ID NO: 10 and a light chain sequence according to SEQ ID NO: 11; herein referred to as mAb-1) was used to generate the stochastic DAR4 ADC (ADC-1) as described in Example 5.

To generate the site-specific DAR2 ADC (ADC-2; see Example 5), a leucine amino acid at position L174 in the heavy chain CH1 region, using the EU numbering system (L178 in Kabat numbering system), of the anti-B7H3 IgG-TM antibody was mutated to a cysteine residue (providing an anti-B7H3 IgG-TM antibody comprising a heavy chain sequence according to SEQ ID NO: 9 and a light chain sequence according to SEQ ID NO: 11; herein referred to as mAb-2) to facilitate site-specific conjugation with a Drug-to-Antibody Ratio (DAR) of two, using a maleimide-containing linker-payload. The L174 position is proximal to the cavity formed in the Fab region by the pairing of the antibody's heavy and light chains. Conjugation of LP2 at this position results in a stable Antibody-Drug-Conjugate (ADC).

### Example 3: Binding Affinity of Anti-B7H3 Antibodies

The antibody binding affinities of mAb-1 to the B7H3 4Ig and 2Ig isoforms from various species were determined using surface plasmon resonance (SPR) technology in a BIACORE® 2000 instrument using the predetermined antigen, e.g. human B7H3, as the analyte and the antibody as the ligand to determine K_{off}/Kₒₙ rate and resulting Kd of human and cyno B7-H3 (4Ig and 2Ig isoforms) and the mouse and rat 2Ig. Anti-hu IgG-Fc was immobilized to a net response of 8000 RU onto a Series S CM5 sensor chip prior to capture of the anti-B7-H3 antibody and titration of the various B7H3 antigens were made over a concentration region. The ligands used in the experiment, huB7H3-His, cyByH3-His and muB7H3-His were purchased from AcroBiosystem; the rat B7H3-His was acquired from Sino Biologicals. Table 3 shows the determine K_{off}/Kₒₙ rate and resulting Kd values.

TABLE 3
Binding Affinity Data

| Sample | kₐ (1/Ms) | k_{d} (1/s) | K_{D} (K) | Rmax (RU) | Chi² (RU²) |
|---|---|---|---|---|---|
| Hu B7H3 | 2.61E+05 | 1.04E−02 | 40 | 46 | 0.113 |
| Cy B7H3 | 2.35E+05 | 2.27E+04 | 0.97 | 28 | 0.0311 |
| Mu B7H3 | 5.31E+05 | 1.32E−01 | 248 | 43 | 0.345 |
| Mu B7H3 | Steady State | | 261 | 44 | 0.007 |
| Rat B7H3 | 4.52E+05 | 1.78E−01 | 393 | 46 | 0.076 |

| | | | | |
|---|---|---|---|---|
| Rat B7H3 | Steady State | 399 | 49 | 0.108 |

### Example 4: Physicochemical Properties of Anti-B7H3 Antibodies

Generating a therapeutic antibody that is developable and aligned to manufacturing requirements requires a thorough assessment of an antibody's physicochemical properties. mAb-1 was tested in the experiments described below.

### Baculovirus Particle Binding

96 well Nunc Maxisorp F plates; baculovirus particles; 50 mM sodium carbonate coating buffer; PBS; PBS with 0.5% BSA (Gibco 14190086 with Sigma A9576); test antibodies; AGRP0022 positive control (provided as 1 mg/mL aliquots); NIP228 negative control; antihuman IgG HRP (Fcspecific, Sigma A0170); TMB substrate (SureBlue Reserve, KPL 530003); 0.5 M sulfuric acid (H2SO₄) stop solution; plate washer or manual washing setup; EnVision plate reader or equivalent configured for 450 nm absorbance; calibrated pipettes and sterile tips were prepared. Experimental plates were coated by diluting baculovirus particles to 1.0×10^{\hat{ }10} particles/mL in 50 mM sodium carbonate buffer and dispensing 50 μL per well to achieve 5.0×10^{\hat{ }8} particles per well. For no antigen control plates, buffer was dispensed at 50 μL per well. The plates were covered and incubated overnight at 4° C. to allow adsorption of the particles or buffer onto the plate surface. On the following day, the coated and control plates were washed once with PBS to remove unbound material. The wells were blocked by adding PBS with 0.5% BSA at 300 μL per well and incubated for 1 hour at room temperature to minimize nonspecific interactions. After blocking, the plates were washed three times with PBS to remove residual blocker before antibody incubation. All ELISA washes throughout the procedure were performed with PBS only. The tested antibodies were prepared at 10 nM or 100 nM in PBS with 0.5% BSA, and set up each concentration in duplicate wells on both the baculovirus coated plate and the noantigen control plate. AGRP0022 was used as the positive control and NIP228 was used as the negative control, prepared in the same diluent. 50 μL of each antibody solution was added per well and incubated for 1 hour at room temperature to allow binding. Upon completion, the plates were washed three times with PBS to remove unbound antibodies. antihuman IgG HRP (Fcspecific, Sigma A0170) was prepared at a 1:5000 dilution in PBS with 0.5% BSA. 50 μL was added per well and incubated for 1 hour at room temperature to detect bound human IgG. The plates were washed three times with PBS to remove unbound conjugate. 50 μL per well of TMB substrate (SureBlue Reserve) was added to develop color, & the reaction was monitored to ensure signals remained within the dynamic range. The reaction was stopped by adding 50 μL per well of 0.5 M H₂SO₄. The plates were read on an EnVision plate reader or equivalent using the 450 nm absorbance setting for 96 well plates. The absorbance values for each antibody concentration were recorded and replicated on both the baculovirus coated and control plates, ensuring that controls met expected ranges before proceeding to analysis. For each antibody sample, the 450 nm absorbance values at 10 nM and 100 nM were averaged on the baculovirus coated plate, and the corresponding values on the blank control plate were separately averaged. Averages were normalised by dividing by the AGRP0022 baculovirus plate absorbance (i.e., the baculovirus average for the sample was divided by the AGRP0022 baculovirus average, the same calculation was followed for the blank average). The BV score was calculated by dividing the normalized baculovirus result by the normalized blank result for the same antibody. A BV score above 5 may indicate a risk of increased clearance due to nonspecific binding; AGRP0022 should yield a BV score of 30 or greater, reflecting strong nonspecific binding and a very short half life without other developability flags, whereas NIP228 should yield a BV score below 5.

### HEK Cell Binding

The test antibodies were prepared in a 384 well source plate by diluting each to four times the intended final in well concentration in 1×HBSS with 0.5% BSA, dispensing 10 μL per assay well. A fivefold serial dilution series was prepared to achieve final in well concentrations descending from 100 nM, and the number of dilution points were adjusted to suit the assay design. All dilutions used the same buffer to maintain consistent matrix conditions. The detection antibody was prepared by diluting Alexa Fluor 647 Goat antihuman or antimouse IgG (H+L) to four times the required final concentration in 1×HBSS with 0.5% BSA, dispensing 10 μL per assay well. 4 nM was used as the standard final concentration per well. The fluorescent antibody was protected from light during preparation and use. The assay plate was set up in the specified order without mixing between additions. 10 μL of the detection antibody solution was added to each well. 10 μL of the diluted test antibody solution was added to each well. 20 μL of prepared HEK293 cells was added to each well to reach the required cell number per well, or buffer was added instead of cells to designated no cells control wells. The plate was sealed with a TopSeal (PerkinElmer 6050185) to prevent evaporation and contamination. The sealed plate were incubated for two hours at room temperature or according to the assay standard operating procedure to allow binding to reach equilibrium. Using the Mirrorball fluorescence cytometer, the detector was configured for the Alexa Fluor 647 channel (FL3). Data acquisition parameters were set with a minimum count (MIN COUNT) threshold of 25 (lower cutoff) and the FL3 PMT gain at 500 V. Wells containing only the detection antibody served as the baseline to determine background fluorescence. Non-specific binding was quantified by normalizing the fluorescence intensity of the test samples as a percentage relative to the control signal.

### Accelerated Stability (2 Weeks at 45′)

Each IgG sample was adjusted to a concentration between 0.5 and 1.0 mg/mL in DPBS. The samples were vortexed gently or inverted to mix, and clarity assessed before stressing. An unstressed control aliquot was prepared at the same concentration to serve as a visual reference. 200 μL of each prepared sample was dispensed into sterile 0.6 mL Eppendorf tubes. The tubes were capped securely to minimize evaporation while allowing for potential thermal expansion. The capped tubes were placed in a temperature controlled incubator set to 45° C. & incubated for two weeks without disturbance. After incubation, the samples were allowed to return to room temperature, and each stressed sample was inspected alongside its matched unstressed control. The samples were assessed for the presence of visible particles, increased turbidity, or color changes against a light background, and any precipitation or flocculation were noted. The stressed samples to were filtered to remove particulates using an appropriate low protein binding membrane filter and pore size, collecting the filtrate into clean, labelled recipient tubes. Changes in binding activity between stressed and non-stressed material were measured using a HTRF binding assay.

### Results

The results of the above experiments are summarised in Table 4:

TABLE 4
Results of developability experiments

| Experiment | Developability criteria | mAb-1 result |
|---|---|---|
| Baculovirus | BV score <5 and no significant binding | 1.9 |
| particle | to uncoated surface | |
| binding | | |
| HEK cell | No significant binding to untransfected | No binding |
| binding | HEK293X cells | |
| Accelerated | Should not exhibit greater than 4% loss | 0.6% monomer |
| stability | of monomer to fragmentation and/or 1% | loss due to |
| (2 weeks | to aggregation | fragmentation; |
| at 45° C.) | | 0.7% loss due |
| | | to aggregation |
| | No marked change in activity (>2 fold | No loss in |
| | loss) or clear downward trend in | activity |
| | stressed sample relative to unstressed | (binding) |

The results indicate that mAb-1 is suitable for further development.

### Example 5: Production and Purification of Anti-B7H3 Antibody-Drug Conjugates

### ADC-1—Stochastic DAR4 ADC

As described in Example 2, mAb-1 (comprising a heavy chain sequence according to SEQ ID NO: 10 and a light chain sequence according to SEQ ID NO: 11) was used to generate the stochastic DAR4 ADC (ADC-1).

A 5 mM solution of Tris(2-carboxyethyl)phosphine (TCEP, Pierce) in phosphate buffered saline pH 7.4 (PBS, Gibco) was added (2.7 molar equivalent/antibody, 542 nanomoles, 11 μl) to a 6 ml solution of antibody (mAb-1, 30 mg each, 200 nanomoles) in reduction buffer containing PBS and 1 mM ethylenediaminetetraacetic acid (EDTA, Molekula) and 10% v/v of TRIS pH 8.5 (667 uL) to a final antibody concentration of 5 mg/ml. The reduction mixture was heated at +37° C. for 3 hours in an incubated orbital shaker with gentle (60 rpm) shaking. After cooling down to room temperature, LP2 was added as a DMSO solution (7 molar equivalent/antibody, 1.40 micromoles, in 0.57 ml DMSO) to this reduced antibody solution for a 10% (v/v) final DMSO concentration. The solution was mixed for 2 hours at room temperature, then the conjugation was quenched by addition of N-acetyl cysteine (7.1 micromoles, 71 μl at 100 mM) for >30 minutes at room temperature, sterile filtered, then purified on an AKTA™ Start FPLC using a GE Healthcare HiLoad™ 26/600 column packed with Superdex 200 PG, eluting with 2.6 ml/min PBS. Fractions corresponding to ADC monomer peak were pooled, concentrated using a 15 ml Amicon Ultracell 30 KDa MWCO spin filter, sterile-filtered and analysed.

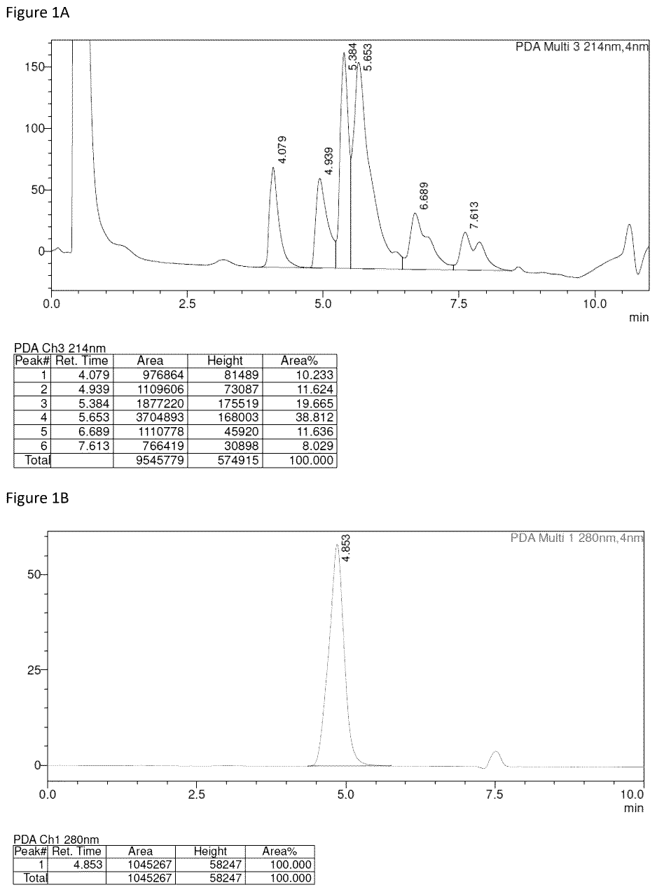

UHPLC-RP analysis on a Shimadzu Prominence system using a 5 Thermo Scientific MAbPac 50 mm×2.1 mm column eluting with a gradient of water and acetonitrile on a reduced sample of ADC at 214 nm and 254 nm showed a mixture of unconjugated light chains (L0), light chains attached to a single molecule of LP2 (L1), unconjugated heavy chains (H0) and heavy chains attached to up to three molecules of LP2 (H1, H2, H3), consistent with a drug-per-antibody ratio (DAR) of 3.77 molecules of LP2 per antibody, calculated using similar methods as described for ADC-2. Free NAC-quenched payload was <LOD. A representative UHPLC-RP chromatogram for ADC-1 is shown in FIG. 1A, which demonstrated a DAR of 3.77.

UHPLC-SEC analysis on a Shimadzu Prominence system using a Tosoh Bioscience TSKgel SuperSW mAb HTP 4 μm 4.6×150 mm column (with a 4 μm 3.0×20 mm guard column) eluting with 0.3 ml/minute sterile-filtered SEC buffer containing 200 mM potassium phosphate pH 6.95, 250 mM potassium chloride and 10% isopropanol (v/v) on a sample of ADC at 280 nm showed a monomer purity of >99.5%. A representative UHPLC-SEC chromatogram for ADC-1 is shown in FIG. 1B.

UV (Stunner, Unchained Labs) analysis gave a concentration of final ADC at 1.66 mg/ml in 11 ml, obtained mass of ADC of 18.3 mg (61% yield).

### ADC-2—Site-Specific DAR2 ADC

As described in Example 2, mAb-2 (comprising a heavy chain sequence according to SEQ ID NO: 9 and a light chain sequence according to SEQ ID NO: 11) was used to generate the site-specific DAR2 ADC (ADC-2).

4 g (318 mL at 12.6 mg/mL) of antibody (mAb-2) in 1×PBS, pH 7.2 supplemented with 1 mM ethylenediaminetetraacetic acid (EDTA, Invitrogen) was reduced using 50 molar equivalents (2.33 ml) of 0.5M Tris-(2-carboxyethyl)-phosphine (TCEP), Pierce. Additionally, conjugation buffer (1×PBS, pH 7.2, 1 mM EDTA) was added to bring the total volume to 500 mL. The mixture was incubated for 2 h at 37° C. with gentle rotation using a stir bar. The antibody was then dialyzed using a 10 kd MWCO dialysis cassette (Invitrogen) overnight in 10 liters of conjugation buffer to remove the TCEP. The antibody was then incubated with 60 molar equivalents of dehydroasorbic acid for four hours at 25° C. before dialysis overnight as before. 27 ml of DMSO and 8.5 molar equivalents (22.7 ml of 10 mM of the linker-payload in DMSO) of LP2 were added creating a 10% DMSO final concentration. The reaction was allowed to proceed at 25° C. for 3 hours before being checked for completeness by LCMS. The reaction was quenched by the addition of 4 molar equivalents or 90 mL of 100 mM (over linker-payload) of N-acetyl cysteine. The unconjugated linker-payload was removed via tangential flow filtration unit (mPES, MidiKros 30-kDa fiber filter with a 375-cm² surface area) using 10 diavolumes of 20 mM Histidine buffer with 150 mM NaCl, pH 6.0, followed by 10 diavolumes of 20 mM Histidine buffer, pH 6.0). A concentrated stock of sucrose was spiked into the mixture to create the final formulation buffer of 20 mM histidine, 240 mM sucrose, 0.02% PS-80, pH 6.0. The resulting ADC was analyzed by SEC-HPLC for monomer, HPLC-HIC for hydrophobicity, LCMS for drug loading and rP-HPLC for the free linker-payload. The final concentration of the ADC, by UV absorbance (Nanodrop, Thermo), was 10.6 mg/mL with a final yield of ~86%.

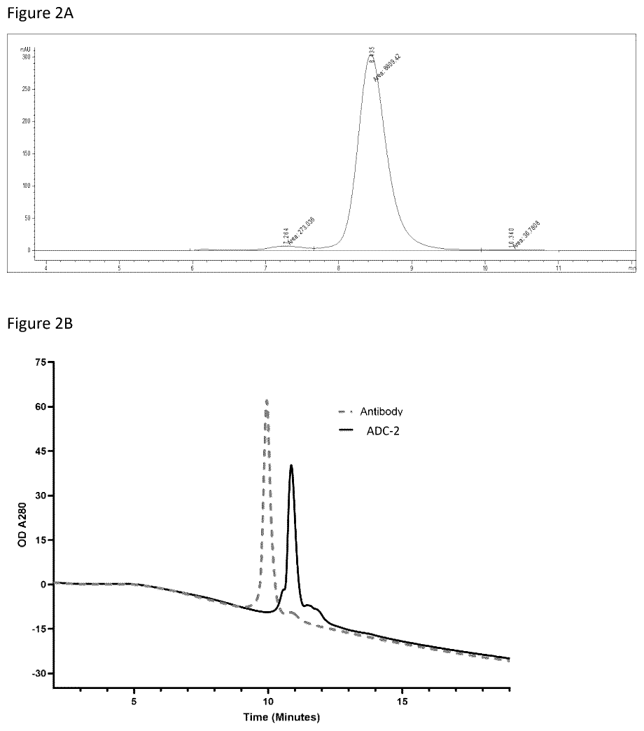

To determine the monomer content of the starting antibody and ADC, 100 g of sample was injected onto an Agilent 1290 Capillary UHPLC System equipped with a UV detector and a HPLC-SEC TSK-GEL G3000SWXL column (Tosoh Bioscience LLC, Montgomeryville, PA). The run buffer consisted of 200 mM sodium phosphate (pH 6.8) with 10% isopropanol. The sample ran over the column at a flow rate of 1 mL/min for a total run time of 20 minutes. The monomer content of the starting antibody was determined to be 98%, and that of ADC (ADC-2) was also determined to be 96.5% monomer with 3.06% aggregate and 0.41% fragment, as shown in FIG. 2A.

Hydrophobic interaction chromatography (HIC-HPLC) was employed to determine conjugation efficiency and drug load distribution. A Butyl-non-porous resin (NPR) column (4.6 μm ID×3.5 cm, 2.5 μm, Tosoh Bioscience) was used with running buffers of 25 mM Tris(hydroxymethyl)aminomethane hydrochloride (Invitrogen), 1.5 M Ammonium sulfate (Sigma), pH 7.0 (mobile phase A) and 25 mM Tris(hydroxymethyl)aminomethane hydrochloride, 5% isopropanol, pH 7.0 (mobile phase B). A solution of 10 μL of antibody or ADC at a concentration of 1 mg/mL was loaded in duplicate and eluted at a flow rate of 0.8 mL/min with a gradient of 5% to 100% mobile phase B over 13 min, while detecting the absorbance at 280 nm. As shown in FIG. 2B, the antibody and ADC have HIC retention times of 9.98 and 10.793 minutes, respectively.

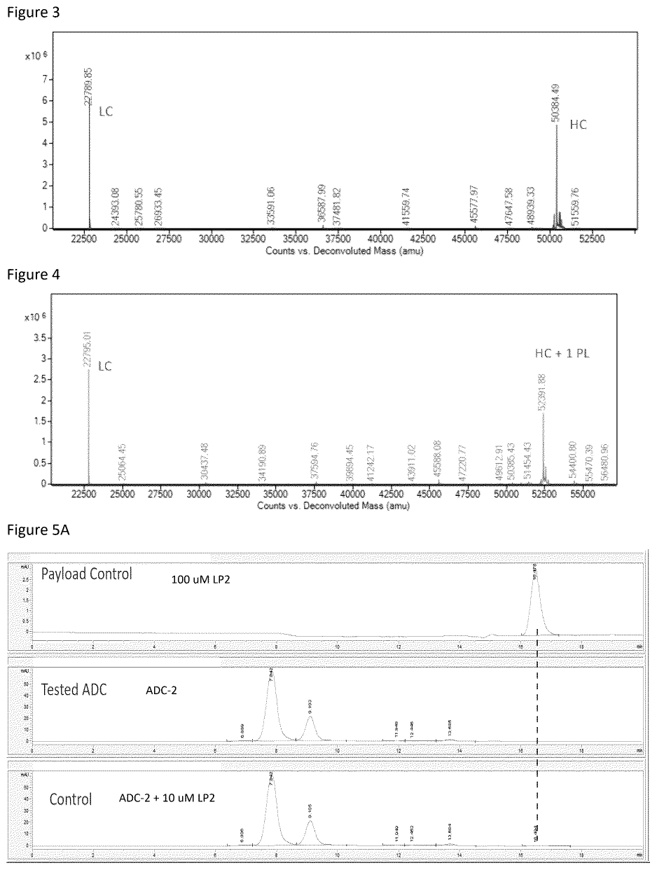

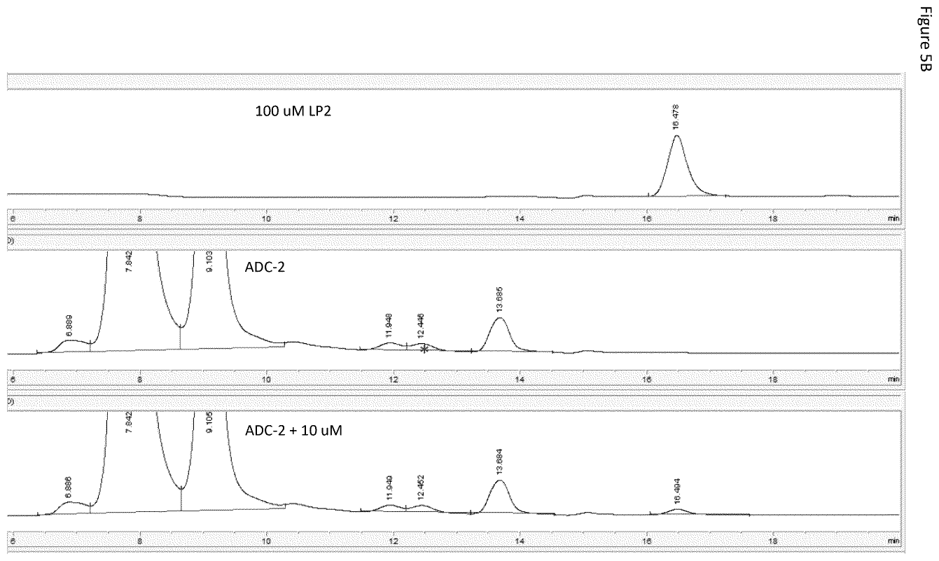

The efficiency of conjugation and Drug-to-Antibody-Ratio (DAR) were determined using reduced reverse phase liquid chromatography mass spectrometry (rLCMS). rLCMS was carried out on an Agilent 1200 series HPLC coupled to an Agilent 6520 Accurate-Mass Q-TOF LC/MS with an electrospray ionization source. A solution of 45 μL of 1 mg/mL of mAb or ADC was deglycosylated with 2 μL of endoglycosidase S (New England BioLabs) for 1 hour at 37° C. The mixture was then reduced by the addition of 5 μL of 100 mM Dithiothreitol (Invitrogen) and incubated at 37° C. for 20 minutes. 2 μL of reduced antibody or ADC was injected into the system while running a 10% mobile phase A at 0.4 mL/min into a Poroshell 300SB-C3 column (2.1×75 mm, Agilent). The sample was eluted using a step gradient of 60% B after 2 min (mobile phase A: 0.1% Formic acid in water and mobile phase B: 0.1% Formic acid in acetonitrile (JT Baker)) directly into the mass spectrometry. Agilent Mass Hunter was used for data acquisition and chromatogram processing. The Drug-to-antibody-ratio of ADC-2 was calculated using the following equation:

$$DAR = 2 \times ( ( \frac{( LC + 1 )}{( ( LC + 0 ) + ( LC + 1 )} ) + ( \frac{( HC + 1 ) \times 1 + ( HC + 2 ) \times 2 + ( HC + 3 ) \times 3}{( HC + 0 ) + ( HC + 1 ) + ( HC + 2 ) + ( HC + 3 )} ) )$$

where (LC+n) is the peak height of the light chain with 0 or 1 drug, and (HC+n) is the peak height of the heavy chain with 0 or 1 drugs. The antibody and ADC-2 chromatographies are shown in FIGS. 3 and 4, respectively. ADC-2 was determined to have a DAR of 2 linker-payloads per mAb.

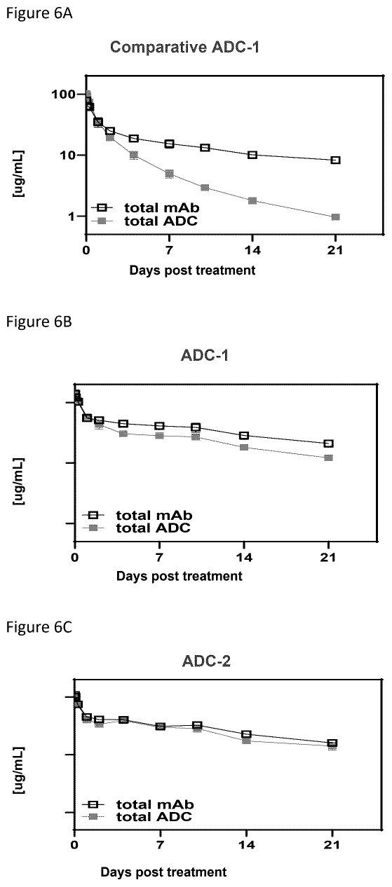

An Agilent 1290 UHPLC system equipped with an auto-sampler and DAD was used to determine the percentage of N-acetyl cysteine-quenched linker-payload in the formulated ADC samples. To generate the standard curve for the determination of free drug, a series of N-acetyl cysteine-quenched linker-payload dilutions of a 100 M solution in 100% DMSO was used. 2 μL each ADC at 10.6 mg/mL or varying amounts of standard from 0.1-5 uL was injected onto a Waters BioResolve Polyphenyl 2.1×150 mm column and eluted at 50° C. at a flow rate of 0.4 mL/min with a gradient of 5% to 25% B over 0.5 min followed by 25% to 50 from 7.5 minutes (mobile phase A: 0.1% Trifluoroacetic acid in water; mobile phase B: 0.1% Trifluoroacetic acid in acetonitrile). The standard curve generated based on the absorbance of the free drug at 280 nm was used to calculate the amount of residual payload in each ADC sample. The amount of free drug is reported as mole percent (%) of total free drug related to the amount of conjugated drug. The chromatographs are shown in FIGS. 5A and 5B.

### Comparative ADC-1

Comparative ADC-1 was prepared using analogous methods to those described for ADC-1, using LP1 instead of LP2. UHPLC-RP analysis as described provided results which were consistent with an average drug-per-antibody (DAR) of 4 LP1 per antibody (data not shown).

### Example 6: Pharmacokinetics Profile of Anti-B7H3 Antibody-Drug Conjugates

Male NSG mice (n=3 per group) were dosed intravenously (bolus) with ADC-1, ADC-2 and comparative ADC-1 (5 mg/kg). Blood samples were taken at 30 min, 2 h, 6 h, 1, 2, 4, 7, 10, 14 and 21 days after dosing, centrifuged to obtain plasma and analyzed for total ADC. Pharmacokinetic parameters were determined using Phoenix 64 software (Certara).

Total ADC concentrations were measured with an immuno-capture LC-MS/MS assay. Briefly, a polyclonal anti-human antibody was conjugated to magnetic beads. Then 10 μL of plasma sample was diluted in TBS and incubated together with the magnetic beads. After capturing, the magnetic beads were washed multiple times before digestion with trypsin. An aliquot of the trypsin digested supernatant was subjected to enzymatic digestion with papain followed by a further overnight digestion with β-glucuronidase, in the presence of an internal standard. The digested samples were then precipitated using acidified acetonitrile, with the resulting supernatant evaporated under nitrogen. The extracts were reconstituted in a suitable solvent before analysis by LC-MS/MS.

The β-glucuronidase released payload was used to calculate the concentration of ADC. The internal standard used in this experiment is the isotopically labeled payload. The β-glucuronidase digest from the immuno-affinity enriched samples was separated using reversed phase liquid chromatography (RPLC) followed by detection using multiple reaction monitoring (MRM) for the released payload. The peak area ratio of the analyte against the internal standard was used to calculate against the standard curve, which was created by spiking the ADC reference material in the desired matrix.

The standard curves and QCs were prepared by spiking the target compound (the ADC reference material) at different levels into the same matrix as the sample matrix. The quantification range covers 0.500 μg/mL—60.0 μg/mL, with the dilution QC covering up to 20-fold dilution. The standard curve was fitted with a linear regression, with weighting of 1/×2.

### Results

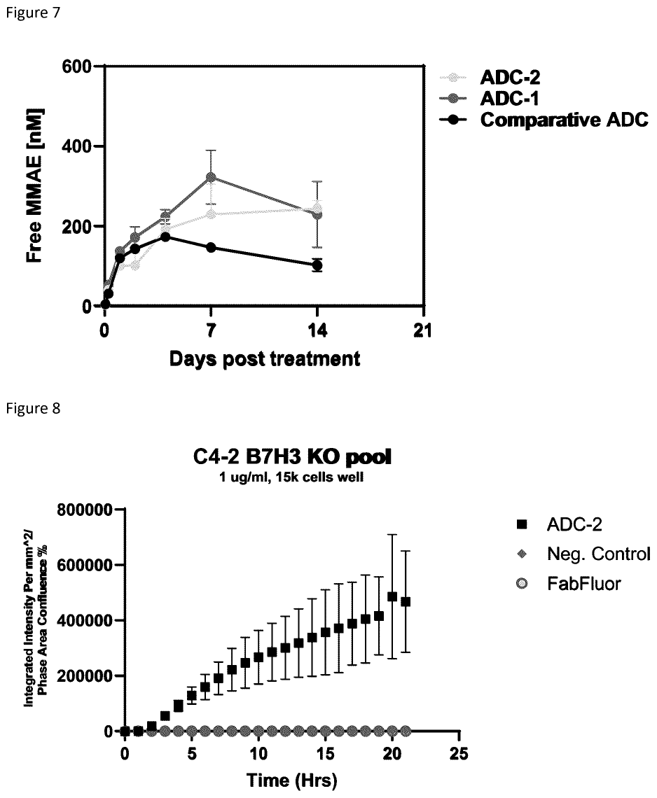

Total mAb and total ADC are shown in FIGS. 6A-C for each ADC. The difference between total mAb and total ADC at each time point reflects the deconjugation of the payload. The pharmacokinetic parameters for ADC-1, ADC-2 and Comparative ADC-1 in NSG mice are shown in Table 5.

TABLE 5
Pharmacokinetic parameters for ADC-1,
ADC-2 and comparative ADC-1 in NSG mice

| | Cₘₐₓ | AUCₗₐₛₜ | CL | Vₛₛ | t_{1/2} |
|---|---|---|---|---|---|
| | [μg/ml] | [day*μg/ml] | [ml/kg/day] | [ml/kg] | [day] |
| ADC-1 | 146.3 | 580 | 6.3 | 100 | 12.1 |
| ADC-2 | 98.7 | 564 | 6.0 | 105 | 11.3 |
| Comparative | 102.7 | 168.5 | 28 | 152 | 7.8 |
| ADC-1 | | | | | |

Cₘₐₓ = maximum observed plasma concentration; AUCₗₐₛₜ = area under the plasma concentration versus time curve to the last measurable timepoint; CL = plasma clearance; Vₛₛ = volume of distribution at steady state; t_{1/2} = elimination phase half-life.

### Example 7: Pharmacodynamic Study of ADC-1, ADC-2 and Comparative ADC-1 in the DU145 Xenograft Model

Immunodeficient male NSG mice were inoculated subcutaneously (SC) with 5×10⁶ cells of the human prostate cancer cell line DU145 and after tumors grew in volume to approximately 250 to 300 mm3, animals were randomized, and each mouse received an IV injection of ADC. Accounting for DAR differences, animals received either 2.5 mg/kg (ADC-1, comparative ADC-1) or 5 mg/kg (ADC-2). Tumors were collected at 1 h, 6 h, 1, 2, 4, 7, 10 and 14 days after dosing.

Deconjugated payload concentrations in tumor tissue were measured using LC-MS/MS analysis. Briefly, the collected tumour tissues were homogenized in RIPA buffer fortified with protease inhibitors (1:5 w/v). 25 μL of the tumour samples were then extracted alongside plasma calibration standards and QC samples, by protein precipitation in the presence of a labelled internal standard MMAE-D8. The supernatants from the extraction were evaporated under nitrogen before being reconstituted in a suitable solvent prior to LC-MS/MS analysis. The LLOQ was 0.1 nM and ULOQ was 100 nM. Pharmacokinetic parameters were determined using Phoenix 64 software (Certara).

### Results

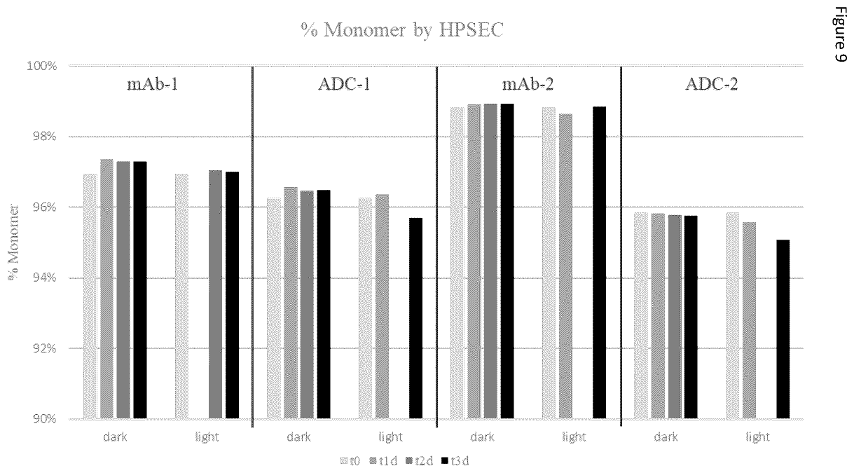

As shown in FIG. 7, accumulation of deconjugated MMAE was observed in tumor tissue over time. Table 6 shows the concentrations of deconjugated MMAE as measured by AUCₗₐₛₜ in DU145 tumors.

TABLE 6
Tumor MMAE concentration for ADC-1, ADC-2
and Comparative ADC-1 in DU145 tumors

| | Dose [mg/kg] | Tumor MMAE AUCₗₐₛₜ [nM · h] |
|---|---|---|
| ADC-1 | 2.5 | 80 |
| ADC-2 | 5 | 66 |
| Comparative ADC-1 | 2.5 | 44 |

### Example 8: Internalisation Evaluation of ADC-2

The IncuCyte fluorescent labeling reagent comprises a Fab fragment that specifically binds the Fc region of the test antibody and is conjugated to a pH-sensitive fluorophore that increases fluorescence upon acidification. Upon binding of the fluorescently labeled Fab to the test antibody, and subsequent engagement of the antibody with the B7-H3 receptor on a target cell, receptor-mediated internalization occurs followed by intracellular trafficking to acidic endosomal and lysosomal compartments. Acidification within these compartments activates the fluorophore, resulting in an increase in fluorescence. The measured fluorescence intensity is directly proportional to the extent of antibody internalization.

On Day 1, plates were prepared by adding 150 μL of sterile PBS to all outer wells (A1-A12, H1-H12, and the perimeter wells) of the 96 well black bottom plate. The cells were harvested and counted by dissociating them with TrypLE and neutralizing with 10× volume of medium. The cells were counted using a vicel counter. The total number of cells and the volume needed to achieve 15,000 cell/mL was calculated. The cell suspension was prepared in complete medium. The calculated volume of medium was added to a conical tube to achieve the desired final concentration and gently mixed to maintain a uniform suspension while avoiding bubbles. The cells were plated by transferring the calculated volume of cell suspension to the plate according to the plate map, keeping one cell line per plate. 80 μL of the cell suspension was dispensed into each intended well, changing tips as needed to avoid cross contamination. The cells were allowed to settle at the bottom & then transferred to a humidified incubator set to 37° C. with 5% CO₂ and incubate overnight for 16-24 hours prior to the internalization procedure.

On Day 2, the 5× labeling solution was prepared by mixing the tested antibody with the IncuCyte fluorescent labeling Fab at a 1 to 3 mAb/labeled Fab ratio. The plated cell plate from the incubator was retrieved and 20 μL of the 5× labeling solution added to each designated well. The plate was placed into the IncuCyte instrument and allowed to acclimate for approximately 15 minutes for the plate to adjust to the instrument environment. The imaging was configured to acquire images every hour for 16-20 hours using the appropriate phase and fluorescence channels, objective, focus, and exposure settings per the assay requirements, then scanning began and the first images were verified before continuing. The total well fluorescent vs time was plotted using Graphpad.

### Results

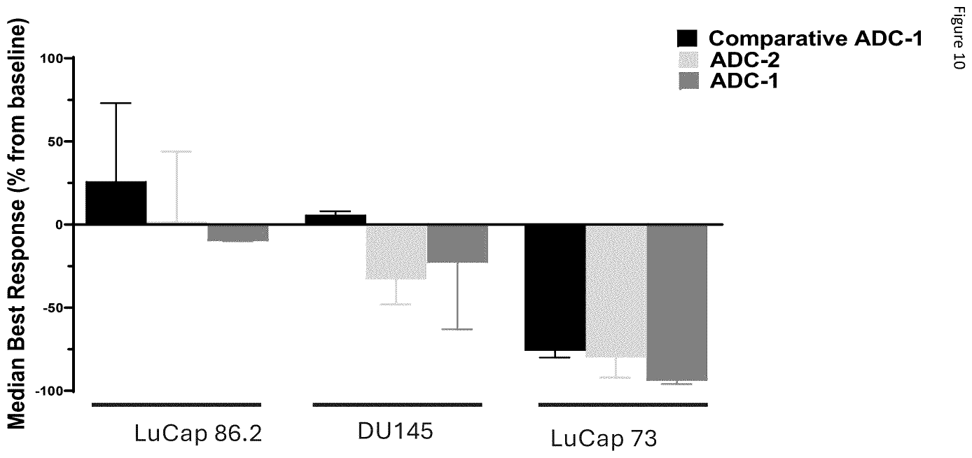

The results of the internalisation assay are shown in FIG. 8.

### Example 9: Developability Assessment of Anti-B7H3 Antibodies and Antibody Drug-Conjugates

### Colloidal Stability Assay

Colloidal stability describes a protein's propensity to form aggregates and precipitate out of solution. The kD values below represent the rate constant for this precipitation process, the lower (more negative) the value, the higher the conjugate's propensity for precipitation. mAb-1, mAb-2, ADC-1 and ADC-2 were concentrated to ~10 mg/mL in 20 mM His/His HCL, 240 mM sucrose pH 6.0. Serial dilutions in the formulation buffer were performed.

### Conformational Stability Assay

Thermal melting transitions were determined by differential scanning calorimetry (DSC) using a MicroCal PEAQ-DSC (Malvern Panalytical). Antibody solutions were diluted to 1 mg/mL in 20 mM histidine/histidine-HCl, 240 mM sucrose, and 0.03% (w/v) polysorbate 80 at pH 6 and change in the heat capacity (Cp) was measured as the samples were heated from 20° to 95° C. using a temperature ramp of 60° C./h. Normalized heat capacity data was obtained after subtracting the buffer blank and normalizing to the antibody concentration. Data were analyzed using MicroCal PEAQ-DSC software (version 1.62) where thermal melting transitions associated with the unfolding of the distinct domains were calculated using a non-two-state model.

### Liquid Stability Assay

mAb-1 and mAb-2 were formulated at 50 mg/mL in 20 mM histidine/histidine-HCl with 240 mM sucrose at pH 6 (no surfactant). ADC-1 and ADC-2 were formulated at 20 mg/mL in 20 mM histidine/histidine-HCl, 240 mM sucrose, and 0.03% (w/v) polysorbate 80 at pH 6. Formulations were filled into HDPE and clear 2R glass vials and were subjected to three freeze-thaw cycles to −80° C. prior to study initiation. Samples were stored at 40° C., 25° C., and 5° C. for 4 weeks with scheduled sampling. Analytical testing included size-exclusion chromatography (HPSEC) for monomer and aggregate, and visual inspection for visible particles, opalescence, and color.

### Photostability Assay

mAb-1 and mAb-2 were formulated at 10 mg/mL in 20 mM histidine/histidine-HCl with 240 mM sucrose at pH 6. ADC-1 and ADC-2 were formulated at 10 mg/mL in 20 mM histidine/histidine-HCl, 240 mM sucrose, and 0.03% (w/v) polysorbate 80 at pH 6. Formulations were filled into clear 2R vials and were exposed to 2,000 lux illumination at 25° C. for 3 days to assess stability. Analytical testing included size-exclusion chromatography (HPSEC) and visual inspection.

### Results—Colloidal Stability

Negative KD values of greater than about −5.34 ml/g indicate attractive interactions, whilst negative KD vales of greater than about −20 ml/g may pose a significant manufacturing risk. Positive KD values indicate repulsive interactions. The results are shown in Table 7.

TABLE 7
Colloidal Stability Results

| | Measured KD at 25° C. (ml/g) |
|---|---|
| mAb-1 | −3.0 |
| ADC-1 | −7.5 |
| mAb-2 | 2.1 |
| ADC-2 | −6.1 |

The results show that mAb-1 and mAb-2 show little to no attractive interactions between molecules, whilst ADC-1 and ADC-2 exhibit slight attractive interactions between molecules. The results support the manufacturability for all the evaluated molecules.

### Results—Conformational Stability

Tm onset values of less than 50° C. are indicative of general conformational instability and temperature sensitivity. All evaluated molecules exhibited a Tm onset of at least 55° C., and none of the melting profiles suggested any thermal stability risk for these molecules. The results are shown in Table 8.

TABLE 8
Conformational Stability Results by DSC

| | Tm onset | Tm1 | Tm2 | Tm3 |
|---|---|---|---|---|
| | (° C.) | (° C.) | (° C.) | (° C.) |
| mAb-1 | 58.24 | 64.76 | 72.66 | 77.77 |
| ADC-1 | 55.70 | 66.89 | 71.75 | 83.55 |
| mAb-2 | 58.26 | 64.08 | 72.54 | 82.23 |
| ADC-2 | 56.76 | 63.89 | 71.10 | 82.99 |

### Results—Liquid Stability

The results of the liquid stability assay are shown in Table 10. All tested molecules exhibited good freeze-thaw stability, and no major changes in visual appearance were observed under any of the tested conditions over a 4-week period. The % monomer loss (rate/month) as measured by HPSEC are shown in Table 9.

TABLE 9
Liquid Stability Results

| Stability Condition | mAb-1 | ADC-1 | mAb-2 | ADC-2 |
|---|---|---|---|---|
| 40° C., clear 2R vial | −3.0 | −9.0 | −0.5 | −3.9 |
| 25° C., clear 2R vial | −0.8 | −1.9 | 0.0 | −1.2 |
| 25° C., HDPE bottle | −0.9 | −1.5 | −0.1 | −0.5 |
| 5° C., clear 2R vial | −0.4 | 0.7 | 0.1 | 0.0 |
| 5° C., HDPE bottle | −0.5 | 0.6 | 0.0 | −0.3 |

The rates of monomer loss for all tested molecules were not considered to be a concern for further development.

### Results—Photostability

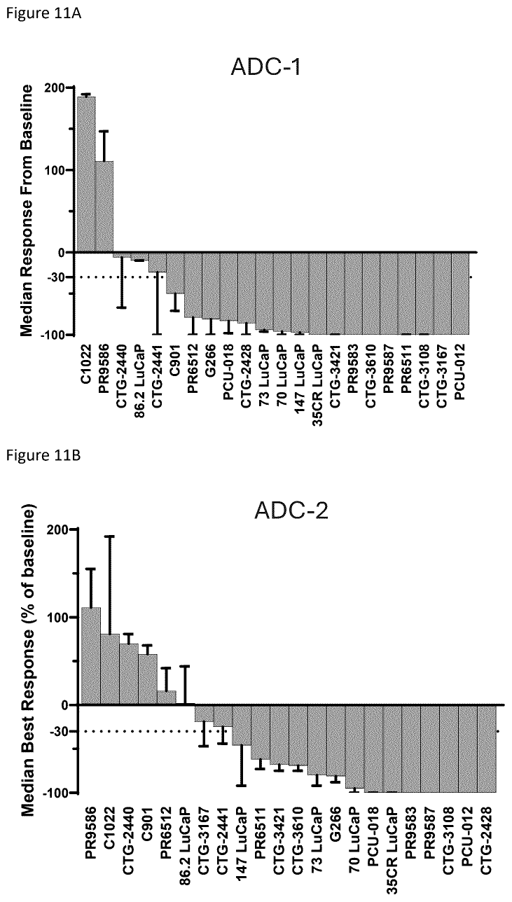

The results of the photostability assay are shown in FIG. 9. All tested molecules exhibited monomer loss rates of less than 0.3% monomer per day, and none exhibited any change in visual appearance. These results indicate that photostability is not a risk for any of mAb-1, mAb-2, ADC-1 or ADC-2, meaning that no additional light exposure mitigation strategies beyond standard protocols are necessary for further development.

### Example 10: Antitumor Efficacy of ADC-1, ADC-2 and Comparative ADC-1 in Subcutaneous Human Prostate Cancer Xenograft Models

For the prostate CDX model DU145, 5×10⁶ cells/mouse were inoculated subcutaneously into male NSG mice. When tumours reached approximately 100-300 mm³, ADCs were administered intravenously with a single dose of 2 mg/kg for ADC-1 and Comparative ADC-1, or 4 mg/kg for ADC-2. For the prostate PDX models LuCap 86.2 and LuCap 73, tumor fragments (size) were subcutaneously transplanted into male NSG mice. When tumours reached approximately 100-300 mm³, mice were allocated to give comparable mean tumor volume between arms. ADCs were administered intravenously with a single dose of 2 mg/kg for ADC-1 and Comparative ADC-1, or 4 mg/kg for ADC-2. Tumor volume was measured twice weekly with calipers during the length of the study. Tumor response was measured as a percentage of tumor volume from baseline. The response at the final tumour volume (FTV) from initial tumour volume (ITV) was calculated at the time that resulted in the greatest decrease from the initial tumour volume. The antitumour response was calculated for each tumour bearing mouse using the formula:

$$Tumour Growth ( \% ) = [ ( F T V - I TV ) / ITV ] \times 100$$

Tumour Growth (%) was calculated for each animal in a study group and the group response was calculated as the median response of all treated mice.

For each treatment group a determination was made whether there was a response or no response to the test agent. Response criteria are based on RECIST 1.1 where a response is based on a 30% decrease in tumour volume from baseline tumour measurements.

### Results

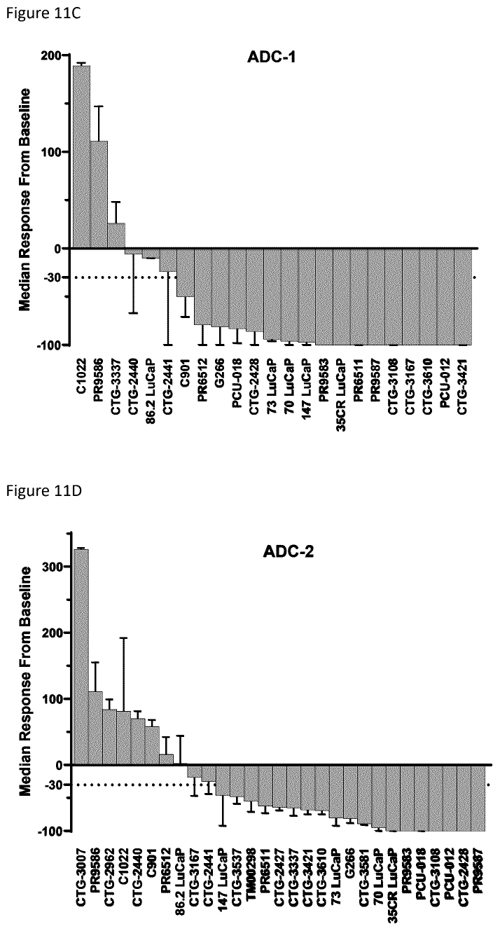

As shown in FIG. 10, variable responses were observed across the 3 prostate models.

### Example 11: Antitumor Efficacy of ADC-1 and ADC-2 in Patient-Derived Xenograft Models of Prostate Cancer

22 prostate PDX models were initially selected for investigation; 29 prostate PDX models were investigated in total. NOG or NPG or NMRI nude or NSG or NXG or NCG mice were implanted subcutaneously with tumour tissue fragments. When tumors reached 100-300 mm³, animals were randomised by tumour volume and received a single IV dose of ADC-1 at 2 mg/kg or ADC-2 at 4 mg/kg in order to account for the DAR difference. Tumor volume was measured twice weekly with calipers during the length of the study. Tumor response was measured as a percentage of tumor volume from baseline. The response at the final tumour volume (FTV) from initial tumour volume (ITV) was calculated at the time that resulted in the greatest decrease from the initial tumour volume. The antitumour response was calculated for each tumour bearing mouse using the formula:

$$Tumour Growth ( \% ) = [ ( F T V - I TV ) / ITV ] \times 100$$

Tumour Growth (%) was calculated for each animal in a study group and the group response was calculated as the median response of all treated mice.

For each treatment group a determination was made whether there was a response or no response to the test agent. Response criteria are based on RECIST 1.1 where a response is based on a 30% decrease in tumour volume from baseline tumour measurements.

### Results

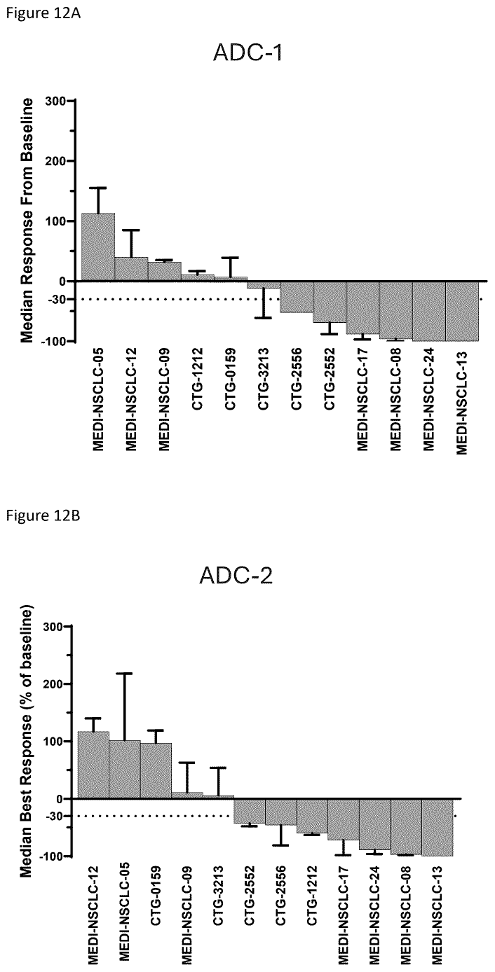

As shown in FIGS. 11A-B, 17 PDX models responded to ADC-1 and 14 PDX models responded to ADC-2, providing overall response rates of 77% and 64% respectively. FIGS. 11C-D show response rates of 74% and 66% for ADC-1 and ADC-2 respectively in further experiments utilizing additional PDX models.

### Example 12: Antitumor Efficacy of ADC-1 and ADC-2 in Patient-Derived Xenograft Models of Squamous Non-Small Cell Lung Cancer

12 sqNSCLC PDX models were initially selected for investigation; 24 sqNSCLC PDX models were investigated in total. Athymic nude or CB17 SCID mice were implanted subcutaneously with tumour tissue fragments. When tumors reached 100-300 mm³, animals were randomised by tumour volume and received a single IV dose of ADC-1 at 2 mg/kg or ADC-2 at 4 mg/kg in order to account for the DAR difference. Tumor volume was measured twice weekly with calipers during the length of the study. Tumor response was measured as a percentage of tumor volume from baseline. The response at the final tumour volume (FTV) from initial tumour volume (ITV) was calculated at the time that resulted in the greatest decrease from the initial tumour volume. The antitumour response was calculated for each tumour bearing mouse using the formula:

$$Tumour Growth ( \% ) = [ ( F T V - I TV ) / ITV ] \times 100$$

Tumour Growth (%) was calculated for each animal in a study group and the group response was calculated as the median response of all treated mice.

For each treatment group a determination was made whether there was a response or no response to the test agent. Response criteria are based on RECIST 1.1 where a response is based on a 30% decrease in tumour volume from baseline tumour measurements.

### Results

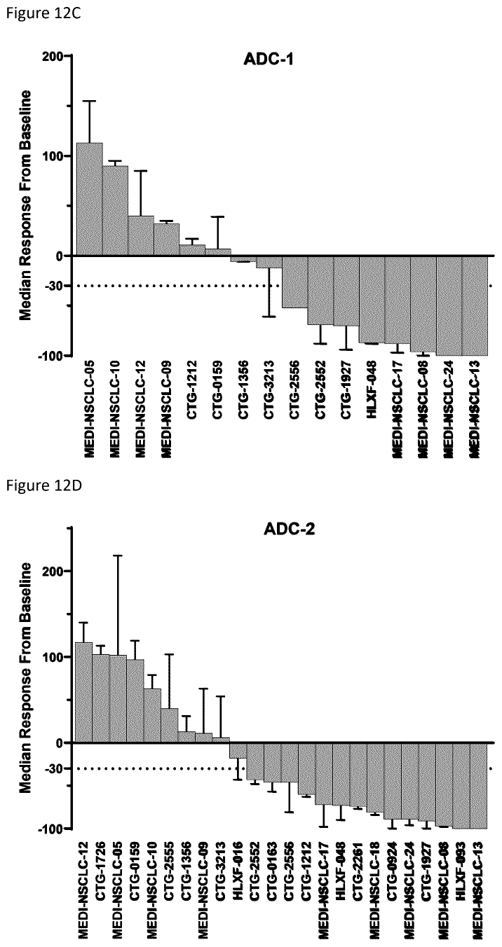

As shown in FIGS. 12A-B, 6 PDX models responded to ADC-1 and 7 PDX models responded to ADC-2, providing overall response rates of 50% and 58% respectively. Identical response rates were observed in experiments utilizing additional PDX models (FIGS. 12C-D).

### Example 13: Antitumor Efficacy of ADC-1 and ADC-2 in Patient-Derived Xenograft Models of Ovarian Cancer

10 ovarian cancer PDX models were selected for investigation. NSG mice were implanted subcutaneously with tumour tissue fragments. When tumors reached 100-300 mm³, animals were randomised by tumour volume and received a single IV dose of ADC-1 at 2 mg/kg or ADC-2 at 4 mg/kg in order to account for the DAR difference. Tumor volume was measured twice weekly with calipers during the length of the study. Tumor response was measured as a percentage of tumor volume from baseline. The response at the final tumour volume (FTV) from initial tumour volume (ITV) was calculated at the time that resulted in the greatest decrease from the initial tumour volume. The antitumour response was calculated for each tumour bearing mouse using the formula:

$$Tumour Growth ( \% ) = [ ( F T V - I TV ) / ITV ] \times 100$$

Tumour Growth (%) was calculated for each animal in a study group and the group response was calculated as the median response of all treated mice.

For each treatment group a determination was made whether there was a response or no response to the test agent. Response criteria are based on RECIST 1.1 where a response is based on a 30% decrease in tumour volume from baseline tumour measurements.

### Results

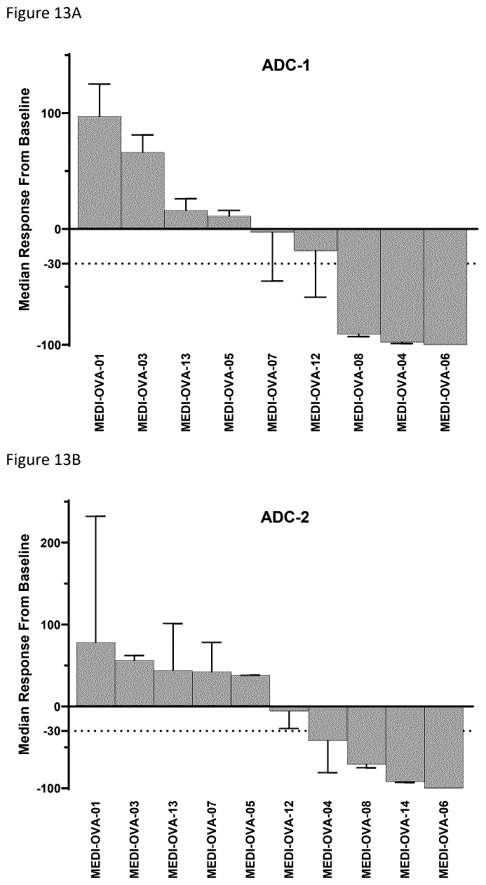

As shown in FIGS. 13A-B, 3 of 9 PDX models responded to ADC-1 and 4 of 10 PDX models responded to ADC-2, providing overall response rates of 33% and 40% respectively.

### Example 14: Antitumor Efficacy of ADC-1 and ADC-2 in Patient-Derived Xenograft Models of Endometrial Cancer

15 endometrial cancer PDX models were selected for investigation. Athymic nude or CB17 SCID mice were implanted subcutaneously with tumour tissue fragments. When tumors reached 100-300 mm³, animals were randomised by tumour volume and received a single IV dose of ADC-1 at 2 mg/kg or ADC-2 at 4 mg/kg in order to account for the DAR difference. Tumor volume was measured twice weekly with calipers during the length of the study. Tumor response was measured as a percentage of tumor volume from baseline. The response at the final tumour volume (FTV) from initial tumour volume (ITV) was calculated at the time that resulted in the greatest decrease from the initial tumour volume. The antitumour response was calculated for each tumour bearing mouse using the formula:

$$Tumour Growth ( \% ) = [ ( F T V - I TV ) / ITV ] \times 100$$

Tumour Growth (%) was calculated for each animal in a study group and the group response was calculated as the median response of all treated mice.

For each treatment group a determination was made whether there was a response or no response to the test agent. Response criteria are based on RECIST 1.1 where a response is based on a 30% decrease in tumour volume from baseline tumour measurements.

### Results

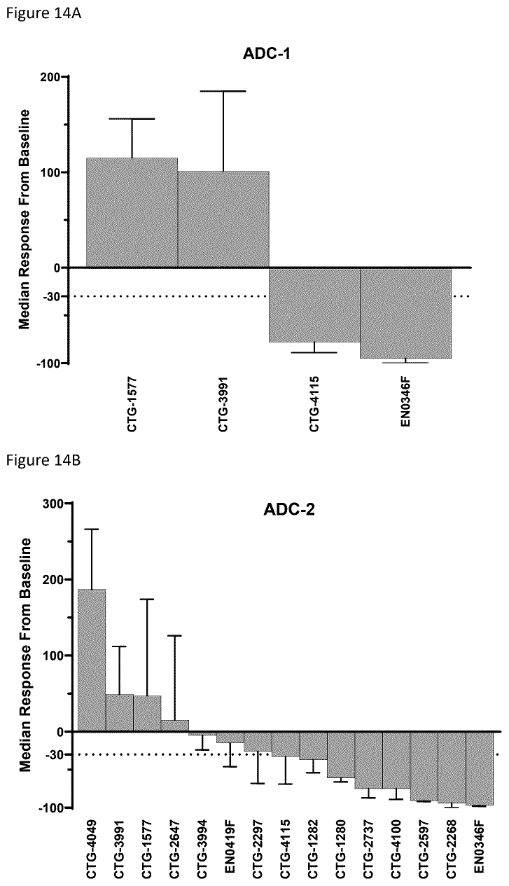

As shown in FIGS. 14A-B, 2 of 4 PDX models responded to ADC-1 and 8 of 15 PDX models responded to ADC-2, providing overall response rates of 50% and 53% respectively.

1-11. (canceled)

12. A conjugate of Formula (I) 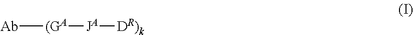
or a pharmaceutically acceptable salt thereof, wherein
Ab is an antibody that binds human B7H3, or an antigen binding fragment thereof that binds human B7H3,
k is an integer from 1 to 10,
each G^{A} is independently a conjugation group conjugated to the antibody or antigen-binding fragment thereof,
each D^{R} is independently a drug comprising a nitrogen atom N^{R},
each J^{A} is independently a group of Formula (IA) 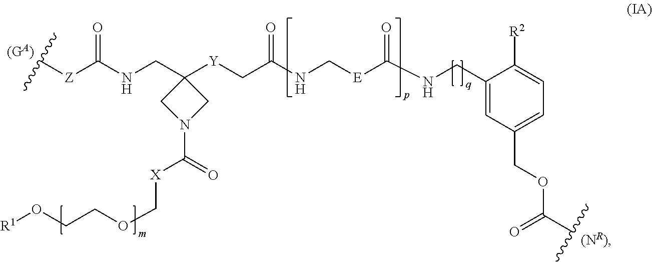
E is (CH₂)ₙ₁, wherein n1 is 0, 1, 2 or 3,
R¹ is C₁₋₄ alkyl or H,
R² is 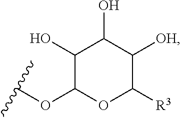
wherein R³ is CO₂H or CH₂OH,
X is (CH₂)ₙ₂, 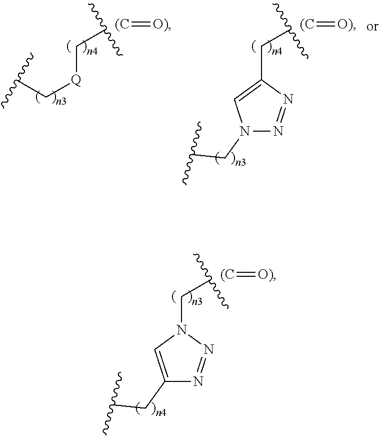
wherein n2 and n4 are 0, 1, 2 or 3, n3 is 1, 2 or 3, and Q is O or NR^{A}, wherein R^{A} is H, C₁₋₄ alkyl or C₃₋₄ cycloalkyl,
Y is O or NR^{B}, wherein R^{B} is H, C₁₋₄ alkyl or C₃₋₄ cycloalkyl,
Z is (CH₂)ₙ₅, wherein n5 is 0, 1, 2, 3, 4 or 5,
m is an integer from 2 to 17,
p is 1 or 0,
q is 1 or 0,
(G^{A}) indicates the point of attachment to G^{A}, and
(N^{R}) indicates the point of attachment to the nitrogen atom N^{R}.

13. The conjugate or a pharmaceutically acceptable salt thereof according to claim 12, wherein the antibody or antigen-binding fragment thereof comprises (a) a heavy chain variable region (VH) comprising an HCDR1 of SEQ ID NO:1, an HCDR2 of SEQ ID NO:2 and an HCDR3 of SEQ ID NO:3, and (b) a light chain variable region (VL) comprising a LCDR1 of SEQ ID NO:4, a LCDR2 of SEQ ID NO:5 and a LCDR3 of SEQ ID NO:6.

14-43. (canceled)

44. The conjugate or a pharmaceutically acceptable salt thereof according to claim 13, wherein
the VH comprises an amino acid sequence having at least 80%, at least 85%, at least 90%, at least 95%, at least 98%, at least 99%, or 100% sequence identity to the amino acid sequence of SEQ ID NO: 7; or
the VL comprises an amino acid sequence having at least 80%, at least 85%, at least 90%, at least 95%, at least 98%, at least 99%, or 100% sequence identity to the amino acid sequence of SEQ ID NO:8.

45. The conjugate or a pharmaceutically acceptable salt thereof according to claim 44, wherein the VH comprises an amino acid sequence of SEQ ID NO: 7 and the VL comprises an amino acid sequence of SEQ ID NO: 8.

46. The conjugate or a pharmaceutically acceptable salt thereof according to claim 13, wherein the antibody or antigen binding fragment thereof comprises:
a heavy chain sequence comprising an amino acid sequence having at least 80%, at least 85%, at least 90%, at least 95%, at least 98%, at least 99%, or 100% sequence identity to the amino acid sequence of SEQ ID NO: 9 and/or a light chain sequence comprising an amino acid sequence having at least 80%, at least 85%, at least 90%, at least 95%, at least 98%, at least 99%, or 100% sequence identity to the amino acid sequence of SEQ ID NO: 11; or
a heavy chain sequence comprising an amino acid sequence having at least 80%, at least 85%, at least 90%, at least 95%, at least 98%, at least 99%, or 100% sequence identity to the amino acid sequence of SEQ ID NO: 10 and/or a light chain sequence comprising an amino acid sequence having at least 80%, at least 85%, at least 90%, at least 95%, at least 98%, at least 99%, or 100% sequence identity to the amino acid sequence of SEQ ID NO: 11.

47. The conjugate or a pharmaceutically acceptable salt thereof according to claim 46, wherein the antibody comprises a heavy chain sequence of SEQ ID NO: 9 and a light chain sequence of SEQ ID NO: 11.

48. The conjugate or a pharmaceutically acceptable salt thereof according to claim 46, wherein the antibody comprises a heavy chain sequence of SEQ ID NO: 10 and a light chain sequence of SEQ ID NO:11.

49. The conjugate or pharmaceutically acceptable salt thereof according to claim 12, wherein m is 9, 10, 11, 12 or 13.

50. The conjugate or pharmaceutically acceptable salt thereof according to claim 12, wherein R¹ is CH₃.

51. The conjugate or pharmaceutically acceptable salt thereof according to claim 12, wherein R² is

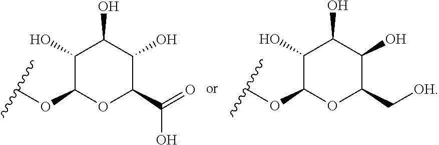 [SMILES: *O[C@@H]1O[C@H](C(=O)O)[C@@H](O)[C@H](O)[C@H]1O ; "or" ; *O[C@@H]1O[C@H](CO)[C@H](O)[C@H](O)[C@H]1O]

52. The conjugate or pharmaceutically acceptable salt thereof according to claim 12, wherein
E is CH₂;
X is CH₂;
Y is O;
Z is (CH₂)₂;
p is 1; and/or
q is 1.

53. The conjugate or pharmaceutically acceptable salt thereof according to claim 12, wherein J^{A} is a group of Formula (IB)

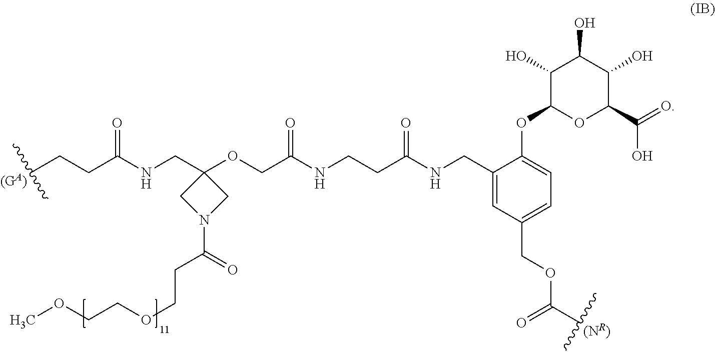

54. The conjugate or a pharmaceutically acceptable salt thereof according to claim 12, wherein G^{A} is selected from 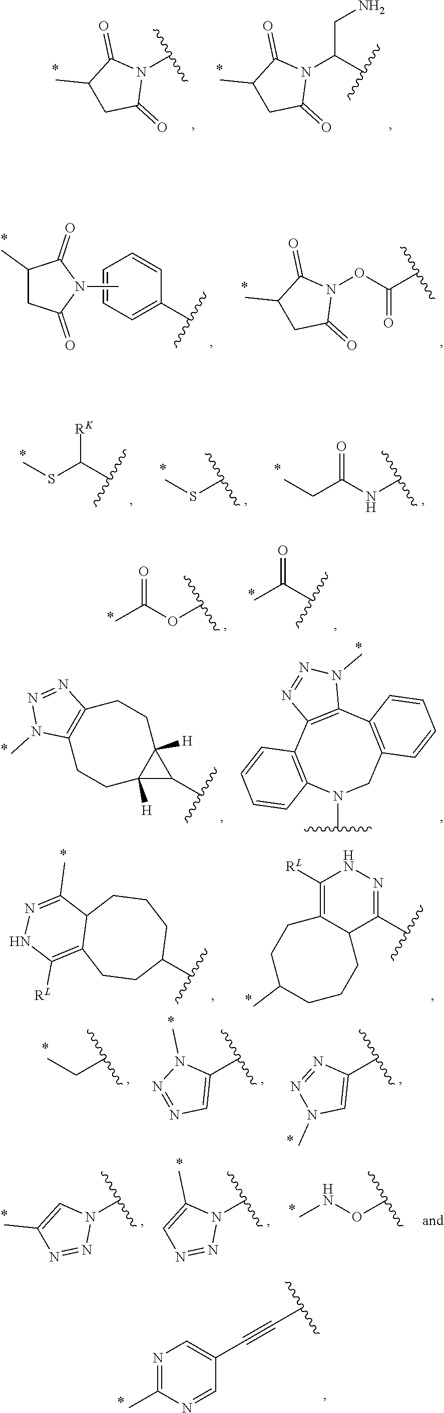
wherein R^{K} is H or CH₃, R^{L} is C₁₋₆ alkyl, and 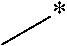 [SMILES: *C]
indicates the point of attachment to the antibody or antigen-binding fragment thereof.

55. The conjugate or pharmaceutically acceptable salt thereof according to claim 12, wherein G^{A} is 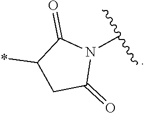 [SMILES: *C1CC(=O)N(*)C1=O]
wherein 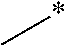 [SMILES: *C]
indicates the point of attachment to the antibody or antigen-binding fragment thereof.

56. The conjugate or pharmaceutically acceptable salt according to claim 12, wherein D^{R} is

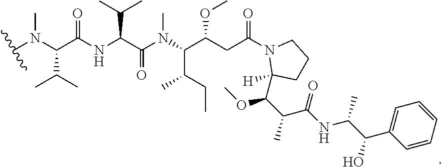 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

57. The conjugate or pharmaceutically acceptable salt thereof according to claim 12, wherein each G^{A}-J^{A}-D^{R} is a group of Formula (IC) 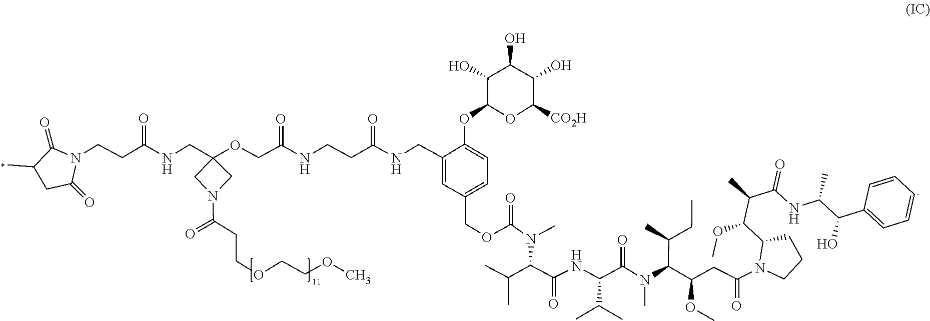
wherein 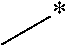 [SMILES: *C]
indicates the point of attachment to the antibody or antigen-binding fragment thereof.

58. The conjugate or pharmaceutically acceptable salt thereof according to claim 12, wherein k is an integer from 2 to 6.

59. The conjugate or pharmaceutically acceptable salt thereof according to claim 12, wherein k is 2.

60. The conjugate or pharmaceutically acceptable salt thereof according to claim 12, wherein k is 4.

61. A conjugate of Formula (I) 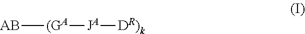
or a pharmaceutically acceptable salt thereof, wherein
Ab is an antibody that binds human B7H3 comprising a heavy chain sequence of SEQ ID NO: 9 and a light chain sequence of SEQ ID NO: 11,
k is 2, and
each G^{A}-J^{A}-D^{R} is a group of Formula (IC) 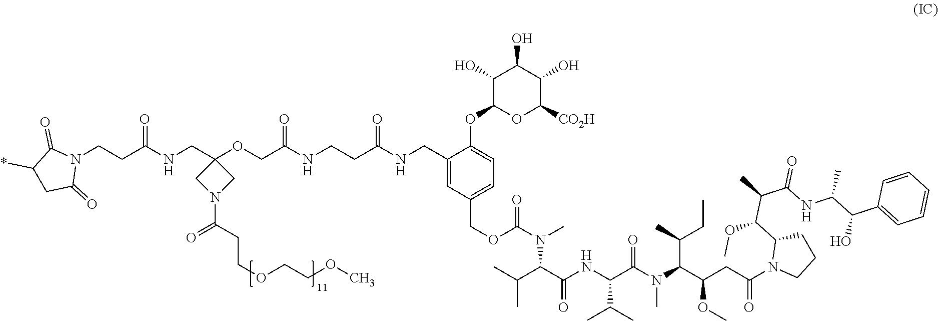
wherein 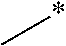 [SMILES: *C]
indicates the point of attachment to the antibody.

62. The conjugate of claim 61, wherein each G^{A}-J^{A}-D^{R} group is conjugated to a cysteine at position 174 of SEQ ID NO: 9.

63. A conjugate of Formula (I) 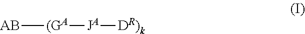
or a pharmaceutically acceptable salt thereof, wherein
Ab is an antibody that binds human B7H3 comprising a heavy chain sequence of SEQ ID NO: 10 and a light chain sequence of SEQ ID NO: 11,
k is 4, and
each G^{A}-J^{A}-D^{R} is a group of Formula (IC) 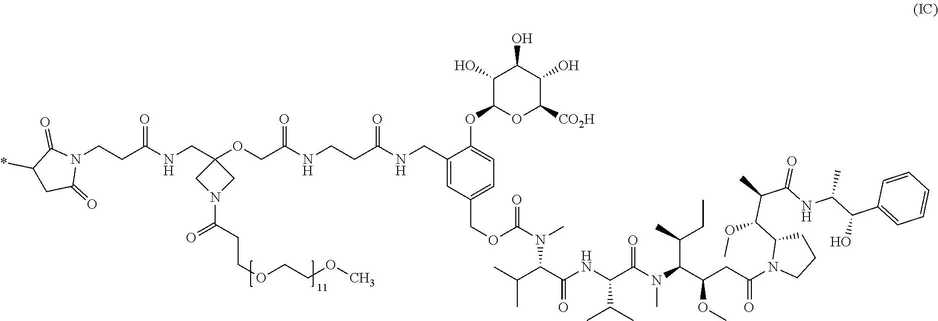
wherein 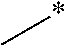 [SMILES: *C]
indicates the point of attachment to the antibody.

64. A pharmaceutical composition comprising the conjugate or a pharmaceutically acceptable salt thereof according to claim 12.

65. A method of treating a disease or disorder in a patient in need thereof comprising administering to the patient a therapeutically effective amount of the conjugate or pharmaceutically acceptable salt thereof according to claim 12.

66. The method according to claim 65, wherein the disease or disorder is cancer.

67. The method according to claim 66, wherein the cancer is a B7H3-expressing cancer.

68. The method according to claim 67, wherein the cancer is a solid tumour, such as lung cancer (such as non-small cell lung cancer (NSCLC), squamous cell non-small cell lung cancer (sqNSCLC), adenocarcinoma non-small cell lung cancer (adNSCLC) or small cell lung cancer (SCLC)), prostate cancer, metastatic castration-resistant prostate cancer (mCRPC), metastatic hormone-sensitive prostate cancer (mHSPC), neuroendocrine prostate cancer (NEPC), esophageal cancer, esophageal squamous-cell carcinoma (ESCC), esophageal adenocarcinoma, bladder cancer, endometrial cancer, skin cancer, melanoma, cutaneous squamous cell carcinoma, sarcoma, head and neck cancer, head and neck squamous cell carcinoma (HNSCC), salivary gland cancer, oral cancer, oral squamous cell carcinoma, breast cancer, liver cancer, hepatocellular carcinoma (HCC), intrahepatic cholangiocarcinoma, ovarian cancer, colorectal cancer, pancreatic cancer, kidney cancer, renal cell carcinoma (RCC), biliary tract cancer, cholangiocarcinoma, intrahepatic cholangiocarcinoma, gastric cancer, thyroid cancer, cervical cancer, brain cancer, glioma, adrenal cancer, adrenocortical carcinoma.


SEQUENCE LISTING (14 sequences)

SEQ ID NO: 1 - protein, 5 aa, synthetic construct
  source 1..5: note = HCDR1
SYAMS

SEQ ID NO: 2 - protein, 17 aa, synthetic construct
  source 1..17: note = HCDR2
AISGSGGSTYYADSVKG

SEQ ID NO: 3 - protein, 12 aa, synthetic construct
  source 1..12: note = HCDR3
DLGSGRGRHFDS

SEQ ID NO: 4 - protein, 14 aa, synthetic construct
  source 1..14: note = LCDR1
TGSSSNIGAGYDVH

SEQ ID NO: 5 - protein, 7 aa, synthetic construct
  source 1..7: note = LCDR2
GNSNRPS

SEQ ID NO: 6 - protein, 11 aa, synthetic construct
  source 1..11: note = LCDR3
QSFDSSELWST

SEQ ID NO: 7 - protein, 121 aa, synthetic construct
  source 1..121: note = VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLGSGRGRHFDSWGQGTLVTVSS

SEQ ID NO: 8 - protein, 111 aa, synthetic construct
  source 1..111: note = VL
QAVLTQPPSVSGAPGQRVTISCTGSSSNIGAGYDVHWYQQLPGTAPKLLIYGNSNRPSGVPDRFSGSKSGTSASLAITGLQAEDEADYYCQSFDSSELWSTFGGGTKVTVL

SEQ ID NO: 9 - protein, 451 aa, synthetic construct
  source 1..451: note = HC(L174C)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLGSGRGRHFDSWGQGTLVTVSSASTKGPSVFPLAPSSKSTSGGTAALGCLVKDYFPEPVTVSWNSGALTSGVHTFPAVCQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 451 aa, synthetic construct
  source 1..451: note = HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLGSGRGRHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 217 aa, synthetic construct
  source 1..217: note = LC
QAVLTQPPSVSGAPGQRVTISCTGSSSNIGAGYDVHWYQQLPGTAPKLLIYGNSNRPSGVPDRFSGSKSGTSASLAITGLQAEDEADYYCQSFDSSELWSTFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - DNA, 651 bp, synthetic construct
  source 1..651: mol_type = other DNA; note = LC
caagctgttctgacacagcctcctagtgtgtctggcgctcctggccagagagtgaccatctcttgtaccggcagcagcagcaatatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgccagagcttcgacagcagcgagctgtggtctacatttggcggaggcaccaaagtgaccgtgctgggacaacctaaggcggcgcctagcgtgaccctgtttcctccaagcagcgaggaactgcaggctaacaaggccacactcgtgtgcctgatcagcgacttctatcccggcgctgtgaccgtggcctggaaagctgatagctctcctgtgaaggccggcgtggaaaccaccacacctagcaagcagagcaacaacaaatacgccgccagcagctacctgagcctgacacctgagcagtggaagtcccacagatcctacagctgccaagtgacccacgagggcagcaccgtggaaaagacagtggcccctacagagtgttca

SEQ ID NO: 13 - DNA, 1353 bp, synthetic construct
  source 1..1353: mol_type = other DNA; note = HC(L174C)
gaggtgcagctgcttgaatctggcggaggactggttcagcctggcggatctctgagactgtcttgtgccgccagcggcttcacctttagcagctacgccatgagctgggtccgacaggctcctggcaaaggccttgaatgggtgtccgccatctctggcagcggcggcagcacatattacgccgattctgtgaagggcagattcaccatcagccgggacaacagcaagaacaccctgtacctccagatgaacagcctgagagccgaggacaccgccgtgtactattgtgcccgcgatctcggctctggcagaggcagacactttgattcttggggccagggcaccctggtcaccgttagttctgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtctgtcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa

SEQ ID NO: 14 - DNA, 1353 bp, synthetic construct
  source 1..1353: mol_type = other DNA; note = HC
gaggtgcagctgcttgaatctggcggaggactggttcagcctggcggatctctgagactgtcttgtgccgccagcggcttcacctttagcagctacgccatgagctgggtccgacaggctcctggcaaaggccttgaatgggtgtccgccatctctggcagcggcggcagcacatattacgccgattctgtgaagggcagattcaccatcagccgggacaacagcaagaacaccctgtacctccagatgaacagcctgagagccgaggacaccgccgtgtactattgtgcccgcgatctcggctctggcagaggcagacactttgattcttggggccagggcaccctggtcaccgttagttctgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaattcgaggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcttaagcctgtctccgggtaaa